(19) United States
(12) Patent Application Publication
(10) Pub. No.: US 2024/0091171 A1
(43) Pub. Date: Mar. 21, 2024

(54) DERMATOLOGICAL COMPOSITIONS AND USES THEREOF

(71) Applicant: Reza Babapour, Beverly Hills, CA (US)

(72) Inventor: Reza Babapour, Beverly Hills, CA (US)

(21) Appl. No.: 18/301,156

(22) Filed: Apr. 14, 2023

Related U.S. Application Data
(63) Continuation of application No. PCT/US2021/071894, filed on Oct. 14, 2021.
(60) Provisional application No. 63/091,810, filed on Oct. 14, 2020.

(51) Int. Cl. A61K 31/07; A61K 9/00; A61K 31/366; A61K 31/404

(52) U.S. Cl. CPC A61K 31/07; A61K 9/0014; A61K 31/366; A61K 31/404

(57) ABSTRACT

Methods for treating or preventing microbial infection, methods for treating or preventing a dermatological condition caused by environmental or pathological factors, and compositions for use in such methods are disclosed.

### CROSS-REFERENCE TO RELATED APPLICATIONS

This application is a continuation of International Application No. PCT/US2021/071894, filed Oct. 14, 2021, which claims the benefit of U.S. Provisional Patent Application No. 63/091,810, filed Oct. 14, 2020, both of which are incorporated herein by reference in its entirety.

### BACKGROUND

There are many dermatological products marketed and prescribed to prevent microbial infection and to protect and repair damaged, sensitive, and aging skin. However, many such products are limited in efficacy due to side effects or inability to protect the skin's ability to maintain its barrier function. Other products are undesirable to many users because the product contains harsh or unnatural products. Thus, there is an interest in developing dermatological products that provide a natural and effective way to treat and prevent dermatological conditions and infection.

### DETAILED DESCRIPTION

Described herein are methods for treating or preventing microbial infection, methods for treating or preventing a dermatological condition caused by environmental or pathological factors, and compositions for use in such methods.

Dermatological Compositions and Components Thereof

According to the embodiments described herein, dermatological conditions and microbial infections can be treated or prevented using a dermatological composition such as a personal care composition, a skin care composition, an oral care composition, a cosmetic composition, or a pharmaceutical composition. Such compositions are not mutually exclusive. In other words, one type of composition described herein may serve as any one or more of the other types of compositions. Specific examples of compositions that may be used in the embodiments described herein are discussed further below.

In some embodiments, the composition includes, but is not limited to, one or more primary agents selected from the following agents or groups of agents: retinoids, astaxanthin, arvelexin, plant-derived biopolymers and extracts thereof (e.g., cutin, lignin, suberin), lysozyme, and nisin. In certain such embodiments, the one or more primary agents include a first primary agent and, optionally, one or more supplemental agents.

In some embodiments, the composition includes two or more primary agents selected from the following agents or groups of agents: retinoids, astaxanthin, arvelexin, plant-derived biopolymers and extracts thereof (e.g., cutin, suberin, lignin), lysozyme, and nisin. In certain such embodiments, the two or more primary agents include a first primary agent, a second primary agent and, optionally, one or more supplemental agents.

In some embodiments, the composition includes three or more primary agents selected from the following agents or groups of agents: retinoids, astaxanthin, arvelexin, plant-derived biopolymers and extracts thereof (e.g., cutin, suberin, lignin), lysozyme, and nisin. In certain such embodiments, the three or more primary agents include a first primary agent, a second primary agent, a third primary agent, and, optionally, one or more supplemental agents.

In some embodiments, the first and/or second and/or third primary agent of the composition is a retinoid, which are synthetic and natural biologically active forms of vitamin A and synthetic analogues or vitamin A. Retinoids are a class of compounds useful for treating disorders of the skin, including acne (e.g., acne vulgaris, cystic acne, acne conglobata, and acne rosacea), warts, condyloma, flat warts (verruca plana), plantar warts, verruca vulgaris, flat warts, verruca plana, molluscum contagiosum, dyshidrotic eczema (e.g., eczema of the hands and feet), atopic dermatitis, atopic dermatitis, psoriasis, seborrheic dermatitis, aged skin (e.g., photoaged skin or chronologically aged skin), skin cancer, hyperpigmentation, melasma, cutaneous lesions of AIDS-related Kaposi's sarcoma, keratinization disorders (e.g., ichthyosis, Pityriasis rubra pilaris, and Darier's disease, Grover's disease, plantar hyperkeratosis (and other hyperkeratosis conditions), pitted keratolysis, seborrheic keratosis, keratosis pilaris), acanthosis nigricans, carcinoma (e.g., basal cell and squamous cell carcinomas, cutaneous T-cell lymphoma (mycosis fungoides), precancerous actinic keratosis, porokeratosis, bowenoid papulosis, keratoacanthoma, and cutaneous sarcoidosis, hidradenitis suppurativa. Retinoids are also useful for treating and/or preventing dermatological disorders such as wrinkles, age related skin changes, abnormal or diminished synthesis of collagen and elastin, diminished levels of collagen and elastin in the dermis, stretch marks, improvement of skin lines, fine lines, fine and coarse wrinkles, tactile roughness, thinning of skin (skin atrophy), skin thickening due to elastosis of photoaging, loss or reduction of skin resiliency, elasticity and recoilability, lack of skin resiliency, and older looking skin, nodular elastoidosis. Retinoids also are used to treat pigmentary skin changes such as freckles, ephelides, solar lentigo, and melasma. Retinoids work by encouraging basal cells to divide, resulting in new epidermal cells that migrate to the skin's surface, then causing the exfoliation process to shed the excess skin. But, with overuse, new skin cells do not function properly because they are produced rapidly lack the necessary adhesion and lipid production to protect the skin properly. Thus, the more retinoid that is used, the less effective the barrier function of the top layer of skin becomes. Users tend to experience increased skin sensitivity (e.g., main effect is an increase in sensitivity to UV sunlight, (i.e., photosensitization), peeling, flaking, irritation. Additionally, administration of retinoids—even from the first dose—is associated with undesirable side effects such as skin irritation, dryness, redness, and epidermal hyperplasia. For example, in keratinocytes, retinoids have been known to induce proliferation, causing epidermal hyperplasia, edema, blistering, crusting, severe local erythema, burning, stinging, pruritus, heightened burning susceptibility upon exposure to sunlight, skin peeling, and overall physical discomfort. These side effects limit the initial and long-term tolerable dosages in users and discourage users from continuing treatment. Many of these side effects associated with loss of barrier function caused by the use of retinoids are associated with an increase in trans-epithelial water loss (TEWL), which is a way to measure the skin's barrier function and response to irritants. According to some embodiments described herein, compositions containing a retinoid as a first primary agent may be combined with one or more additional primary agents (e.g., one or more plant derived biopolymers, discussed below) as second and/or third primary agents to reduce TEWL, thereby improving the skin's barrier function during retinoid treatment and improving a user's tolerance to the retinoid. This improved tolerance has several advantages, including improved user or patient compliance (i.e., the user will continue to use the product without interruption due to side effects), the ability to increase the initial and subsequent dosage of the retinoid as compared to typical standard doses used in products in the field to increase efficacy of the product, and reduced side effects caused by the retinoid. Additionally, the use of higher initial and subsequent retinoid concentrations allows for the user to achieve a quicker onset of a desirable therapeutic response.

Several types of retinoids may be used as the first and/or second and/or third primary agent in the compositions described herein. In some embodiments, the retinoid may be any suitable therapeutically effective retinoid or derivative thereof including, but not limited to, retinyl palmitate, retinol, retinaldehyde (also known as retinal or vitamin A aldehyde), tretinoin (also known as retinoic acid or vitamin A acid), acitretin, adapalene, alitretinoin, bexarotene, etretinate, isotretinoin, trifarotene, and tazarotene.

Retinoids can be administered both topically and systemically. In some embodiments, the concentration or dose of the retinoid used in the compositions described herein is selected at least in part based on the type of retinoid the application for which it is used, and the route of administration. For example, in certain embodiments, a composition that includes a retinoid for topical administration may be formulated to include less than 0.001%, between about 0.001% and 0.10%, less than 0.10% (e.g., about 0.01%, about 0.025%, about 0.05%, about 0.075%, about 0.08%-0.085%, about 0.085-0.09%, 0.095%), about 0.10%, between about 0.10% and 1% (e.g., about 0.10-0.15%, about 0.15-0.20%, about 0.20-0.25%, about 0.25-0.30%, about 0.30-0.35%, about 0.35-0.40%, about 0.40-0.45%, about 0.45-0.50%, about 0.50-0.55%, about 0.55-0.60%, about 0.60-0.65%, about 0.65-0.70%, about 0.70-0.75%, about 0.75-0.80%, about 0.80-0.85%, about 0.85-0.90%, about 0.90-0.95%, about 0.95-1%), about 1.0%, between about 1.0-2%, about 2%, between about 2.0-3%, about 3%, between about 3%-4%, about 4%, between about 4%-5%, about 5%, between about 5%-6%, about 6%, between about 6%-7%, about 7%, or more than about 7% of a retinoid, by weight or volume of the composition. The exact content (%) of retinoid to be used in the compositions will depend on the particular retinoid utilized since such agents vary in potency and are tolerated at varying doses when used alone or in combination with additional agents described herein.

In some embodiments, the retinoid used as the first and/or second and/or third primary agent is retinol. The retinol may be included in the composition at any suitable concentration of retinoids disclosed herein. In certain embodiments in which retinol is used, the composition includes from about 0.001% to about 0.05%, from about 0.001% to about 0.1%, from about 0.001% to about 1%, from 0.001% to about 1.5%, from 0.001% to about 2%, from 0.001% to about 3%, from 0.001% to about 4%, from 0.001% to about 5%, from 0.001% to about 6%, or more than 6% or about 7% or more of retinol, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 6%, or about 7%, or more of retinol, by weight or volume of the composition.

In some embodiments, the retinoid used as the first and/or second and/or third primary agent is a retinyl ester (e.g., retinyl palmitate, retinyl acetate). The retinyl ester may be included in the composition at any suitable concentration of retinoids disclosed herein. In certain embodiments in which a retinyl ester is used, the composition includes from about 0.001% to about 0.05%, from about 0.001% to about 0.1%, from about 0.001% to about 1%, more than 1%, more than 2%, more than 3%, more than 4%, more than 5%, more than 6%, or about 7% or more of retinyl ester, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 6%, or about 7% or more, of retinyl ester, by weight or volume of the composition.

In some embodiments, the retinoid used as the first and/or second and/or third primary agent is retinaldehyde. The retinaldehyde may be included in the composition at any suitable concentration of retinoids disclosed herein. In certain embodiments in which retinaldehyde is used, the composition includes from about 0.001% to about 0.05%, from about 0.001% to about 0.1%, from about 0.001% to about 1%, or more than 1%, of retinaldehyde, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 6%, or about 7% or more of retinaldehyde, by weight or volume of the composition.

In some embodiments, the retinoid used as the first and/or second and/or third primary agent is tretinoin. The tretinoin may be included in the composition at any suitable concentration of retinoids disclosed herein. In certain embodiments in which tretinoin is used, the composition includes from about 0.001% to about 0.05%, from about 0.001% to about 0.1%, from about 0.001% to about 1%, or more than 1%, of tretinoin, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.075%, about 0.08%-0.085%, about 0.085-0.09%, 0.095%), about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 6%, or about 7% or more of tretinoin, by weight or volume of the composition.

In some embodiments, the retinoid used as the first and/or second and/or third primary agent is adapalene. The adapalene may be included in the composition at any suitable concentration of retinoids disclosed herein. In certain embodiments in which adapalene is used, the composition includes from about 0.001% to about 0.05%, from about 0.001% to about 0.1%, from about 0.001% to about 1%, or more than 1%, of adapalene, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, or about 5%, of adapalene by weight or volume of the composition.

In some embodiments, the retinoid used as the first and/or second and/or third primary agent is trifarotene. The trifarotene may be included in the composition at any suitable concentration of retinoids disclosed herein. In certain embodiments in which trifarotene is used, the composition includes from about 0.001% to about 0.05%, from about 0.001% to about 0.1%, from about 0.001% to about 1%, or more than 1%, of trifarotene, by weight or volume of the composition. In certain embodiments, the composition includes about 0.001%, about 0.005%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, or about 5% of trifarotene, by weight or volume of the composition.

In some embodiments, the retinoid used as the first and/or second and/or third primary agent is tazarotene. The tazarotene may be included in the composition at any suitable concentration of retinoids disclosed herein. In certain embodiments in which tazarotene is used, the composition includes from about 0.001% to about 0.05%, from about 0.001% to about 0.1%, from about 0.001% to about 1%, or more than 1%, of tazarotene, by weight or volume of the composition. In certain embodiments, the composition includes about 0.001%, about 0.005%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, or about 5% of tazarotene, by weight or volume of the composition.

In some embodiments, the retinoid used as the first and/or second and/or third primary agent is alitretinoin. The alitretinoin may be included in the composition at any suitable concentration of retinoids disclosed herein. In certain embodiments in which alitretinoin is used, the composition includes from about 0.001% to about 0.05%, from about 0.001% to about 0.1%, from about 0.001% to about 1%, or more than 1%, of alitretinoin, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, or about 5% of alitretinoin, by weight or volume of the composition. In certain embodiments, alitretinoin is administered orally, in which embodiments the composition includes about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg, of alitretinoin.

In some embodiments, the retinoid used as the first and/or second and/or third primary agent is bexarotene. The bexarotene may be included in the composition at any suitable concentration of retinoids disclosed herein. In certain embodiments in which bexarotene is used, the composition includes from about 0.001% to about 0.05%, from about 0.001% to about 0.1%, from about 0.001% to about 1%, or more than 1%, of bexarotene, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, or about 5% or more of bexarotene, by weight or volume of the composition. In certain embodiments, bexarotene is administered orally, in which embodiments the composition includes about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, of bexarotene.

In some embodiments, the retinoid used as the first and/or second and/or third primary agent is isotretinoin. The isotretinoin may be included in the composition at any suitable concentration of retinoids disclosed herein. In certain embodiments in which isotretinoin is used, the composition includes from about 0.001% to about 0.05%, from about 0.001% to about 0.1%, from about 0.001% to about 1%, or more than 1%, of isotretinoin, by weight or volume of the composition. In certain embodiments, the composition includes about 0.001%, about 0.01%, about 0.015%, about 0.02%, about 0.025%, about 0.03%, about 0.035%, about 0.04%, about 0.045%, about 0.05%, about 0.1%, about 0.15%, about 0.2%, about 0.25%, about 0.3%, about 0.35%, about 0.4%, about 0.45%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 6%, or about 7% or more of isotretinoin, by weight or volume of the composition. In certain embodiments, isotretinoin is administered orally, in which embodiments the composition includes about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg, of isotretinoin.

In some embodiments, the retinoid used as the first and/or second and/or third primary agent is acitretin. The acitretin may be included in the composition at any suitable concentration of retinoids disclosed herein. In certain embodiments, acitretin is administered orally, in which embodiments the composition includes about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 17.5 mg, about 18 mg, about 19 mg, about 20 mg, about 21 mg, about 22 mg, about 22.5 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg, of acitretin.

In some embodiments, the first and/or second and/or third primary agent of the composition is astaxanthin or a derivative thereof. Astaxanthin is a ketocarotenoid having a red-orange pigment that was originally isolated from a lobster, but is also found in shrimp, salmon, and crayfish. Astaxanthin has several protective properties, including antioxidant properties, anti-inflammatory properties, immune-enhancing properties, antimutagenic, anti-cancer/anti-neoplastic/anti-tumor, and properties related to the prevention of skin damage—including prevention and repair of damage due to UV radiation (see, e.g., Davinelli et al. 2018). Astaxanthin also has hypolipidemic properties, provides protection against bone resorption and loss of bone density, and is known to enhance wound healing and to promote repair of damaged tissues and wounds (see, e.g., Kishimoto et al. 2016; Hwang et al. 2018; Meephansan et al. 2017).

Astaxanthin is naturally synthesized by a number of plants, bacteria, microalgae, phytoplankton, and yeasts, but the microalga Haematococcus pluvalis is considered to have the highest capacity to produce astaxanthin and thus provides the main source for human applications. For cost-efficiency in mass production, astaxanthin can also be synthetically produced using petrochemicals. The astaxanthin used in the compositions described herein may be sourced from an organism from which it is naturally synthesized or may be synthetically produced. Astaxanthin exist in several forms, all of which may be used as a first and/or second and/or third primary agent of the composition. For example, in some embodiments, the astaxanthin used in the embodiments described herein is free astaxanthin, chelated astaxanthin, or an astaxanthin ester (e.g., astaxanthin monoester or astaxanthin diester). In another example, in some embodiments, the astaxanthin used in the embodiments described herein is provided as one or the following astaxanthin stereoisomers: 3S, 3′S; 3R, 3′R; or 3R, 3′S, each of which differs from each other by the configuration of the two hydroxyl groups on the molecule. In another example, in some embodiments, astaxanthin is provided as an oil, a powder, a water-soluble powder or liquid, a gel or soft gel, or as a natural microalgal product. All forms of astaxanthin discussed herein are collectively referred to herein as “astaxanthin.”

In some embodiments, the concentration of astaxanthin used in the compositions described herein is selected at least in part based on the form of astaxanthin and the application for which it is used. For example, in certain embodiments, a composition that includes astaxanthin for topical administration may be formulated to include between about 0.1% to 1%, between about 0.1% to 2%, between about 0.1% to 3%, between about 0.1% to 4%, between about 0.1% to 5%, between about 0.1% to 6%, between about 0.1% to 7%, between about 0.1% to 8%, between about 0.1% to 9%, between about 0.1% to 10%, between about 0.1% to 15%, or between about 0.1% to 20%, of astaxanthin, by weight or volume of the composition. In other embodiments, a composition that includes astaxanthin for oral administration may be formulated to include between about 1 mg to about 28 mg astaxanthin, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, or more than 28 mg of astaxanthin, by weight or volume of the composition.

In some embodiments, the first and/or second and/or third primary agent of the composition is arvelexin. Arvelexin is an indole compound that is found in the root of Brassica rapa (turnips) (see, e.g., Paul et al. 2018). The major active constituents of turnip are glucosinolates, isothiocyanates, flavonoids and phenylpropanoids which have diverse bioactivities, including antioxidant, anticancer, antimicrobial, antidiabetic, hepatoprotective, nephroprotective, cardiovascular and hypolipidemic, analgesic and anti-inflammatory effects (Id.).

The arvelexin used in the compositions described herein may be sourced from Brassica rapa, or may be synthetically produced (e.g., 7-hydroxyl-1-methylindole-3-acetonitrile (7-HMIA) is a synthesized analog of arvelexin). In some embodiments, the concentration of arvelexin used in the compositions described herein is selected at least in part based on the form of arvelexin and the application for which it is used. For example, in certain embodiments, a composition that includes arvelexin for topical administration may be formulated to include between about 0.1% to 1%, between about 0.1% to 2%, between about 0.1% to 3%, between about 0.1% to 4%, between about 0.1% to 5%, between about 0.1% to 6%, between about 0.1% to 7%, between about 0.1% to 8%, between about 0.1% to 9%, between about 0.1% to 10%, between about 0.1% to 15%, between about 0.1% to 20%, between about 0.1% to 30%, between about 0.1% to 40%, between about 0.1% to 50%, of arvelexin, between 50% to 95%, between 95% or more, by weight or volume of the composition. In other embodiments, a composition that includes arvelexin for topical administration may be formulated to include less than 10 μM, between about 10 μM to 200 μM (to include between about 10 μM to 25 μM, between about 25 μM to 50 μM, between about 50 μM to 75 μM, between about 75 μM to 100 μM, between about 100 μM to 150 μM, between about 150 μM to 200 μM), or more than 200 μM. In other embodiments, a composition that includes arvelexin for topical administration may be formulated to include about 10 μM, about 25 μM, about 50 μM, about 75 μM, about 100 μM, about 150 μM, or about 200 μM.

In some embodiments, the first and/or second and/or third primary agent of the composition is a plant-derived biopolymer or an extract thereof. In certain embodiments, the plant-derived biopolymer is cutin, suberin, or lignin.

In some embodiments, the plant-derived biopolymer is cutin. Cutin is the major structural component of the plant cuticle, and is a biopolyester primarily composed of interesterified hydroxy, and epoxy-hydroxy fatty acid monomers with a chain length of 16 or/and 18 carbons (C₁₆ and C₁₈ class). Other monomers of cutin may be fatty acids, fatty alcohols, aldehydes, ketones, diacids as well as hydroxycinnamic acids. The cutin used in the embodiments described herein may be derived directly from a cutin-containing portion of plant matter (i.e., extracted from a plant cuticle), and in some embodiments, may be cutin-derived monomers or oligomers from a plant. Alternatively, the cutin used in the embodiments herein may be synthetically produced by any suitable method known in the art.

In some embodiments, the plant-derived biopolymer is suberin. Suberin is a plant-specific cell wall-associated hydrophobic polymer containing a fatty acid-derived domain and an aromatic domain, that is found in or secreted by various tissues of underground plant parts and some aerial organs, for example, suberin is secreted by cork cells for sealing a stem against water loss or by various plant tissues in response to biotic or abiotic stresses. Suberin has functional, structural, and chemical similarities to cutin. Pure suberin can be isolated from the bark of cork oak (Quercus suber L). Thus, in some embodiments, the suberin used in the compositions may be purified from an extract derived from cork, for example, Quercus suber extract. Alternatively, the suberin used in the embodiments herein may be synthetically produced by any suitable method known in the art.

In some embodiments, the plant-derived biopolymer is lignin. Lignin is an abundant natural polymer found in lignocellulosic biomass from a variety of plant species. The structure of lignin varies based on the species, but generally corresponds to a three-dimensional, cross-linked macromolecule with substituted phenols (e.g., coniferyl, sinapyl, and p-coumaryl alcohols). The lignin used in the embodiments described herein may be derived directly from a lignin-containing portion of plant matter (i.e., extracted from jute, hemp, cotton, wood pulp, or plant pulp). In certain embodiments, the type of lignin used includes, but is not limited to, soda lignin. Kraft lignin, hydrolyzed lignin, organosolv lignin, and/or lignosulfonates. Alternatively, the lignin used in the embodiments herein may be synthetically produced by any suitable method known in the art.

In certain embodiments, a composition that includes suberin, cutin, or lignin for topical administration may be formulated to include less than 1% (e.g., about 0.1%, 0.2%, 0.3%, 0.4%, 0.5%, 0.6%, 0.7%, 0.8%, 0.9%), between about 0.1% to 1%, between about 1% to 30%, or more than 30% of suberin, cutin, or lignin, by weight or volume of the composition. In certain embodiments, a composition that includes suberin, cutin, or lignin for topical administration may be formulated to include between about 1% to 5%, 5% to 10%, 10% to 15%, 15% to 20%, 20% to 25%, 25% to 30%, or 30% or more of suberin, cutin, or lignin, by weight or volume of the composition. In certain embodiments, a composition that includes suberin, cutin, or lignin for topical administration may be formulated to include about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of suberin, cutin, or lignin, by weight or volume of the composition. In certain embodiments, a composition that includes suberin, cutin, or lignin for topical administration may be formulated to include between about 0.1% to 1%, between about 0.1% to 2%, between about 0.1% to 3%, between about 0.1% to 4%, between about 0.1% to 5%, between about 0.1% to 6%, between about 0.1% to 7%, between about 0.1% to 8%, between about 0.1% to 9%, between about 0.1% to 10%, 10% or more, 15% or more, 20% or more, 25% or more, or 30% or more of suberin, cutin, or lignin, by weight or volume of the composition.

Plant-derived biopolymers (suberin, cutin, lignin), have unique chemical structures with superhydrophilic, superhydrophobic, and polyphenolic structures. Because of these chemical properties, they are known to have antioxidant, anti-aging, anti-inflammatory, free-radical scavenger, and antimicrobial, antimutagenic/antitumor activities. (Santos et al. 2010; Gonçalves et al. 2016; Carriço et al. 2019; Carriço et al. 2018; Fernandes et al. 2009; Noferi et al. 1997; Krizková et al. 1999; Koch and Barthlott 2009). In addition, lignin also provides UV protection and may be used as a sunblock. (see Koch and Barthlott 2009; Qian et al. 2015). In addition, the biopolymers (suberin, cutin, lignin) with their unique physicochemical properties play a superior role in drug delivery across the skin making it ideal for dermal and transdermal drug delivery by increasing the drug permeability. This unique drug delivery capability of biopolymers (suberin, cutin, lignin) together with their antimicrobial, antitumor/anti-cancer, anti-inflammatory, antioxidant properties provides an opportunity for a greater efficacy of active drug, quicker onset of action and allows to use smaller concentration of the active drug and hence lowering their side effects. Biopolymers (suberin, cutin, lignin) also have their own antimicrobial (antiviral, antifungal/yeast, and antibacterial) properties. When combined with lysozyme, the biopolymers (suberin, cutin, lignin) have a unique capability to provide a greater stability to lysozyme degradation, which may provide an enhanced (synergistic and/or additive) antimicrobial response.

Consequently, cutin, suberin, lignin and other plant and cork extracts may provide several benefits to improve the appearance of skin including, but not limited to, improving hydration, reducing or preventing signs of aging, reducing or preventing wrinkles and fine lines, improving elasticity and/or tone of the skin, improving dull skin, tactile roughness, coarse wrinkles, skin atrophy, treating internal damage to the skin caused by exposure to UV radiation, improving firmness, reducing weathered appearance, smoothing of skin, whitening of skin, improving skin pigmentation. Cutin, suberin, lignin and other plant and cork extracts may also, provide wound healing, antimicrobial, anti-inflammatory, antimutagenic/antitumor, antioxidant, and free-radical scavenger properties. Cutin, suberin, lignin and other plant and cork extracts may also provide protection from environmental stress that causes skin damage including, but not limited to, pollution, hard water, surfactants, preservatives, perfumes, sun, wind, and high temperatures. The properties of suberin, lignin, and cutin can include providing or replacing a barrier function, preventing dehydration and protecting underlying cells in plants. Their use in pharmaceutical and cosmetic compositions described herein may provide a similar role when used on human skin, acting as an adjuvant when administered in conjunction with other agents and helping to improve the therapeutic efficacy of those agents, while also acting to condition the skin on their own. For example, cutin and/or suberin, and/or lignin may be used in conjunction with a retinoid, as discussed above. Skin irritation associated with retinoid therapy may be accompanied by an increase in TEWL. The use of suberin and/or cutin and/or lignin in conjunction with a retinoid acts to prevent or replenish the TEWL caused by retinoid therapy, thereby improving the barrier function of the skin and reducing or overcoming the side effects of retinoids.

For example, the suberin and/or cutin and/or lignin may be administered in conjunction with a retinoid to prevent or reduce a side effect associated with retinoid therapy. In some embodiment, the combination may be administered to prevent or reduce one or more of skin irritation, dryness, redness, epidermal hyperplasia, edema, blistering, crusting, erythema, burning, stinging, pruritus, heightened burning susceptibility upon exposure to sunlight, skin peeling, and overall physical discomfort. Administration of the second and/or third primary agent (e.g., suberin and cutin, or lignin) may aid in rendering the retinoid therapy more tolerable for the subject. This, in turn, may increase the likelihood that the subject will continue the retinoid therapy to achieve the retinoid's beneficial effects. Because suberin and/or cutin and/or lignin may increase tolerance to the retinoid, administration of the second and/or third primary agent may also aid in allowing a higher dose of the retinoid to be administered to the subject, which may result in a higher efficacy of the treatment and/or reduce the length of treatment needed to produce beneficial results and achieve a quicker onset of action.

Further, while plant-based biopolymers, astaxanthin, and arvelexin are used in the compositions described herein to reduce side effects and enhance compliance associated with treatment as discussed above (e.g., to improve TEWL, to reduce inflammation associated with the use of retinoids), each of those agents have their own anti-aging activities as well. Thus, the combination of plant-derived biopolymers, astaxanthin, and/or arvelexin with retinoids may provide additive or synergistic effects to potentiate the anti-aging properties of retinoids.

Alternatively, cutin and/or suberin and/or lignin may be used in the absence of a vitamin-A-based agent according to some embodiments. In such embodiments, the protective agent or agents may be used in a pharmaceutical or cosmetic composition on their own or in combination with one or more other primary agents described herein.

In some embodiments, the first and/or second and/or third primary agent of the composition is a lysozyme. Lysozyme, also known as muramidase or N-acetylmuramide glycanhydrolase, refers to a family of antimicrobial enzymes produced by animals that form part of the innate immune system. Lysozyme is a glycoside hydrolase that catalyzes the hydrolysis of 1,4-beta-linkages between N-acetylmuramic acid and N-acetyl-D-glucosamine residues in peptidoglycan, which is the major component of gram-positive bacterial cell wall. This hydrolysis in turn compromises the integrity of bacterial cell walls causing lysis of the bacteria. Lysozyme can be found in animal secretions such as tears, saliva, milk, and mucus, as well as in cytoplasmic granules of macrophages and polymorphonuclear neutrophils (PMNs). Another abundant source of lysozyme is in egg white. As used herein, a lysozyme may refer to naturally occurring lysozymes, recombinant lysozyme, synthetic lysozyme, or lysozyme salts. Compositions that include lysozyme may be used in dermatologic, ophthalmologic, gynecologic, surgical, or other medical or cosmetic settings to prevent or treat infections or other conditions such as folliculitis, impetigo, hidradenitis suppurativa, MRSA (Methicillin resistant Staphylococcus aureus), acne vulgaris, rosacea, acne and rosacea; as well as in applications such as wound care, burn wound care for first degree and second degree burn, third degree burn, and various lubricants. In some embodiments/compositions, lysozyme is also added as a preservative. In some embodiments, a composition that includes lysozyme for topical administration may be formulated to include between about 0.1% to 1%, between about 0.1% to 2%, between about 0.1% to 3%, between about 0.1% to 4%, between about 0.1% to 5%, between about 0.1% to 6%, between about 0.1% to 7%, between about 0.1% to 8%, between about 0.1% to 9%, between about 0.1% to 10%, between about 0.1% to 15%, between about 0.1% to 20%, between about 0.1% to 30%, between about 0.1% to 40%, between about 0.1% to 50%, of lysozyme, by weight or volume of the composition.

In some embodiments, the first and/or second and/or third primary agent of the composition is nisin. Nisin is a lantibiotic, which are a polycyclic antibacterial peptide produced by the bacterium Lactococcus lactis. Nisin is often used as a food preservative. While nisin has demonstrated activity against Gram-positive bacteria, it can be an effective inhibitor of certain Gram-negative bacteria by itself, and/or when used in combination with other compounds such as chelating agents. In some embodiments, nisin is added to compositions that include lysozyme to improve antibacterial efficacy, for example the combination of nisin and lysozyme may confer potent protection from gram-positive and gram-negative bacteria. In some embodiments/compositions, nisin is also added as a preservative. In some embodiments, a composition that includes nisin for topical administration may be formulated to include between about 0.1% to 1%, between about 0.1% to 2%, between about 0.1% to 3%, between about 0.1% to 4%, between about 0.1% to 5%, between about 0.1% to 6%, between about 0.1% to 7%, between about 0.1% to 8%, between about 0.1% to 9%, between about 0.1% to 10%, between about 0.1% to 15%, between about 0.1% to 20%, between about 0.1% to 30%, between about 0.1% to 40%, between about 0.1% to 50%, of nisin, by weight or volume of the composition.

According to some embodiments, a first primary agent, when administered in conjunction with a second and/or third primary agent may lead to an enhanced efficacy as compared to the use of the first primary agent alone. The enhanced efficacy can be additive, or it can be synergistic. Examples are discussed in detail below.

According to some embodiments, supplemental agents that may be included in the compositions described herein include, but are not limited to, an antibiotic or antibacterial agent, an antiseptic or biocide agent, an antiviral agent, an antimicrobial agent, an anesthetic, an antioxidant, an anti-wrinkle agent, an anti-acne agent, a vitamin, an anti-inflammatory agent, a keratolytic agent, an antifibrotic or anti-scar agent, an antineoplastic agent, a UV protection agent, a moisturizing agent, an essential oil, a thickening agent, a preservative, a natural or synthetic fragrance, or dye, or a flavoring agent, a deodorant, an antiperspirant, a skin lightening agent, pearlizing and/or opacifying agents, or a pharmaceutical agent. When administered in conjunction with the first agent and/or second and/or third agent, the supplemental agent or agents that are included may lead to an enhanced efficacy as compared to the use of the first agent, second agent, or third agent alone or in combination. The enhanced efficacy can be additive, or it can be synergistic. It can also enhance tolerability and hence increased compliance and improved clinical response.

Antimicrobial or antifungal agents in addition to plant-derived biopolymers (suberin, cutin and lignin), astaxanthin, or arvelexin (discussed above) that can be used in the compositions according to the embodiments described herein include, but are not limited to, coal or tar, sulfur, aluminum chloride, gentian violet, ciclopirox, octopirox (piroctone olamine), 3,4,4′-trichlorocarbanilide (trichlosan), triclocarban, ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline ciloquinol, thiobendazole, miconazole (1.0%, 2.0%, 3.0%, 4.0%), clotrimazole (1.0%, 2.0%), Nystatin (100,000 units/g; 100000 units/g; 100,000 units; 100,000 units/g with emollients), Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone 1 mg-1000 mg), thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, iodopropynyl butylcarbamate, azelaic acid, isothiazalinones such as octyl isothiazolinone and azoles, parabens (e.g., methylparaben, ethylparaben, etc.), glycols (e.g., hexylenglycol, ethylhexylglycerin), ketoconazole, Naftifine (1.0%-5.0%), hydrochloride, oxiconazole nitrate, sulconazole nitrate, urea, terbinafine hydrochloride, selenium sulfide, crotamiton, ivermectin Stromectol, permethrin, and polyvalent metal salts of pyrithione. In certain embodiments, the composition includes from about 0.001% to about 30%, from about 0.01% to about 10%, or from about 0.05% to about 5%, by weight or volume of the composition, of the antimicrobial agent(s). The exact content (%) of antimicrobial agent(s) to be used in the compositions will depend on the particular antimicrobial utilized since such agents vary widely in potency. For example, in some embodiments, the composition includes about 5% to about 20% of azelaic acid by weight or volume. In some embodiments, the composition includes about 5%, 6%, 7%, 8%, 9%, or 10% of ciclopirox by weight or volume. In some embodiments, the composition includes about 1%, 2%, 3%, 4%, or 5% of ketoconazole by weight or volume. In some embodiments, the composition includes between about 1% to about 15% (e.g., about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15%) of itraconazole by weight or volume. In some embodiments, the composition includes between about 1% to about 30% (e.g., about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, 20%, 21%, 22%, 23%, 24%, 25%, 26%, 27%, 28%, 29%, or 30%) of terbinafine by weight or volume.

Antibiotics (i.e., antibacterial agents) and other antimicrobial agents that can be used in the compositions according to the embodiments described herein include, but are not limited to, bacitracin, neomycin, polymyxin b, pramoxine, gentamycin, ozenoxacin, sulfacetamide sodium, silver sulfadiazine, retapamulin, sulfur, erythromycin, fusidic acid, mupirocin, pramoxine, mafenide, and tetracycline, Ovotransferrin Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin, minocycline, and clindamycin. In certain embodiments, the composition includes from about 0.001% to about 10%, from about 0.01% to about 5%, or from about 0.05% to about 2%, by weight or volume of the composition, of the antibiotic agent(s). The exact content (%) of antibiotic agent(s) to be used in the compositions will depend on the particular antibiotic utilized since such agents vary widely in potency.

Additional antimicrobials that can be used in the compositions disclosed herein include N-alkyl ethylbenzyl dimethyl ammonium chloride (C12-C14) and/or benzalkonium chloride at a concentration of between about 0.01 to about 2% by weight or volume (in some embodiments, the concentration of N-alkyl ethylbenzyl dimethyl ammonium chloride is about 0.05%, 0.154%, or 1.25% by weight or volume); and/or alcohol (e.g., ethanol, isopropanol) at a concentration of up to about 60%-70%, or about 70-80% by weight or volume. Such antimicrobials are commonly used as disinfectants or hand sanitizers in hospitals and other clinical health care facilities, as well as veterinary medicine and dental clinics/facilities.

Antiseptic or biocide agents that can be used in the compositions according to the embodiments described herein include, but are not limited to, chlorhexidine, triclosan, povidone-iodine, alcohols (ethyl alcohol, isopropyl alcohol, and n-propanol), and hydrogen peroxide. In certain embodiments, the composition includes from about 1% to about 80%, from about 1% to about 70%, from about 1% to about 80%, from about 1% to about 60%, from about 1% to about 50%, from about 1% to about 40%, from about 1% to about 30%, from about 1% to about 20%, or from about 1% to about 10%, by weight or volume of the composition, of the antimicrobial agent(s). The exact content (%) of antimicrobial agent(s) to be used in the compositions will depend on the particular antimicrobial utilized since such agents vary widely in potency.

Anesthetic agents that can be used in the compositions according to the embodiments described herein include, but are not limited to, benzocaine, lidocaine, bupivacaine, chlorprocaine, dibucaine, etidocaine, mepivacaine, tetracaine, dyclonine, hexylcaine, procaine, ketamine, pramoxine, phenol; and pharmaceutically acceptable salts thereof; benzyl alcohol, camphor, menthol, resorcinol; and appropriate combinations thereof. In some embodiments, the composition includes from about 0.001% to about 25%, from about 0.01% to about 10%, or from about 0.1% to about 5%, of an anesthetic, by weight or volume of the composition. The exact content (%) of anesthetic to be used in the compositions will depend on the particular anesthetic utilized since such agents vary widely in potency.

Antioxidants in addition to plant-derived biopolymers (suberin, cutin and lignin), astaxanthin, or arvelexin (discussed above) that can be used in the compositions according to the embodiments described herein include, but are not limited to, ascorbic acid (vitamin C) and its salts, ascorbyl esters of fatty acids, and other ascorbic acid derivatives (e.g., magnesium ascorbyl phosphate, sodium ascorbate, sodium ascorbyl phosphate, ascorbyl sorbate, ascorbyl palmitate, ascorbyl dipalmitate, ascorbyl stearate, ascorbyl methylsilanol pectinate, Betanin, Betalain, Betacyanins, Betaxanthins, magnesium ascorbate, disodium ascorbyl sulfate, tetrahexyldecyl ascorbate, etc.), ascorbic acid polypeptide, BHA, BHT, hydroquinone, t-butyl hydroquinone, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL). Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone), caffeine, cysteine, cysteine HCl, diamylhydroquinone, di-t-butylhydroquinone, tocopherol (vitamin E), tocopherol sorbate, tocopherol acetate, other esters of tocopherol, tocopheryl acetate, tocopheryl linoleate, tocopheryl nicotinate, Ovotransferrin, tocopheryl succinate, tris(nonylphenyl)phosphite, tocophereth-5, tocophereth-10, tocophereth-12, tocophereth-18, tocophereth-50, tocophersolan, dioleyl tocopheryl methylsilanol, potassium ascorbyl tocopheryl phosphate, beta-carotene, butylated hydroxy benzoic acids and their salts, ferulic acid, ethyl ferulate, peroxides including hydrogen peroxide, perborate, thioglycolates, sodium thioglycolate, thiodiglycol, thiodiglycolamide, thiodiglycolic acid, thioglycolic acid, thiolactic acid, thiosalicylic acid, dicetyl thiodipropionate, distearyl thiodipropionate, ditridecyl thiodipropionate, isooctyl thioglycolate, phenylthioglycolic acid, persulfate salts, potassium sulfite, sodium bisulfite, sodium metabisulfite, sodium sulfite, 6-hydroxy-2,5,7,8-tetramethylchroman-2-carboxylic acid, gallic acid and its alkyl esters, (e.g., propyl gallate, dodecyl gallate, octyl gallate), uric acid and its salts and alkyl esters, amines (e.g., N,N-diethylhydroxylamine, amino-guanidine), nordihydroguaiaretic acid, bioflavonoids, sulfhydryl compounds (e.g., glutathione), dihydroxy fumaric acid and its salts, lysine pidolate, arginine pilolate, amino acids, sodium erythorbate, quinones, rosmarinic acid, silymarin, lysine, 1-methionine, proline, superoxide dismutase, sorbic acids and its salts, sorbityl furfural, lipoic acid, olive extracts, tea extracts, resveratrol, polyphenols such as proanthocyanidine from pine bark, carotenoids, curcumin compounds such as tetrahydrocurcumin, kojic acid, coenzyme Q10, OCTA (L-2-oxo-4-thiazolidine carboxylic acid), selenium, creatine, glutathione, erythorbic acid, acetyl cysteine, N-acetyl cysteine, N-acetyl cysteine esters, dimethylmethoxy chromanol, lipoic acid, melanin; natural botanical anti-oxidants including but not limited to coffee berry extracts, green tea extracts, rosemary extracts, witch hazel extracts, and grape skin/seed extracts. In some embodiments, the composition includes from about 0.001% to about 25%, from about 0.01% to about 10%, or from about 0.1% to about 5%, of an anti-oxidant, by weight or volume of the composition. The exact content (%) of anti-oxidant to be used in the compositions will depend on the particular anti-oxidant utilized since such agents vary widely in potency.

Antiviral agents that can be used in the compositions according to the embodiments described herein, in addition to biopolymers (suberin, cutin, lignin), astaxanthin, or arvelexin, include, but are not limited to, acyclovir and penciclovir, famciclovir, ganciclovir, Quercetin (3,3′,4′5,7-Pentahydroxyflavone), Ovotransferrin. Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL). Equol (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM). In some embodiments, the composition includes from about 0.05% to about 1%, from about 0.05% to about 2.5%, or from about 0.5% to about 5%, of an antiviral agent, by weight or volume of the composition. The exact content (%) of antiviral agent to be used in the compositions will depend on the particular antiviral utilized since such agents vary widely in potency.

Anti-parasitic agents that can be used in the compositions according to the embodiments described herein include, but are not limited to, topical Permethrin 2.5%, and Permethrin 5%, topical Stromectol 1%, and oral Stromectol 3 mg, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Oramectin, Ivermectin, Eprinomectin, Eprimectin, Avermectin. Moxidectin. Abamectin. The topical products may be in the form of solution, lotion, gel, hydrogel, cream, ointment, foam, shampoo, cleanser, rinse, wipe. Parasitic diseases of skin include but not limited to scabies, head lice, crab lice (body lice). These parasitic infections cause characteristic inflammatory skin rash which is generally accompanied with intense pruritis lasting a long time even after the eruption (i.e. infestation) has resolved due to persistent inflammatory response of the host. The combination of astaxanthin, and/or arvelexin, and/or biopolymers such as suberin, cutin or lignin with anti-parasitic agent(s) can be used effectively as anti-inflammatory agents. However, their anti-pruritic properties when used as adjunct for treatment of various parasitic infections such as scabies, and for treatment in post-scabies and post-parasitic pruritis, instead of topical steroids, which have many undesirable side effects will provide an unexpected therapeutic advantage. These compositions, therefore, provide beneficial “steroid sparing” effects.

Vitamins that can be used in the compositions according to the embodiments described herein include, but are not limited to, vitamin A, vitamin C, vitamin C derivatives, ascorbic acid, ascorbyl glucoside, ascorbyl palmitate, magnesium ascorbyl phosphate, sodium ascorbyl phosphate, tetrahexadecyl ascorbate, ascorbyl 3-aminopropyl phosphate, vitamin B, vitamin B derivatives, vitamin B1 to vitamin B12 and their derivatives (e.g., vitamin B1, B2, B3, B5, B6, B7, B9, B12), vitamin K, vitamin K derivatives, vitamin H, vitamin D, vitamin D2, vitamin D3, vitamin D derivatives, vitamin E (Tocopherols), vitamin E derivatives, and pro-vitamins thereof, such as panthenol and mixtures thereof. The vitamin compounds may be included as the substantially pure material, or as an extract obtained by suitable physical and/or chemical isolation from natural (e.g., plant) sources. In some embodiments, the compositions contain from about 0.0001% to about 25%, from about 0.001% to about 10%, from about 0.01% to about 5%, or from about 0.1% to about 1%, by weight or volume of the composition, of the vitamin compound. The exact content (%) of vitamins to be used in the compositions will depend on the particular vitamin utilized since such agents vary widely in potency.

Anti-inflammatory agents in addition to plant-derived biopolymers (suberin, cutin and lignin), astaxanthin, or arvelexin (discussed above) that can be used in the compositions according to the embodiments described herein include, but are not limited to, steroidal, natural, and non-steroidal anti-inflammatory agents. In in some embodiments, the composition includes from about 0.01% to about 10%, or from about 0.5% to about 5%, of an anti-inflammatory agent by weight or volume of the composition. The exact content (%) of anti-inflammatory agent(s) to be used in the compositions will depend on the particular anti-inflammatory agent utilized since such agents vary widely in potency.

Non-limiting examples of steroidal anti-inflammatory agents include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, fluradrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, flucinonide, hydrocortisone valerate, hydrocortisone cyclopentylpropionate, hydrocortamate, meprednisone, paramethasone, prednisolone, prednisone, betamethasone, beclomethasone dipropionate, triamcinolone, Clobetasol propionate, and mixtures thereof.

Non-limiting examples of non-steroidal anti-inflammatory agents include diclofenac, indomethacin, oxicams such as piroxicam, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Equol (4′,7-isoflavandiol) (R-equol, racemic equol or S-equol (1%-25%, nM-25 nM, img, 2.5 mg, 5 mg, 7.5 mg, 10 mg, 15 mg, 20 mg, 25 mg, 30 mg, 40 mg, 50 mg-100 mg, or 1 nM-25 nM), Quercetin(3,3′,4′5,7-Pentahydroxyflavone), salicylates such as aspirin; acetic acid derivatives such as felbinac, fenamates such as etofenamate, flufenamic acid, mefenamic acid, meclofenamic acid, tolfenamic acid; propionic acid derivatives such as ibuprofen, naproxen, pyrazoles, and mixtures, salts, and esters thereof. Additional non-steroidal anti-inflammatory agents include allantoin and compounds of the Licorice, including glycyrrhetic acid, glycyrrhizic acid, and derivatives thereof (e.g., salts and suitable esters), diosgenol, saponines, sapogenines, lignanes, triterpenes saponosides and genines, anti-inflammatory interleukins (e.g., IL-1ra, IL-10); anti-inflammatory fatty acids (e.g., linoleic acid, linolenic acid) and their derivatives (e.g., esters), isoprenylcystein analogues (i.e., N-acetyl-S-famesyl-L-cysteine), aromatic aldehydes with anti-inflammatory properties (e.g., 4-methoxy benzaldehyde, 4-ethoxy benzaldehyde, 4-butoxy benzaldehyde, 4-penthoxy benzaldehyde), Ovotransferrin, as well as any compatible combinations thereof. Other non-steroidal anti-inflammatory agents include Voclosporin, tacrolimus 0.1% and 0.3% and pimecrolimus 1%, and sirolimus 0.5 mg, 1.0 mg, 2.0 mg orally, and 1.0 mg/ml oral solution.

Voclosporin, tacrolimus (0.1% and 0.3%) and pimecrolimus 1% are part of a class of drugs called “calcineurin inhibitors”, Both tacrolimus (0.1% and 0.3%) and pimecrolimus 1% approved by FDA for the treatment of atopic dermatitis and are also used by dermatologists for treatment of various inflammatory skin disorders such as psoriasis vulgaris, various forms of dermatitis/eczema, auto-immune skin diseases, immunosuppressive, and for the treatment of vitiligo both in pediatric and adult populations. Side effects of these topical medications include burning, stinging, itching, peeling and dryness at the skin application site which at times may limit their use. Biopolymers (cutin, suberin, lignin) and/or astaxanthin (or arvelexin) with their anti-inflammatory and antioxidant, and UV protection capabilities, as well as their potent moisturizing and wound healing properties, when combined with Voclosporin (various topical forms), tacrolimus (0.1% and 0.03%) or pimecrolimus 1%, provide a safe and effective novel alternative which will reduce side effects and enhance compliance with steroid spearing effect. Additionally, the combination products may provide an additive and synergistic anti-inflammatory response in treatment of atopic dermatitis and other forms of dermatitis, vitiligo, as well as other inflammatory skin conditions including, but not limited to, pyoderma gangrenosum, acute and/or chronic ulcer, pressure ulcer, chronic non-healing ulcers including but not limited to stasis dermatitis/stasis ulcer, diabetic foot ulcer, Behcet's disease, discoid lupus, lupus ulcer, various granulomatous skin/mucosal diseases such as necrobiosis lipoidica diabeticorum, in both in pediatric and adult populations. These products are in topical formulations according to the embodiments described herein including, but not limited to, emulsions, lotion, solution, cream, ointment, gel, hydrogel, shampoo, foam, wipe, spray, balm milk.

Topical Calcipotriene 0.005% (Dovonex)® a vitamin D analog, is a non-steroidal compound, with immunomodulatory properties approved by FDA for the treatment of psoriasis vulgaris. The main side effects of Calcipotriene include skin irritation, redness, burning, dryness or peeling of skin, and even worsening of psoriasis, which at times, leads to discontinuation of Calcipotriene. Alternatively, psoriasis vulgaris is typically treated with long-term application of topical steroids. The long-term use of topical steroids may have deleterious effects on skin which can limit their use. With their anti-inflammatory and antioxidant and UV protection capabilities of biopolymers (cutin, suberin, lignin) and/or astaxanthin (or arvelexin) as well as their potent moisturizing and wound healing properties, when combined with Calcipotriene, may provide a safe and effective novel alternative which will reduce its side effects and enhance compliance with steroid spearing effect. Additionally, the combination product may provide an additive and synergistic immune-mediated response in treatment of psoriasis both in pediatric and adult populations. These products are in topical formulations according to the embodiments described herein including, but not limited to, emulsions, lotion, solution, cream, ointment, gel, hydrogel, shampoo, foam, wipe, spray balm, milk.

Topical Calcipotriene 0.005% (Dovonex)® a vitamin D analog, is a non-steroidal compound, with immunomodulatory properties approved by FDA for the treatment of psoriasis vulgaris. Preparations containing topical Calcipotriene 0.005% is available by itself or in combination with different topical steroids. Due to side effects of prolong use of topical steroids, the use Calcipotriene 0.005% in combination with topical steroids is limited to short term use. The combination of Voclosporin (various topical forms), tacrolimus (0.1% and 0.03%) or pimecrolimus 1% which are non-steroid anti-inflammatory class of drugs called “calcineurin inhibitors with Calcipotriene 0.005%, may provide a safe and effective novel alternative which will eliminate the need for using topical steroids (steroid spearing), enhance compliance for long-term administration for the treatment of psoriasis vulgaris and for other inflammatory skin disorders. Additionally, the combination product(s) may provide an additive and synergistic immune-mediated response in treatment of psoriasis both in pediatric and adult populations. These products are in topical formulations according to the embodiments described herein including, but not limited to, emulsions, lotion, solution, cream, ointment, gel, hydrogel, shampoo, balm, milk, foam, wipe, spray.

Non-limiting examples of natural anti-inflammatory agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Betanin, Betalain, Betacyanins, Betaxanthins, kola extract, chamomile, red clover extract, sea whip extract, licorice extract, and tea extract, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone).

Anti-wrinkle agents (in addition to retinoids, plant-derived biopolymers (suberin, cutin and lignin), astaxanthin, and arvelexin as described above) that can be used in the compositions according to the embodiments described herein include, but are not limited to, amino acids, N-acetyl derivatives of amino acids (e.g., N-acetyl-cysteine), hydroxy acids (e.g., alpha-hydroxy acids such as lactic acid and glycolic acid or beta-hydroxy acids such as salicylic acid and salicylic acid derivatives such as the octanoyl derivative, lactobionic acid), Betanin, Betalain, Betacyanins, Betaxanthins, keto acids (e.g., pyruvic acid), phytic acid, ascorbic acid (vitamin C), kinetin (N6-furfuryladenine), zeatin and their derivatives (e.g., furfurylamino-tetrahydropyranyladenine), niacinamide (nicotinamide); Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone), caffeine; growth factors and cytokines (e.g., TGF-beta 1, 2 and 3, EGF, FGF-2, PDGF, IL-1, IL-6, IL-8, IGF-1, IGF-2, etc.), cell lysates (e.g., dermal fibroblast cell lysate, stem cell lysate, processed skin cell proteins (PSP®), etc.), conditioned cell culture mediums (e.g., conditioned cell culture medium from dermal fibroblasts, conditioned cell culture medium from stem cells (e.g., epidermal stem cells, adipose stem cells, mesenchymal stem cells, etc.), adenosine 5′-monophosphate (AMP) and/or adenosine 5′-monophosphate disodium salt (AMP2Na), cosmetic ingredients marketed under the trade names Nouricel-MD®, TNS®, or CCM™ Complex; etc.); cell extracts, stem cell extracts, components from stem cells; ingredients stimulating epidermal or other human adult stem cells; skin conditioning agents, stilbenes, cinnamates, ingredients activating sirtuin 1 (e.g., resveratrol); ingredients improving the functioning of the mitochondria; dimethylaminoethanol, synthetic anti-aging peptides, peptides from natural sources (e.g., soy peptides), and salts of sugar acids (e.g., Mn gluconate, Zn gluconate), lipoic acid; lysophosphatidic acid, vitamin E, vitamin B3 compounds, and other vitamin B compounds (e.g., thiamine (vitamin B1), pantothenic acid (vitamin B5), riboflavin (vitamin B2), and their derivatives and salts (e.g., HCl salts or calcium salts). In some embodiments, the compositions contain from about 0.0001% to about 25%, from about 0.001% to about 10%, from about 0.01% to about 5%, or from about 0.1% to about 1%, by weight or volume of the composition, of the anti-wrinkle agent. The exact content (%) of anti-wrinkle agent(s) to be used in the compositions will depend on the particular anti-wrinkle agent(s) utilized since such agents vary widely in potency.

Certain anti-aging agents are known to play a role in acceleration of epidermal renewal or rejuvenation of epidermis (i.e. epidermal turnover), which is typically delayed in aging of skin. The epidermal turnover is related to the energy metabolism of epidermal basal cells. Adenosine 5′-triphosphate (ATP) is needed for cell renewal and cell division; and adenosine 5′-monophosphate (AMP) and adenosine 5′-monophosphate disodium salt (AMP2Na) increase the amount of intracellular ATP. It is known that AMP accelerates the epidermal turnover delayed by aging (Furukawa et al. 2008). Thus, in certain embodiments, the compositions described herein may include adenosine 5′-monophosphate (AMP) and adenosine 5′-monophosphate disodium salt (AMP2Na) in combination with a composition that includes one or more of suberin, cutin, lignin, astaxanthin, and/or arvelexin (e.g., those compositions disclosed in the examples below).

Anti-acne agents that can be used in the compositions according to the embodiments described herein include, but are not limited to, resorcinol, sulfur, erythromycin, tetracycline, minocycline, clindamycin, salicylic acid, benzoyl peroxide, retinoic acid, tretinoin or other retinoids (as discussed above), alpha-hydroxy acids (e.g., glycolic acid, lactic acid), beta-hydroxy acids (e.g., salicylic acid), dehydroacetic acid, trichloroacetic acid, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL). Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone), and zinc. In some embodiments, the compositions contain from about 0.0001% to about 50%, from about 0.001% to about 20%, from about 0.01% to about 10%, or from about 0.1% to about 6%, by weight or volume of the composition, of the anti-acne compound. The exact content (%) of anti-acne agent(s) to be used in the compositions will depend on the particular anti-acne agent utilized since such agents vary widely in potency. For example, in some embodiments, an OTC anti-acne keratolytic agent may be used in the compositions described herein at a concentration of up to about 6% by weight or volume (e.g., between about 2% to 6) In one embodiment, the keratolytic agent is salicylic acid at a concentration of about 2%, 3%, 4%, 5%, or 6% by weight or volume.

Keratolytic agents that can be used in the compositions according to the embodiments described herein include, but are not limited to, suberin, lignin, cutin, ammonium lactate, urea, salicylic acid and alkyl derivatives thereof, podofilox, podophyllum resin, saturated and unsaturated monocarboxylic acids, saturated and unsaturated bicarboxylic acids, tricarboxylic acids, alpha hydroxyacids and beta hydroxyacids of monocarboxylic acids, alpha hydroxyacids and beta hydroxyacids of bicarboxylic acids, alpha hydroxyacids and beta hydroxyacids of tricarboxylic acids, ketoacids, alpha ketoacids, beta ketoacids, of the polycarboxylic acids, of the polyhydroxy monocarboxylic acids, of the polyhydroxy bicarboxylic acids, of the polyhydroxy tricarboxylic acids. Resorcinol and its low-molecular weight derivatives are other examples of useful keratolytic agents. In some embodiments, the compositions may include from about 0.01% to about 30%, about 0.1% to about 10%, or from about 0.5% to about 5%, to about 12%, to about 15%, to about 17.5%, to about 20% or higher of the keratolytic agent, by weight or volume of the composition. The exact content (%) of keratolytic agent(s) to be used in the compositions will depend on the particular desquamating/keratolytic agent utilized since such agents vary widely in potency. For example, in some embodiments, urea may be used in the compositions described herein at an OTC concentration of between about 5% to about 20% (e.g., about 5%, 7.5%, 10%, 12.5%, 15%, 17.5%, or 20%) by weight or volume; or at a prescription concentration of above about 20%, 30%, 35%, 37%, 39%, 40%, 50%, 60%, 70%, or above 70% by weight or volume. In other embodiments, ammonium lactate may be used in the compositions described herein at a concentration of between about 10% to 20% (e.g., about 12%, 13%, 14%, 15%, 17.5%, or 20%) by weight or volume.

Antifibrotic or anti-scar agents that can be used in the compositions according to the embodiments described herein include, but are not limited to, Nintedanib, Defactinib, Dasatinib, Nilotinib, Verteporfin, Pirfenidone, Silicone, and 5-fluorouracil. In some embodiments, the compositions may include up to 25% of the antifibrotic or anti-scar agent or agent, by weight or volume of the composition. The exact content (%) of antifibrotic or anti-scar agent(s) to be used in the compositions will depend on the particular antifibrotic or anti-scar agent(s) utilized since such agents vary widely in potency. For example, in some embodiments, an antifibrotic or anti-scar agent may be used in the compositions described herein at a concentration of between about 0% to about 25% (e.g., about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%), between about 0% to 5%, between about 5% to about 10%, between about 10% to about 15%, between about 15% to about 20%, between about 20% to about 25% by weight or volume. In other embodiments, the antifibrotic or anti-scar agent may be used in the compositions described herein at any known OTC or prescription strength.

Antineoplastic agents in addition to plant-derived biopolymers (suberin, cutin and lignin), astaxanthin, or arvelexin (discussed above) that can be used in the compositions according to the embodiments described herein include, but are not limited to, 5-fluorouracil (5-FU), imiquimod, ingenol, diclofenac, and mechlorethamine, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Betanin, Betalain, Betacyanins, Betaxanthins, Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone). In some embodiments, the compositions may include from about 0.01% to about 0.02%, about 0.01% to about 0.05%, about 0.1% to about 2.5%, about 0.1% to about 5%, or from about 0.01% to about 7.5%, or from 0.01% to 10% of the antineoplastic agent, by weight or volume of the composition. The exact content (%) of antineoplastic agent(s) to be used in the compositions will depend on the particular desquamating/keratolytic agent utilized since such agents vary widely in potency.

UV protection agents in addition to plant-derived biopolymers (suberin, cutin and lignin), astaxanthin, or arvelexin (discussed above) that can be used in the compositions according to the embodiments described herein include, but are not limited to, physical and chemical sunblock agents. Non-limiting examples of chemical sunblock agents include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate, ethylhexyl methoxycinnamate, and ethyl methoxycinnamate), cinnamate esters, salicylates (homomethyl salicylate, benzyl salicylate, ethylhexyl salicylate glycol salicylate, isopropylbenzyl salicylate, etc.), anthranilates, ethyl urocanate, homosalate, octisalate, dibenzoylmethane derivatives (e.g., avobenzone, butyl methoxydibenzoylmethane), Octocrylene, octyl triazone, digalloy trioleate, glyceryl aminobenzoate, lawsone with dihydroxyacetone, ethylhexyl triazone, dioctyl butamido triazone, diethylhexyl butamido triazone, benzylidene malonate polysiloxane, terephthalylidene dicamphor sulfonic acid, disodium phenyl dibenzimidazole tetrasulfonate, phenylbenzimidazole sulphonic acid, diethylamino hydroxybenzoyl hexyl benzoate, bis diethylamino hydroxybenzoyl benzoate, bis benzoxazoylphenyl ethylhexylimino triazine, benzimidazilate, anisotriazine, drometrizole trisiloxane, methylene bis-benzotriazolyl tetramethylbutylphenol, and bis-ethylhexyloxyphenol methoxyphenyltriazine, 4-methylbenzylidene camphor, benzophenone-5,4-methylbenzylidene camphor, and isopentyl 4-methoxycinnamate, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL). Equol (4′,7-isoflavandiol) (R-equol, racemic equol or S-equol (1%-25%, nM-25 nM, img, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone). Non-limiting examples of physical sunblocks include kaolin, talc, petrolatum and metal oxides (e.g., titanium dioxide and zinc oxide). Compositions of the present invention can have UVA and UVB absorption properties. The compositions can have a sun protection factor (SPF) of 2, 3, 4, 5, 6, 7, 8, 9, 10, 11, 12, 13, 14, 15, 20, 25, 30, 35, 40, 45, 50, 55, 60, 70, 80, 90, 100 or more, or any integer or derivative therein. In some embodiments, the composition contains from about 0.1% to about 25%, or from about 0.5% to about 10% by weight or volume of the composition, of the UV protective agent. Exact amounts will vary depending upon the sunscreen chosen and the desired Sun Protection Factor (SPF).

Moisturizing agents in addition to plant-derived biopolymers (suberin, cutin and lignin), astaxanthin, or arvelexin (discussed above) that can be used in the compositions according to the embodiments described herein include, but are not limited to emollients, humectants, and skin conditioners. In some embodiments, the composition includes from about 0.01% to about 80%, from about 0.1% to about 25%, or from about 0.5% to about 10%, or the moisturizing agent(s) by weight or volume of the composition. The exact content (%) of emollients, humectants, and conditioning agents to be used in the compositions will depend on the emollients, humectants, and conditioning agents utilized since such agents vary widely in potency.

Non-limiting examples of humectants, in addition to suberin, lignin, cutin, include polyhydric alcohols (e.g., polyhdroxy alcohols and glycerin, hexylene glycol, ethoxylated glucose, 1,2-hexane diol, dipropylene glycol, trehalose, diglycerin, maltitol, cetyl alcohol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof), water soluble alkoxylated nonionic polymers (e.g., polyethylene glycols and polypropylene glycols having a molecular weight of up to about 1000 such as those with CTFA names PEG-200, PEG-400, PEG-600, PEG-1000, and mixtures thereof), acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed proteins, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium pyrollidone carboxylic acid (PCA), propylene glycol, butylene glycol, sodium pyrollidone carboxylic acid (PCA), sorbitol, sucrose, dextran sulfate (i.e., of any molecular weight), Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), Persea gratissima (avocado), avocado oil, natural moisturizing factors, and/or urea.

Non-limiting examples of skin and hair conditioners include guanidine, urea, glycolic acid, glycolate salts (e.g., ammonium and quaternary alkyl ammonium), salicylic acid, lactic acid, lactate salts (e.g., ammonium and quaternary alkyl ammonium), aloe vera in any of its variety of forms (e.g., aloe vera gel), polyhydroxy alcohols such as sorbitol, mannitol, xylitol, erythritol, hexanetriol, butanetriol, propylene glycol, butylene glycol, hexylene glycol and the like, polyethylene glycols, propoxylated glycerols, sugars (e.g., melibiose), starches, sugar and starch derivatives (e.g., alkoxylated glucose, fructose, glucosamine), C1-C30 monoesters and polyesters of sugars and related materials, hyaluronic acid, lactamide monoethanolamine, acetamide monoethanolamine, panthenol, dexpanthenol, allantoin, and mixtures thereof. Skin and hair conditioners can also include fatty acids, fatty acid esters, lipids, ceramides, cholesterol, cholesterol esters, bee wax, petrolatum, and mineral oil.

Non-limiting examples of emollients, in addition to suberin, lignin, cutin, include acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, glycol esters, alkyl lactates, caprylyl glycol, cetyl esters, cetyl laurate, cetyl alcohol, coconut oil polyethylene glycol-10 esters, alkyl tartrates, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, palm oil, coconut oil, myreth-3 palmitate, octyldecanol, octyldodecanol, Odontella aurita oil, 2-oleamido-1,3 octadecanediol, palm glycerides, polyethylene glycol avocado glycerides, polyethylene glycol castor oil, Persea gratissima (avocado), avocado oil, polyethylene glycol-22/dodecyl glycol copolymer, polyethylene glycol shea butter glycerides, phytol, raffinose, stearyl citrate, sunflower seed oil glycerides, petrolatum, silicon oils including but not limited to caprylyl methicone, and/or tocopheryl glucoside.

Essential oils that can be used in the compositions according to the embodiments described herein include, but are not limited to, sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang. Over examples of oils that can be used include certain plant oils (e.g., olive oil, olive pomace oil, sunflower seed oil, coconut oil, Persea gratissima (avocado), avocado oil, safflower seed oil, argan oil, soybean oil, peanut oil, avocado oil, borage oil, jojoba oil, oat oil, pomegranate seed oil, almond oil, bitter apricot oil, rose hip oil, German chamomile oil, and shea butter, walnut oil, Japanese walnut oil, Heartnut (Juglans ailantifolia). Other essential oils known to those of skill in the art are also contemplated as being useful within the context of the present invention.

Thickening agents that can be used in the compositions according to the embodiments described herein include, but are not limited to, carboxylic acid polymers (e.g., crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol, carbomers), crosslinked polyacrylate polymers (e.g., cationic and nonionic polymers), polyacrylamide polymers (e.g., nonionic polyacrylamide polymers including substituted branched or unbranched polymers, polyacrylamide, isoparaffin and laureth-7, multi-block copolymers of acrylamides and substituted acrylamides with acrylic acids and substituted acrylic acids), polysaccharides (e.g., cellulose and derivatives thereof, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, hydroxyalkylated cellulose, lignin, cutin, suberin, microcrystalline cellulose, sodium cellulose sulfate, scleroglucans, and mixtures thereof), and gums (e.g., acacia, agar, algin, alginic acid, cetyl alcohol, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof).

Non-limiting example of penetration enhancers that can be used in the compositions, in addition to suberin, lignin, cutin, include dimethyl sulfoxide, ethanol, propylene glycol, glycerin, PEG, urea, dimethyl acetamide, sodium lauryl sulfate, poloxamers, Spans, Tweens, lecithin, and/or terpenes amongst others thereof.

Preservatives that can be used in the compositions, in addition to lysozyme and nisin, according to the embodiments described herein include, but are not limited to, quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal, and other natural preservatives, or combinations thereof.

Skin lightening (or whitening) agents in addition to plant-derived biopolymers (suberin, cutin and lignin), astaxanthin, or arvelexin (discussed above) that can be used in the compositions according to the embodiments described herein include, but are not limited to, ascorbic acid and derivatives thereof; kojic acid and derivatives thereof; resorcinol and derivatives thereof (including but not limited to 4-ethyl resorcinol, 4-butyl resorcinol, 4-hexyl resorcinol, 4-octyl resorcinol, 4-decyl resorcinol, 6-methyl resorcinol, 6-ethyl resorcinol, 6-butyl resorcinol, 6-hexyl resorcinol, 6-octyl resorcinol, 6-decyl resorcinol, 4-phenylethyl resorcinol), lignin peroxidase, retinoic acid and derivatives thereof (e.g., retinol, retinyl palmitate), L-leucine and derivatives thereof (e.g., N-acyl derivatives of L-leucine, esters of L-leucine, etc.), glycine and derivatives thereof, disodium glycerophosphate and derivatives thereof, undecenoyl phenylalanine, arbutin and derivatives thereof (e.g., dehydroxyarbutin), niacinamide and derivatives thereof, hydroquinone (at a concentration of between about 1-8%, or between about 2-4% by weight or volume; e.g., about 1%, 2%, 3%, 4%, 5%, 6%, 7%, or 8%); mequinol, glabridin, aleosin, curcumin, genistein, ethyl linoleate, tranexaminic acid, azelaic acid (1%-20%), resveratrol and derivatives thereof (e.g., oxyresveratrol), N-acetyl glucosamine, 4-isopropylcetchol, 4-ethoxybenzaldehyde, 2-ethoxybenzaldehyde, 4-propoxybenzaldehyde, alpha-hydroxyacids (e.g., glycolic acid, lactic acid, trichloroacetic acid, etc.), salicylic acid, Pterostilbene or its derivatives (trans-3,5-dimethoxy-4-hydroxystilbene) (0.1%-100%, 1 mg-1000 mg, 1.0 μM-500 μM, 1.0 μg/mL-1000 μg/mL), polyphenols; and/or various plant extracts, such as those from licorice, grape seed, mulberry, soy, green tea, and/or bear berry; and/or any ingredient or combination thereof. In some embodiments, the compositions include from about 0.01% to about 15%, from about 0.01% to about 20%, from about 0.1% to about 10%, or from about 0.5% to about 5%, by weight or volume of the composition, of a skin lightening agent. The exact content (%) of skin lightening agents to be used in the compositions will depend on the particular skin lightening agent utilized since such agents vary widely in potency.

Other pharmaceutical agents that can be used in the compositions according to the embodiments described herein include, but are not limited to, agents used to treat rosacea, analgesics, anesthetics, anorectals, antihistamines, anti-cancer agents or chemotherapeutics, anti-inflammatory agents, anti-microbial agents, antiparasitic, scabicides, pediculicides, antiperspirants, deodorants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, canker sore treatment agents, cold sore treatment agents, dental and periodontal treatment agents, photosensitizing actives, skin protectant/barrier agents, steroids including hormones and corticosteroids, sunburn treatment agents, transdermal actives, nasal actives, vaginal actives, wart treatment agents, wound treatment agents, wound healing agents, chelating agents (e.g., Ethylenediaminetetraacetic acid (EDTA)), and surfactants (e.g., Sodium stearate, 4-(5-dodecyl) benzenesulfonate, docusate (dioctyl sodium sulfosuccinate), alkyl ether phosphates, benzalkonium chloride (BAC), and perfluorooctanesulfonate (PFOS)).

Another supplemental agent that may be used in accordance with the embodiments described herein is lycopene, which is a carotenoid hydrocarbon with antioxidant, anti-inflammatory, anti-tumor, photoprotective, free radical scavenger, antimicrobial, antiaging properties.

Another supplemental agent that may be used in accordance with the embodiments described herein is squalene. Squalene on its own isn't stable enough for use in products intended to be kept on the shelves for some time, like skin care products, but in combination with one or more of the primary agents, its stability may be improved. Alternatively, the supplemental agent that may be used is squalene. Squalane is derived from squalene, and is the more stable form of this molecule, as well as being colorless and odorless. Squalane (or squalene) may be used as an emollient, to increase skin hydration and reduce water loss, and is excellent for barrier deficient skin; it may also be used for antioxidation, acts against free radicals, has antitumor activities, and because Lipid emulsions are a potentially effective drug delivery system, it may also act as a vehicle (in accordance with the disclosure below).

According to the embodiments described herein, the compositions may also include a vehicle. In some embodiments, the composition includes a vehicle suitable for a topical or oral administration. Non-limiting examples of suitable vehicles, in addition to suberin, lignin, cutin, include aerosols and sprays, anhydrous bases in solid form (e.g., powder, sticks, lipsticks), creams, dispersions, drops, syrups, emulsions (e.g., water-in-oil, water-in-oil-in-water, oil-in-water, silicone-in-water, water-in-silicone, oil-in-water-in-oil, oil-in-water-in-silicone emulsions), foams, gels, hydrogel, milks, liposomes, lotions (e.g., suspensions), microsponges, multi-phase systems, nanoparticles, ointments, pastes, semi-solid forms, wipes, serums, solutions (including aqueous, non-aqueous, hydro-alcoholic, water-based, oil-based, lubricants, tinctures, colloids, liniments, paints,), transdermal patches, tablets, capsules, time-release or sustained release tablets or capsules, chewable tablets, dissolvable tablets, lozenges, pledget, granules, vesicles, or by other method or any combination of the forgoing as would be known to one of ordinary skill in the art. Variations and other appropriate vehicles will be apparent to the skilled artisan and are appropriate for use in the compositions described herein. In certain aspects, it is important that the concentrations and combinations of the compounds, ingredients, and agents be selected in such a way that the combinations are chemically compatible and do not form complexes which precipitate from the finished product.

Non-limiting examples of vehicle ingredients include emollients (e.g., acetyl alcohols, stearyl alcohol, stearic acid, isopropyl palmitate, squalene, lanolin, glycerin, petrolatum, petroleum), humectants (e.g., propylene glycol, sorbitol, glycerin), thickening agents (e.g., bee wax, xanthum gum, petrolatum), solvents (e.g., water, and/or organic solvents such as ethanol, glycerin, propylene glycol, butylene glycol, other alcohols, ether, acetone), emulsifying agents, penetration enhancers (e.g., chemical enhancers like propylene glycol, urea, DMSO; physical enhancers like iontophoresis, phonophoresis, sonophoresis, mircodermobrasion, microneedles), binders, disintigrant, coating, and stabilizers (e.g., parabens, halogenated phenol, sodium benzoate, propylene glycol, thiomersol), biopolymers (suberin, cutin, lignin).

In some embodiments, the vehicle may include a solvent present at about 20% to about 99%, or from about 60% to about 90% by weight or volume of the composition. The solvent may contain water, or a miscible mixture of water and organic solvent (e.g., alcohols, including but not limited to ethanol, glycerin, propylene glycol, butylene glycol, other glycols, etc.), but may alternatively contain water with significant lower or no concentrations of organic solvent, except as otherwise incidentally incorporated into the composition as minor ingredients of other essential or optional components.

In some embodiments, the vehicle is an emulsion. In some embodiments, the emulsion may contain an aqueous phase and a lipid or oil. Lipids and oils may be derived from animals, plants, or petroleum and may be natural or synthetic (e.g., man-made). In certain embodiments, the emulsion may include a humectant (e.g., propylene glycol, sorbitol, glycerin). In certain embodiments, the emulsion may include from about 0.1% to about 25%, or from about 0.2% to about 10%, of an emulsifying agent (emulsifier), based on the weight of the composition. Emulsifier agents may be nonionic, anionic, or cationic, and are selected based on the properties of the ingredients to be mixed. For example, the emulsifier may be a water-in-oil emulsifier (W/O), an oil-in-water emulsifier (0/W), a water in silicone emulsifier (W/Si), a liquid emulsifier, a solid emulsifier, an instant cold emulsifier, or an emulsifier for sprays. Suitable emulsifiers may include, but are not limited to glyceryl monostearate, PEG 7 glyceryl cocoate, glycol stearate, glycol distearate, PEG-150 distearate, lecithin, hydrogenated lecithin, sodium cocoyl glycinate, 12-hydroxy stearic acid, hydrogenated castor oil, cetyl alcohol, ceto stearyl alcohol, cetearyl alcohol, stearyl alcohol, behenyl alcohol, lauric acid, myristic acid, palmitic acid, stearic acid, oleic acid, polysorbates (e.g., polysorbate 20, polysorbate 60, polysorbate 80), ceteareth-25, ceteareth-20, glyceryl stearate, glyceryl stearate citrate, sucrose stearate, shea butter glycerides, polyglyceryl oleate, sorbitan stearate, glyceryl oleate, sorbitan oleate, polyglyceryl oleate, gum Arabic, isopropyl palmitate, laureth-3, PEG-8 beeswax, squalene, lanolin, glycerin, petrolatum, petroleum, biopolymers (suberin, cutin, lignin). Emulsions may have a wide range of viscosities, depending on the desired product form. The compositions of the present invention can be in the form of pourable liquids, semi-solids, to highly viscous systems (e.g., solids) under ambient conditions.

In some embodiments, the vehicle may be a solution, lotion, serum, hydrogel, or cream. In such embodiments, the solution, lotion, serum, or cream may contain an acceptable emollient present at about 1% to about 80% of the composition by weight or volume. As used herein, “emollient” refers to a material useful for the prevention or relief of dryness, as well as for the protection of the skin. A wide variety of suitable emollients is known and may be used herein. Suitable emollients may include, but are not limited to, acetyl alcohols, stearyl alcohol, stearic acid, isopropyl palmitate, squalene, lanolin, glycerin, petrolatum, petroleum, and other emollients discussed herein. In one embodiment, the emollient is glycerin. Glycerin may be used in an amount of from or about 0.001% to or about 80%, from or about 0.01% to or about 25%, from or about 0.1% to or about 10%, or from or about 2% to or about 5%.

In some embodiments, the vehicle may be a lotion, serum, hydrogel, or cream. In such embodiments, the lotion, serum, or cream may include a solvent and one or more emollients. In certain embodiments, a lotions or creams may contain from about 1% to about 50%, or about 1% to about 20%, of emollient; from about 50% to about 90%, or about 60% to about 80%, water; and, optionally, additional substances in amounts sufficient to provide additional benefits. Creams are generally thicker than lotions and serums due to higher levels of emollients, higher levels of thickeners, and/or differences in the emulsifying system.

In some embodiments, the vehicle is an ointment. In such embodiments, the ointment may contain one or more of (i) a simple base of animal, vegetable, and/or essential oils or semi-solid hydrocarbons such as petroleum or petrolatum; (ii) absorption ointment bases which absorb water to form emulsions; and (iii) water soluble solvent, e.g., a water soluble solution solvent. In some embodiments, the ointment may further contain a thickening agent and/or an emollient. For example, in some embodiments, an ointment may contain from about 2% to about 10% of an emollient by weight or volume; from about 0.1% to about 2% by weight or volume of a thickening agent as well as one or more optional additional substances(s) in amounts sufficient to provide additional benefits. The compositions described herein may be used in any suitable dermatological, cosmetic, or pharmaceutical product. Examples of products in which the compositions may be used according to some embodiments include, but are not limited to, anti-aging products, antidandruff products, anti-sweat and antiperspirant products, deodorant products, astringents, balms, bandages, body scrubs, brush-on products, antibacterial, antifungal, disinfectants, cleansers, colloids, cosmetics or make-up products (e.g., concealer sticks, compacts, eyeliner, blush, foundation, facial or body powder, lip balm, lip gloss, lip liner, lipstick, milk, mascara, masks, rouge), creams (e.g., cold creams, cream rinses, cream body wash, moisturizing creams, facial creams, daytime creams, night creams, tinted creams, and other skin creams), solution, wipes, sticks, masks, under-occlusion, pellets, troches, lozenges, pledgets, depilatories and permanent waxing solutions, dispersions, exfoliants, feminine products (e.g., vaginal product, vulvar products), finger nail products (e.g., nail conditioner, nail polish, nail polish remover), gels (e.g., bath gel, shower gel, skin gel, antibacterial hand gels, styling gels, gel soap, and other cleansing gels, hydrogels), hair-care products (e.g., hair dyes and coloring products, styling gels, hair sprays, mousse, shampoos, conditioners, leave-in conditioners, and other hair products), hygiene products, liquids, lotions (e.g., body lotions, face lotions, day lotions, night lotions, preshave or aftershave lotions, sunscreen lotions, and other lotions with skin benefits), macro-emulsions, medical devices, micro-emulsions, moisturizers, nano-emulsions, ointments, oral care products (e.g., mouthwash, toothpaste), chewing gum, pastes, patches (e.g., iontophoretic patches, microneedle patches, microprojection patches, transdermal patches), pencils, personal care product, powders, preshaving and after shaving products, rinses, serums, shaving, skin conditioner, skin delivery enhancing systems, skin ointment, skin product, skin rejuvenation product, skin-whiteners/brighteners, skin wipes, soaps (e.g., liquid soap, bar soap, gel soap, foam soap, or other soap-based cleansers), softeners, solids, shampoo, solutions, spray and aerosol products, sunblocks, sunless skin tanning products, sunscreens, suspensions, tissue cloths, tissue wipe, toners, topical medicaments. In some embodiments, products in which the compositions may be used include liquid soap, shower/bath products, hair color products, color-enhancing products, color-altering products, odor-altering products, sun protection products, after sun products, body care products, make up removal products, dark circles/under eye products, intimate cleansers, pre-shampoo scalp, hair mask, hair oil, curl cream, leave in conditioner, dry shampoo, hand sanitizer, all-purpose sanitizers (with and without 60-80% alcohol), antiseptic/antimicrobials/sanitizers used in clinical settings/hospital/surgical units, veterinary medicine/clinics settings, contact sports settings, intimate contact, wound care, lubricants (with and without anti-microbials), antiseptics, pre- and post-operative antiseptics, pre-biopsy anti-sceptics, post-biopsy wound care and dressing. Due to the agents' anti-oxidative properties, the hair product compositions discussed above can be used to protect, delay and to prevent oxidation of hair dye/coloring. Also, in the form of a spray, gel, etc. may be used to protect, delay and to prevent oxidation of hair dye because of certain agents' UV blocking properties (as discussed above).

In some embodiments, the compositions further comprise any additional additive commonly used in the field of application envisaged and the additives necessary for their formulation such as solvents, thickeners, diluents, antioxidants, colorants, sunscreens, self-tanning agents, pigments, fillers, preservatives, fragrances, odor absorbers, cosmetic or pharmaceutical actives, essential oils, vitamins, essential fatty acids, surfactants, film forming polymers, etc. In some embodiments, the compositions include pearlizing and/or opacifying agents, which are used in various skin care products such as shampoo, liquid hand soaps, shaving preparations and shower and bath products, lipsticks, etc. Pearlizing agents provide opacity and a creamy, rich, luxurious texture appearance to the products into which they are incorporated and help in masking the presence of other additives such as anti-dandruff agents and polymeric conditioners in shampoos. For example, the strong red or orange color of astaxanthin can be tempered by the pearlizing/opacifying agent (i.e. cutin and/or titanium oxide). In some embodiments, the pearlizing and/or opacifying agents are solid materials such as glycol distearate, stearic acid, cutin, and glyceryl stearate, titanium oxide.

In one embodiment, cutin may be used as a pearlizing agent to temper the strong red or orange color of astaxanthin. In certain aspects, a limiting factor for using doses of astaxanthin higher than 3% is its strong red color. Thus, in such embodiments, the use of cutin in combination with astaxanthin in cosmetic compositions may allow a higher dose of astaxanthin to be used, resulting in a more effective product.

In some embodiments, a primary agent, a supplemental agent, or an ingredient related to the vehicle may have more than one beneficial property, thus a single agent may fall into more than one of the categories of agents. Further, the terms “first” primary agent, “second” primary agent, “third” primary agent, etc. are not meant to be limiting with respect to order of importance or any other characteristic. The terms first, second, third, etc. are interchangeable in accordance with the embodiments described herein.

Compositions Including a Retinoid and/or a Plant-Derived Biopolymer

In some embodiments, the dermatological composition includes a retinoid as a first primary agent and cutin as a second primary agent. In some embodiments, the dermatological composition includes a retinoid as a first primary agent and suberin as a second primary agent. In some embodiments, the dermatological composition includes a retinoid as a first primary agent and lignin as a second primary agent. In some embodiments, the dermatological composition includes a retinoid as a first primary agent, cutin as a second primary agent, and suberin as a third primary agent. In some embodiments, the dermatological composition includes a retinoid as a first primary agent, cutin as a second primary agent, and lignin as a third primary agent. In some embodiments, the dermatological composition includes a retinoid as a first primary agent, lignin as a second primary agent, and suberin as a third primary agent. In some embodiments, the dermatological composition includes a retinoid as a first primary agent, and one or more of the supplemental agents. Such embodiments may optionally include one or more supplemental agents, and/or a vehicle such as one or more supplemental agents, and/or vehicles discussed in detail above.

As discussed above, compositions that combine retinoids with cutin, lignin, and/or suberin are advantageous to improve or maintain the skin's barrier function during retinoid treatment to reduce side effects associated with retinoid treatment, to improve efficacy of the retinoids (which may shorten treatment time), improve moisture retention during retinoid treatment, to improve user tolerance of the retinoid, and to improve patient compliance with recommended treatment. In certain embodiments, the combination of retinoids with cutin, lignin, and/or suberin also allows for a higher tolerance to higher dosages of retinoids, for improving the treatment for conditions that are generally very difficult to treat, and to achieve a quicker therapeutic effect.

In accordance with the embodiments described herein, compositions that include a retinoid may be used to treat or prevent any one or more of the following conditions: acne (e.g., acne vulgaris, cystic acne, acne conglobata, and acne rosacea), warts, condyloma, flat warts (verruca plana), plantar warts, verruca vulgaris, molluscum contagiosum, bowenoid papulosis, dyshidrotic eczema (e.g., eczema of the hands and feet), psoriasis, seborrheic dermatitis, atopic dermatitis, diaper dermatitis, aged skin (e.g., photoaged skin or chronologically aged skin), skin cancer, hyperpigmentation, cutaneous lesions of AIDS-related Kaposi's sarcoma, keratinization disorders (e.g., ichthyosis, Pityriasis rubra pilaris, and Darier's disease, porokeratosis, pitting keratolysis, keratosis pilaris, seborrheic keratosis), palmoplantar hyperkeratosis, xerosis, plantar hyperkeratosis, acanthosis nigricans, carcinoma (e.g., basal cell and squamous cell carcinomas, cutaneous T-cell lymphoma (mycosis fungoides), precancerous actinic keratosis), keratoacanthoma and cutaneous sarcoidosis, hidradenitis suppurativa, wrinkles, aging related skin changes, abnormal or diminished synthesis of collagen and elastin, diminished levels of collagen, shallowness, and elastin in the dermis, skin atrophy, Anetoderma, stretch marks (striae), skin lines, fine lines, thinning of skin, coarse wrinkles, tactile roughness, skin thickening due to elastosis of photoaging, loss or reduction of skin resiliency, elasticity and recoilability, lack of skin resiliency, elasticity, nodular elastoidosis, and recoilability, and older looking skin. Such compositions are also used to treat pigmentary skin changes such as freckles, solar lentigo, ephelides, and melasma as well as corns and callosities.

Compositions Including a Retinoid and/or Astaxanthin (with or without a Plant-Derived Biopolymer)

In some embodiments, the dermatological composition includes a retinoid as a first primary agent and astaxanthin as a second primary agent. In some embodiments, the dermatological composition includes a retinoid as a first primary agent, astaxanthin as a second primary agent, and cutin or suberin or lignin as a third primary agent. In some embodiments, the dermatological composition includes a retinoid as a first primary agent, astaxanthin as a second primary agent, cutin as a third primary agent, and suberin or lignin as a fourth primary agent. Such embodiments may optionally include one or more supplemental agents, and/or a vehicle such as one or more supplemental agents, and/or vehicles discussed in detail above.

In addition to the advantages discussed above with respect to compositions that include a retinoid and cutin, lignin and/or suberin, the compositions may also include astaxanthin. Astaxanthin's antioxidant, anti-inflammatory, and anti-UV properties can potentiate the effects of the retinoid, conferring an additional advantage for such compositions. In addition, astaxanthin has anti-tumor properties, which give an additional layer of protection for subjects experiencing UV sensitivity or photosensitization associated with retinoid treatment and provides protection against UV-induced mutation causing precancer growths and skin cancers.

In accordance with the embodiments described herein, compositions that include a retinoid and astaxanthin may be used to treat or prevent any one or more of the following conditions: acne (e.g., acne vulgaris, cystic acne, and acne rosacea, acne conglobate, warts, condyloma, flat warts (verruca plana), plantar warts, verruca vulgaris, molluscum contagiosum, bowenoid papulosis, dyshidrotic eczema (e.g., hyperkeratotic eczema of the hands and feet), psoriasis, seborrheic dermatitis, atopic dermatitis, aged skin (e.g., photoaged skin or chronologically aged skin), skin cancer, post-inflammatory hyperpigmentation, cutaneous lesions of AIDS-related Kaposi's sarcoma, cutaneous T-cell lymphoma (mycosis fungoides), keratinization disorders (e.g., ichthyosis, Pityriasis rubra pilaris, and Darier's disease, Grover's disease, keratosis pilaris, pitted keratolysis, seborrheic keratosis), palmoplantar hyperkeratosis, xerosis, acanthosis nigricans, plantar hyperkeratosis, carcinoma (e.g., basal cell and squamous cell carcinomas, cutaneous T-cell lymphoma (mycosis fungoides), precancerous actinic keratosis, keratoacanthoma, porokeratosis, and cutaneous sarcoidosis, hidradenitis suppurativa, wrinkles, age related skin changes, abnormal or diminished synthesis of collagen, shallowness, and elastin, diminished levels of collagen and elastin in the dermis, skin atrophy, Anetoderma, stretch marks, skin lines, fine lines, thinning of skin, coarse wrinkles, tactile roughness, skin thickening due to elastosis of photoaging, nodular elastoidosis, loss or reduction of skin resiliency, elasticity and recoilability, lack of skin resiliency, and older looking skin. Such compositions are also used to treat pigmentary skin changes such as freckles, solar lentigo, ephelides, and melasma, as well as corns and callosities.

Compositions Including a Retinoid and/or Arvelexin (with or without a Plant-Derived Biopolymer)

In some embodiments, the dermatological composition includes a retinoid as a first primary agent and arvelexin as a second primary agent. In some embodiments, the dermatological composition includes a retinoid as a first primary agent, arvelexin as a second primary agent, and cutin, lignin or suberin as a third primary agent. In some embodiments, the dermatological composition includes a retinoid as a first primary agent, arvelexin as a second primary agent, cutin, or lignin as a third primary agent, and suberin as a fourth primary agent. In addition to the advantages discussed above with respect to compositions that include a retinoid and cutin, lignin and/or suberin, the compositions may also include arvelexin. Such embodiments may optionally include one or more supplemental agents, and/or a vehicle such as one or more supplemental agents, and/or vehicles discussed in detail above.

Like astaxanthin, arvelexin's antioxidant, anti-inflammatory, and anti-UV properties can potentiate the effects of the retinoid, conferring an additional advantage for such compositions. In addition, arvelexin also has anti-tumor properties, which give an additional layer of protection for subjects experiencing UV sensitivity (e.g., photosensitization) associated with retinoid treatment and provides protection against UV-induced mutation causing precancer growths and skin cancers.

In accordance with the embodiments described herein, compositions that include a retinoid and arvelexin may be used to treat or prevent any one or more of the following conditions: acne (e.g., acne vulgaris, cystic acne, and acne rosacea, acne conglobata), warts, condyloma, flat warts (verruca plana), plantar warts, verruca vulgaris, molluscum contagiosum, bowenoid papulosis, porokeratosis, dyshidrotic eczema (e.g., eczema of the hands and feet), psoriasis, (e.g., ichthyosis, Pityriasis rubra pilaris, and Darier's disease, Grover's disease, keratosis pilaris, pitted keratolysis, seborrheic keratosis, palmoplantar hyperkeratosis, xerosis, acanthosis nigricans, plantar hyperkeratosis, seborrheic dermatitis, atopic dermatitis, aged skin (e.g., photoaged skin or chronologically aged skin), skin cancer, post-inflammatory hyperpigmentation, cutaneous lesions of AIDS-related Kaposi's sarcoma, keratinization disorders (e.g., ichthyosis, Pityriasis rubra pilaris, and Darier's disease, keratosis pilaris, pitting keratolysis), carcinoma (e.g., basal cell and squamous cell carcinomas, cutaneous T-cell lymphoma (mycosis fungoides), precancerous actinic keratosis, keratoacanthoma, porokeratosis, and cutaneous sarcoidosis, hidradenitis suppurativa, wrinkles, age related skin changes, abnormal or diminished synthesis of collagen and elastin, diminished levels of collagen, shallowness, and elastin in the dermis, stretch marks (striae), skin atrophy, skin lines, fine lines, thinning of skin, coarse wrinkles, tactile roughness, skin thickening due to elastosis of photoaging, loss or reduction of skin resiliency, lack of skin resiliency, elasticity and recoilability, and older looking skin. Such compositions also are used to treat pigmentary skin changes such as freckles, solar lentigo, ephelides, and melasma as well as corns and callosities.

Compositions Including Astaxanthin and/or a Plant-Derived Biopolymer

In some embodiments, the dermatological composition includes astaxanthin as a first primary agent and cutin as a second primary agent. In some embodiments, the dermatological composition includes astaxanthin as a first primary agent and suberin as a second primary agent. In some embodiments, the dermatological composition includes astaxanthin as a first primary agent, cutin, lignin as a second primary agent, and suberin as a third primary agent. In some embodiments, the dermatological composition includes astaxanthin as a first primary agent, and one or more of the supplemental agents. Such embodiments may optionally include one or more supplemental agents, and/or a vehicle such as one or more supplemental agents, and/or vehicles discussed in detail above.

On its own, astaxanthin's antioxidant, anti-inflammatory, anti-tumor, and anti-UV properties confers an advantage for many compositions including, but not limited to, sunscreens. When combined with cutin and/or suberin, or lignin, the effects of the astaxanthin are potentiated. For example, in embodiments where the composition is a sunscreen containing astaxanthin, cutin, lignin, and/or suberin, a subject that applies the composition upon exposure to the sun and its UV rays would not only have protection from damage from the sun and other environmental factors, but would also maintain the skin's barrier function, conferring an advantage over using sunscreen alone.

Compositions Including Astaxanthin and/or an Antimicrobial Agent (with or without a Plant-Derived Biopolymer)

In some embodiments, the dermatological composition includes astaxanthin as a first primary agent and lysozyme as a second primary agent. In some embodiments, the dermatological composition includes astaxanthin as a first primary agent and nisin as a second primary agent. In some embodiments, the dermatological composition includes astaxanthin as a first primary agent, lysozyme as a second primary agent, and nisin as a third primary agent. In some embodiments, the dermatological composition includes astaxanthin as a first primary agent, lysozyme as a second primary agent, and cutin, lignin or suberin as a fourth agent. In some embodiments, the dermatological composition includes astaxanthin as a first primary agent, nisin as a second primary agent, and cutin or suberin, or lignin as a fourth agent. In some embodiments, the dermatological composition includes astaxanthin as a first primary agent, lysozyme as a second primary agent, nisin as a third primary agent, and cutin or suberin, or lignin as a fourth agent. Biopolymers (suberin, cutin, lignin) have their own antimicrobial (antiviral, antifungal/yeast, and antibacterial) properties. When combined with lysozyme, the biopolymers (suberin, cutin, lignin) provide a greater stability to lysozyme degradation, which provide a synergistic and additive antimicrobial response. In some embodiments, the dermatological composition includes lysozyme or nisin as a first primary agent, and one or more of the supplemental agents. The embodiments above may optionally include one or more supplemental agents, and/or a vehicle such as one or more supplemental agents, and/or vehicles discussed in detail above.

On its own, astaxanthin has antimicrobial (e.g., antibacterial, antifungal) properties that, in combination with its anti-inflammatory properties, confer advantages when used to prevent or treat microbial infections. When combined with other antibacterial agents like lysozyme and/or nisin, or supplemental agent(s) with antimicrobial properties, the effect is potentiated. Thus, such a combination may be used in compositions that are used in products such as mouthwashes and toothpaste to treat or to prevent gingivitis, oral thrush, oral candidiasis, vagina candidiasis, Candida balanitis, other infectious and inflammatory conditions associated with gums or oropharyngeal mucosa, and anogenital mucosa, or in products to treat or prevent acne vulgaris, impetigo, MRSA (Methicillin-resistant Staphylococcus aureus), folliculitis of skin, hidradenitis suppurativa, or scalp or other hair bearing skin, or other skin ailments, and for acute and chronic wound care, pyoderma gangrenosum, pressure ulcer/skin ulcer/dressing, post-surgical/skin biopsy dressing/wound care, wound care for burn, first degree burn, second degree burn, third degree burn. In one embodiment, such compositions may be used for the treatment, protection, care, cleansing and/or cleaning the skin, lips and/or hair, and/or for making up the skin, lips, eyelashes and/or body, and/or anogenital areas, and/or intranasal area, and/or external ear canal, or ear, and armpits. The above composition/combination with their anti-inflammatory and antimicrobial properties can also be used in deodorants/antiperspirants products to control/prevent microbials growths (i.e. Staphylococcus epidemidis, and Corynebacterium spp.) which are known to contribute to the production of odor in armpits/groin. Additionally, both Lysozyme and Nisin also act as preservatives which may limit (or alleviate) the need to use preservatives which are known to cause dermatitis in deodorant/antiperspirant products. In one aspect, the composition may be a solid, liquid, or semi-liquid preparation, such as a shampoo, conditioner, liquid soap, gel, hydrogel, solution, soap, other soap or cleansing bar, or wipe, or spay, or pellets, sticks, or powder. The composition may also be packaged as an aerosol composition also comprising a propellant under pressure. In another aspect, the composition may be prepared to have an oral use or administration, for example a toothpaste. For oral administration, the composition of the invention may be in any form suitable, particularly in the form of an oral solution, a syrup, a tablet, paste, capsule, or of a food or nutritional supplement, troches, lozenges, pledget. The cleansing agents may be used in a personal care capacity, or may be used in a clinical setting, for example as a topical treatment for preventing infection in a surgical setting, examination room, or other medical, dental and veterinary medical setting, as well as sports and various occupational settings. The cleansing agents may also be used as hand sanitizer, oral sanitizer, rinse, skin sanitizer, disinfectant, antiseptic, anti-microbial. The cleansing agents can be used for all cleansing purposes including for use in pediatric and adult age groups. The cleansing agents can be used for all cleansing purposes in animal/pets. The cleansing agents may also be used in meat handling/packaging and fruit/plant handling, food packaging, as preservatives in food, fruits, dairy products, vegetables.

Compositions Including Astaxanthin and/or an Antineoplastic Agent (with or without a Plant-Derived Biopolymer)

In some embodiments, the dermatological composition includes astaxanthin as a first primary agent and an antineoplastic agent (e.g. 5-Fluorouracil (5-FU), imiquimod, mechloretharmine, a treatment of cutaneous T-cell lymphoma (mycosis fungoides)). In some embodiments, dermatological composition includes astaxanthin as a first primary agent, cutin or lignin as a second primary agent, and an antineoplastic agent (e.g. 5-FU, imiquimod, mechlorethamine). In some embodiments, dermatological composition includes astaxanthin as a first primary agent, lignin as a second primary agent, and an antineoplastic agent (e.g. 5-FU, imiquimod, mechlorethamine). In some embodiments, the dermatological composition includes astaxanthin as a first primary agent, suberin as a second primary agent, and an antineoplastic agent (e.g. 5-FU, imiquimod, mechlorethamine). In some embodiments, the dermatological composition includes astaxanthin as a first primary agent, cutin or lignin as a second primary agent, suberin or lignin as a third primary agent, and an antineoplastic agent (e.g. 5-FU, imiquimod, mechlorethamine). In some embodiments, the dermatological composition includes an antineoplastic agent (e.g. 5-FU, imiquimod, mechlorethamine) as a first primary agent, and one or more of the supplemental agents. Such embodiments may optionally include one or more additional supplemental agents, and/or a vehicle such as one or more supplemental agents, and/or vehicles discussed in detail above.

Common side effects of antineoplastic agents (e.g. 5-Fluorouracil (5-F), imiquimod, and mechlorethamine) include severe skin irritation, erythema, dryness, peeling, burning, edema, blisters, erosion, pain, and sun sensitivity especially on the exposed skin areas which are usually unsightly and ultimately lead to discontinuation of these medications. These side effects can be so severe that compliance becomes a major problem. The anti-inflammatory, antioxidant, UV-blocking effects, wound healing properties and superb humectant capabilities of biopolymers (cutin, suberin, lignin), and astaxanthin (or arvelexin) provide the rationale for combining with the above antineoplastic agents. Additionally, biopolymers, astaxanthin and arvelexin have their own antineoplastic/antitumor/anticancer properties which when combined with antineoplastic agents (5-FU, imiquimod, mechlorethamine) may potentiate their activities and thus may provide additive and synergistic anticancer activities.

Astaxanthin's antitumor, antioxidant, anti-inflammatory, and anti-UV properties confer an additional advantage for treating malignant or benign tumors on the skin, or precancerous (e.g., in actinic keratosis), porokeratosis. Such combination may also be used in the treatment of condyloma, flat warts (verruca plana), plantar warts, molluscum contagiosum, verruca vulgaris, bowenoid papulosis. Thus, in some embodiments, astaxanthin can be combined with an antineoplastic agent such as 5-FU or imiquimod, mechlorethamine in a composition to treat such skin lesions or growths. When combined with cutin, lignin and/or suberin, the composition may provide additional advantages by maintaining and/or improving the barrier function of the skin during treatment.

In some embodiments, astaxanthin can be combined with both antineoplastic agents 5-Fluorouracil (5-FU) and Imiquimod. In some embodiments, astaxanthin can be added to the combination of antineoplastic agents 5-FU and Imiquimod and a biopolymer such as suberin, or cutin, or lignan. Such embodiments may optionally include one or more supplemental agents, and/or a vehicle such as one or more supplemental agents, and/or vehicles discussed in detail above.

In some embodiments, astaxanthin can be combined with both antineoplastic agents 5-FU, and Imiquimod. Such combination can be used for treating malignant or benign and precancerous tumors on the skin (e.g., in actinic keratosis), porokeratosis as well as for treatment of condyloma, flat warts (verruca plana), plantar warts, verruca vulgaris, molluscum contagiosum, bowenoid papulosis. Such embodiments may optionally include one or more supplemental agents, and/or a vehicle such as one or more supplemental agents, and/or vehicles discussed in detail above.

In some embodiments, astaxanthin can be combined with mechlorethamine. Mechlorethamine (e.g., Gel: 0.016% w/w of mechlorethamine, equivalent to 0.02% mechlorethamine HCl) is used for treatment of cutaneous T-cell lymphoma (mycosis fungoides). The most common side effects of mechlorethamine, include severe dermatitis, inflammatory reaction, causing redness, swelling, itching, skin ulceration or blisters, bacterial skin infection, and darkening of areas of skin (hyperpigmentation). These side effects at times are so severe that the treatment will often have to be discontinued. Due to its antioxidant, anti-inflammatory and antimicrobial and wound healing properties, when astaxanthin (and/or arvelexin as discussed below) is combined with a plant-derived biopolymer (e.g., suberin, cutin, lignin), the side effects of mechlorethamine will be significantly reduced, making the application far more tolerable. Furthermore, anti-tumor, anti-neoplastic activities of astaxanthin (and/or arvelexin as discussed below), when combined with plant-derived biopolymers will potentiate the anti-neoplastic activity of mechlorethamine against cutaneous T-cell lymphoma (mycosis fungoidis).

Compositions Including Arvelexin and/or a Plant-Derived Biopolymer

In some embodiments, the dermatological composition includes arvelexin as a first primary agent and cutin as a second primary agent. In some embodiments, the dermatological composition includes arvelexin as a first primary agent and suberin as a second primary agent. In some embodiments, the dermatological composition includes arvelexin as a first primary agent and lignin as a second primary agent. In some embodiments, the dermatological composition includes arvelexin as a first primary agent, cutin or lignin as a second primary agent, and suberin or lignin as a third primary agent. Such embodiments may optionally include one or more supplemental agents, and/or a vehicle such as one or more supplemental agents, and/or vehicles discussed in detail above.

On its own, Arvelexin's antioxidant, anti-inflammatory, anti-tumor, and anti-UV properties confers an advantage for many compositions including, but not limited to, sunscreens. When combined with cutin, lignin and/or suberin, the effects of the arvelexin are potentiated. For example, in embodiments where the composition is a sunscreen containing arvelexin, cutin, lignin and/or suberin, a subject that applies the composition upon exposure to the sun and its UV rays would not only have protection from damage from the sun and other environmental facts, but would also maintain the skin's barrier function, conferring an advantage over using sunscreen alone.

Compositions Including Arvelexin and/or an Antimicrobial Agent (with or without a Plant-Derived Biopolymer)

In some embodiments, the dermatological composition includes arvelexin as a first primary agent and lysozyme as a second primary agent. In some embodiments, the dermatological composition includes arvelexin as a first primary agent and nisin as a second primary agent. In some embodiments, the dermatological composition includes arvelexin as a first primary agent, lysozyme as a second primary agent, and nisin as a third primary agent. In some embodiments, the dermatological composition includes arvelexin as a first primary agent, lysozyme as a second primary agent, and cutin or lignin, or suberin as a fourth agent. In some embodiments, the dermatological composition includes arvelexin as a first primary agent, nisin as a second primary agent, and cutin, lignin or suberin as a fourth agent. In some embodiments, the dermatological composition includes arvelexin as a first primary agent, lysozyme as a second primary agent, nisin as a third primary agent, and cutin, lignin or suberin as a fourth agent. Such embodiments may optionally include one or more supplemental agents, and/or a vehicle such as one or more supplemental agents, and/or vehicles discussed in detail above.

On its own, arvelexin has antimicrobial (e.g., antibacterial/antifungal) properties that, in combination with its anti-inflammatory properties, confer advantages when used to prevent or treat microbial infections. When combined with other antibacterial agents like lysozyme and/or nisin, and/or supplemental agents with antimicrobial properties, the effect is potentiated. Thus, such a combination may be used in compositions that are used in products such as mouthwashes and toothpaste to treat or to prevent gingivitis, oral thrush, oral candidiasis, Candida balanitis, vagina candidiasis, other infectious and inflammatory conditions associated with gums or oropharyngeal mucosa, aphthous stomatitis, or in products to treat or prevent acne vulgaris, impetigo, hidradenitis suppurativa, MRSA (Methicillin-resistant Staphylococcus aureus), folliculitis of skin or scalp or other hair bearing skin, or other skin ailments, and for acute and chronic wound care, pyoderma gangrenosum, pressure ulcer/skin ulcer/dressing, post-surgical/skin biopsy dressing/wound care. Wound care for burn, first degree burn, second degree burn, third degree burn, lubricants. In one embodiment, such compositions may be used for the treatment, protection, care, cleansing and/or cleaning the skin, lips and/or hair, and/or for making up the skin, lips, eyelashes and/or body, and/or anogenital areas, and/or intranasal area, and/or external ear canal or ears, and armpits. The above composition/combination with their anti-inflammatory and antimicrobial properties can also be used in deodorants/antiperspirants products to control/prevent microbial growths (i.e. Staphylococcus epidemidis, and Corynebacterium spp.) which are known to contribute in production of odor in armpits/groin. Additionally, both Lysozyme and Nisin also act as preservatives which may limit (or alleviate) the need to use preservatives which are known to cause dermatitis in deodorant/antiperspirant products. In one aspect, the composition may be a solid, liquid, or semi-liquid preparation, such as a shampoo, conditioner, liquid soap, gel hydrogel, solution, soap, other soap or cleansing bar. The composition may also be packaged as an aerosol composition also comprising a propellant under pressure. In another aspect, the composition may be prepared to have an oral use or administration, for example a toothpaste. For oral administration, the composition of the invention may be in any form suitable, particularly in the form of an oral solution, a syrup, a tablet, paste, capsule, or of a food or nutritional supplement, troches, lozenges, pledget, chewing gum. The cleansing agents may be used in a personal care capacity, or may be used in a clinical setting, for example as a topical treatment for preventing infection in a surgical setting, examination room, or other medical, dental and veterinary medical setting, spa, sports and various occupational settings. The cleansing agents may also be used as hand sanitizer, rinse, oral sanitizer, skin sanitizer, disinfectant, antiseptic, anti-microbial. The cleansing agents can be used for all cleansing purposes including for use in pediatric and adult age groups. The cleansing agents can be used for all cleansing purposes in animal/pets. The cleansing agents may also be used in meat handling/packaging, and fruit/plant handling, food packaging, as preservatives in food, fruits, dairy products, vegetables.

Compositions Including Arvelexin and/or an Antineoplastic Agent (with or without a Plant-Derived Biopolymer)

In some embodiments, the dermatological composition includes arvelexin as a first primary agent and an antineoplastic agent (e.g. 5-Fluorouracil (5-FU), imiquimod, mcchlorethamine). In some embodiments, dermatological composition includes arvelexin as a first primary agent, cutin as a second primary agent, and an antineoplastic agent (e.g. 5-FU, imiquimod). In some embodiments, the dermatological composition includes arvelexin as a first primary agent, suberin as a second primary agent, and an antineoplastic agent (e.g. 5-Flourouracil (5-FU), imiquimod, mechliorethamine). In some embodiments, dermatological composition includes arvelexin as a first primary agent, lignin as a second primary agent, and an antineoplastic agent (e.g. 5-FU, imiquimod). In some embodiments, the dermatological composition includes arvelexin as a first primary agent, cutin or lignin as a second primary agent, suberin or lignin as a third primary agent, and an antineoplastic agent (e.g. 5-FU, imiquimod, mechlorethamine). Such embodiments may optionally include one or more additional supplemental agents, and/or a vehicle such as one or more supplemental agents, and/or vehicles discussed in detail above.

Arvelexin's antitumor, antioxidant, anti-inflammatory, and anti-UV properties confer an additional advantage for treating malignant or benign and precancerous tumors on the skin (e.g., in actinic keratosis), porokeratosis, as well as for treatment of condyloma, flat warts (verruca plana), plantar warts, verruca vulgaris, molluscum contagiosum, bowenoid papulosis. Thus, in some embodiments, arvelexin can be combined with an antineoplastic agent such as 5-FU or imiquimod in a composition to treat such skin growths or lesions. When combined with cutin, lignin and/or suberin, the composition may provide additional advantages by maintaining and/or improving the barrier function of the skin during treatment. Additionally, the anti-tumor/anti-neoplastic activity of arvelexin may potentiate the anti-tumor activity of 5-FU, imiquimod, and mechlorethamine and may lead to a synergistic effect.

In some embodiments, arvelexin can be combined with both antineoplastic agents 5-FU and Imiquimod. Such combination can be used for treating malignant or benign and precancerous tumors on the skin (e.g., in actinic keratosis), porokeratosis as well as for treatment of condyloma, flat warts (verruca plana), plantar warts, verruca vulgaris, molluscum contagiosum, bowenoid papulosis. Such embodiments may optionally include one or more supplemental agents, and/or a vehicle such as one or more supplemental agents, and/or vehicles discussed in detail above.

In some embodiments, arvelexin can be combined with mechlorethamine. Mechlorethamine (e.g., Gel: 0.016% w/w of mechlorethamine, equivalent to 0.02% mechlorethamine HCl) is used for treatment of cutaneous T-cell lymphoma (mycosis fungoides). The most common side effects of mechlorethamine, include severe dermatitis, inflammatory reaction, causing redness, swelling, itching, skin ulceration or blisters, bacterial skin infection, and darkening of areas of skin (hyperpigmentation). These side effects at times are so severe that the treatment will often have to be discontinued. Due to its antioxidant, anti-inflammatory and antimicrobial properties, when arvelexin (and/or astaxanthin as discussed above) is combined with a plant-derived biopolymer (e.g., suberin, cutin, lignin, the side effects of mechlorethamine will be significantly reduced, making the application far more tolerable. Furthermore, anti-tumor, anti-neoplastic activities of arvelexin (and/or astaxanthin as discussed above), when combined with plant-derived biopolymers will potentiate the anti-neoplastic activity of mechlorethamine against cutaneous T-cell lymphoma (mycosis fungoidis).

Compositions Including Lysozyme and/or Nisin

In some embodiments, the dermatological composition includes a lysozyme as a first primary agent and nisin as a second primary agent. In some embodiments, the dermatological composition includes a lysozyme as a first primary agent and nisin as a second primary agent. In some embodiments, the dermatological composition includes lysozyme or nisin as a first primary agent, and one or more supplemental agents. Such embodiments may optionally include one or more supplemental agents, and/or a vehicle such as one or more supplemental agents, and/or vehicles discussed in detail above.

A combination of lysozyme and/or nisin may be used in compositions that are used in products such as mouthwashes and toothpaste to prevent gingivitis, oral thrush, oral candidiasis, vaginal candidiasis, Candida balanitis or in products to treat or prevent acne vulgaris, folliculitis impetigo, hidradenitis suppurativa, MRSA infection (Methicillin resistant staph aureus), or other skin ailments. In one embodiment, such compositions may be used for the treatment, protection, care, cleansing and/or cleaning the skin, lips and/or hair, and/or for making up the skin, lips, eyelashes and/or body. In one aspect, the composition may be a solid, liquid, or semi-liquid preparation, such as a shampoo, conditioner, liquid soap, gel soap, other soap or cleansing bar. The composition may also be packaged as an aerosol composition also comprising a propellant under pressure. In another aspect, the composition may be prepared to have an oral use or administration, for example a toothpaste. For oral administration, the composition of the invention may be in any form suitable, particularly in the form of an oral solution, a syrup, a tablet, paste, capsule, or of a food or nutritional supplement, troches, lozenges, pledget, chewing gum. The cleansing agents may be used in a personal care capacity, or may be used in a clinical setting, for example as a topical treatment for preventing infection in a surgical, examination, or other medical setting. In another embodiment a preservative (e.g., parabens or other commonly used preservatives in dermatological compositions) may be replaced by nisin or a related lantibiotic to reduce sensitivity to preservatives in certain subjects.

Methods of Treating or Preventing Dermatological Conditions

The compositions described in the embodiments above may be used in methods for treating or preventing infection and dermatological conditions. Such methods may include administering one or more dermatological compositions (e.g., those disclosed in the embodiments above) to a subject. In some embodiments, a dermatological composition is administered to a subject to treat or prevent a dermatological condition.

The dermatological composition used in some embodiments may include one or more primary agents. In some embodiments, the one or more primary agents may be selected from a retinoid, astaxanthin, arvelexin, a plant-derived biopolymer or extracts thereof (e.g. cutin and/or suberin, and/or lignin), lysozyme, and nisin. In certain such embodiments, the one or more primary agents include a first primary agent and, optionally, one or more supplemental agents. In some embodiments, the methods for treating or preventing a dermatological condition include administering two or more dermatological compositions to a subject. In such examples, the method may include administering a first dermatological composition in conjunction with a second dermatological composition. In such embodiments, the first and second compositions may both be administered topically or orally; or the first and second compositions may be administered orally and topically, respectively. In certain embodiments, the first and second compositions may be administered at the same time or approximately the same time or may be temporally administered before or after each other. In some embodiments, the first and second compositions are sold together as part of a kit or may be sold separately. Examples of primary and supplemental agents that may be used in the compositions suitable for use in the methods described herein are discussed in detail above.

The dermatological composition used in some embodiments may include two or more primary agents. In some embodiments, the two or more primary agents may be selected from a retinoid, astaxanthin, arvelexin, a plant-derived biopolymer or extracts thereof (e.g. cutin and/or suberin, or lignin), lysozyme, and nisin. In certain such embodiments, the two or more primary agents include a first primary agent, a second primary agent, and, optionally, one or more supplemental agents. Examples of primary and supplemental agents that may be used in the compositions suitable for use in the methods described herein are discussed in detail above.

The dermatological composition used in some embodiments may include three or more primary agents. In some embodiments, the three or more primary agents may be selected from a retinoid, astaxanthin, arvelexin, a plant-derived biopolymer or extracts thereof (e.g. cutin, lignin and/or suberin), lysozyme, and nisin. In certain such embodiments, the three or more primary agents include a first primary agent, a second primary agent, a third primary agent, and, optionally, one or more supplemental agents. Examples of primary and supplemental agents that may be used in the compositions suitable for use in the methods described herein are discussed in detail above.

According to some of the embodiments described herein an effective amount of the dermatological compositions (inclusive of the agents contained therein) is administered to the subject. An effective amount is an amount affective at dosages and for periods of time sufficient to achieve a desired result. The composition according to any of the embodiments above may include a concentration or dose of each primary or supplemental agent sufficient to achieve a desired result in accordance with the embodiments described above. Specific non-limiting examples of concentrations and formulations are disclosed throughout the disclosure.

The compositions described herein may be ingested, injected, or applied to the skin (on any skin area of the body), scalp, hair, nails or mucous membranes according to some embodiments. According to some of the embodiments described herein, the dermatological compositions are administered to the subject by mucosal or cutaneous topical administration in the form of a lotion, gel, hydrogel, wipe, ointment, cream, foam, liquid solution, or paste, spray, sticks, mask to the skin (e.g., to the face, arms, legs, ears, neck, trunk, body), scalp, nails, gums, genitals, eyes, intravaginally, hair, or mouth, intranasal, or inside ear canal. Alternatively, the dermatological composition may be administered orally as a capsule, gel capsule, tablet, or liquid solution, used by insertion applicator. Examples of vehicles that may be used in the dermatological compositions to produce the form of the product are discussed above. Dermatological products in which composition or compositions may be used in are described in detail above.

In some embodiments, the subject may be any animal (e.g., mammal) including, but not limited to, humans, non-human primates, rodents, dogs, cats, horses, and other domesticated animals to which the compositions may be administered.

The compositions described herein may be used to treat or prevent a dermatological condition such as a disease, infection or condition. Non-limiting examples of the diseases, infections, and/or conditions that may be treated or prevented by the methods according to the embodiments described herein include (i) treatment or prevention of the aging process (i.e., reversal of photo-aging and/or chronological aging) such as reducing (improving) or preventing wrinkles, freckles, and fine lines, coarse wrinkles, tactile roughness, shallowness, skin atrophy, stretch marks (striae), acanthosis nigricans, improving elasticity, nodular elastodosis, and/or tone of the skin, improving dull skin, treating internal damage to the skin caused by exposure to UV radiation, improving firmness, reducing weathered appearance, and smoothing of skin; (ii) treatment or prevention of dry skin by removal of skin cells and/or improving hydration and/or improving skin barrier function by reducing TEWL; (iii) treatment or prevention of skin damage caused by environmental conditions (e.g., pollution, hard water, surfactants, preservatives, perfumes, sun, wind, high temperatures); (iv) treatment of pathologic dermatologic conditions or diseases such as acne, keratosis pilaris, pitted keratolysis, palmo-plantar hyperkeratosis, xerosis, plantar hyperkeratosis, seborrheic keratosis, Pityriasis rubra pilaris, and Darier's disease, Grover's disease, pitted keratolysis, seborrheic keratosis, seborrheic dermatitis, atopic dermatitis, callosities, corn, psoriasis vulgaris, diaper dermatitis, various forms of ichthyosis, xerosis, acanthosis nigricans, warts, verruca plana, flat warts, condyloma, verruca vulgaris, molluscum contagiosum, bowenoid papulosis, porokeratosis, actinic keratosis; (v) treatment or prevention of microbial infection or conditions caused by microbes or microbial infections (e.g., folliculitis, gingivitis), or (vi) a combination of the conditions disclosed in (i)-(v). Non-limiting specific examples of compositions and their use in treating or preventing particular conditions are disclosed throughout the disclosure.

In other embodiments, compositions containing plant-derived biopolymers (suberin, cutin, lignin), astaxanthin, and/or arvelexin may be used to treat wounds due to their wound healing properties. Examples of wounds that may be treated using a plant-derived biopolymers (suberin, cutin, lignin), astaxanthin, and/or arvelexin include, but are not limited to, an acute and/or chronic ulcer, a wound from surgery, a wound from biopsy, a wound from burn (first degree, second degree and third degree skin burn), a pressure ulcer, chronic non-healing ulcers including but not limited to pyogenic granuloma, stasis ulcer, diabetic foot ulcer, Behcet's disease, aphthous stomatitis, discoid lupus, lupus ulcer, various granulomatous skin/mucosal diseases such as necrobiosis lipoidica diabeticorum, and other wounds suitable for treatment.

As described in detail above, in certain embodiments, the compositions described herein may contain a cosmetically acceptable vehicle or carrier, i.e., compatible with the skin, mucous membranes, lips, nails, or hair. For example, the compositions described herein may be in the form of an aqueous solution, hydro-alcoholic or oil solution; an oil-in-water emulsion, water-in-oil or multiple emulsions; they also may be in the form of creams, suspensions, or powders, suitable for application to the skin, mucous membranes, lips, nails, and/or hair. The compositions described herein may have the appearance of a cream, a lotion, solution, a milk, a serum, an ointment, a gel, a hydrogel, a wipe, paste or a foam. Alternatively, the compositions described herein may be in solid form, such as a stick or be applied to the skin in aerosol form. They can be used as a skin care product and/or as a skin makeup product. For example, the compositions may be applied as a protective product and/or care of the skin, or as an anti-wrinkle composition and/or anti-aging, especially facial skin and/or neck, hands, forearms. In particular, the compositions find application as a composition to have a tightening effect on the skin. One may also consider an application in the field of cosmetic compositions of the facial skin and body, such as lipsticks, the foundation, tinted creams, concealer sticks, or sunscreen compositions or artificial tanning. Additional products are disclosed in detail above. In other examples, the compositions described herein may be used in hair products, troches, lozenges, pledget, chewing gum.

According to another aspect, the present invention relates to a cosmetic treatment method for treating aged skin and/or combating of curative and/or preventive the skin aging phenomena manner of applying, on the surface of the skin, an amount effective amount of a composition as defined above. In other words, a composition containing suberin, retinol, or other skin conditioning agent may be used to obtain the desired action. The present invention also relates to a cosmetic treatment process for the skin in order to obtain a tensioning effect, that is to say a mechanical and direct smoothing of the surface of the skin, consisting in applying on the skin, effective amount of a composition as defined above. The present invention relates, in the same manner, a cosmetic treatment process to protect the skin and/or hair against all types of external and environmental effects. The treatment methods of the embodiments described above can be administered by applying the cosmetic compositions as defined above according to typical techniques for using these compositions, for example: application of creams, gels, hydrogel, ointment, foam, sera, lotions, milks, shampoos or other compositions on the skin or hair, or, toothpaste applied to the gums, oral mucosa, tongue.

In some embodiments, compositions including astaxanthin and/or arvelexin in combination with one or more of (i) lysozyme, (ii) nisin, and/or (iii) plant-derived biopolymers (suberin, cutin, or lignin) can be used in the form of face wash, face cleanser, aftershave, or hair cleanser (e.g., lotion, foam, gel, hydrogel, foam, liquid, bar, deodorants, antiperspirants, shampoo, wipe, etc.) to provide treatment of individuals with sensitive skin such as infants or children (pediatrics); adults whose skin burns easily or tans easily; people with an extremely fair complexion; patients with atopic dermatitis, eczema, dyshidrotic eczema, seborrheic dermatitis, hyperpigmentation, freckles, solar lentigo, ephelides, melasma, vitiligo, piebaldism folliculitis, impetigo, MRSA (Methicillin-resistant Staphylococcus aureus), various forms of skin infection, acne vulgaris, or hidradenitis suppurativa; albinos; xeroderma pigmentosa, or burn patients.

Similarly, sunscreens containing astaxanthin, arvelexin, supplemental agents (e.g., Pterostilbene, Equol (4′,7-isoflavandiol) (R-equol, racemic equol or S-equol), Quercetin), either alone or in combination with a plant-derived biopolymer (suberin, cutin, lignin) due to their superior antioxidant, anti-microbial, and anti-inflammatory properties and the fact that they possess their own UV light blocking properties, will provide advantageous over current sunscreens for individuals with sensitive skin such in the pediatric or adult population; individuals whose skin burns easily or tans easily; people with an extremely fair complexion; patients with atopic dermatitis, eczema, seborrheic dermatitis, hyperpigmentation, freckles, ephelides, melasma, vitiligo, piebaldism; burn patients, albinos; xeroderma pigmentosa, patients with a history of skin cancer and/or precancerous actinic keratosis; or individuals with scalp hair loss and/or androgenic alopecia.

From the foregoing, it will be appreciated that specific embodiments of the invention have been described herein for purposes of illustration, but that various modifications may be made without deviating from the scope of the invention.

### WORKING EXAMPLES

The following examples are intended to illustrate various embodiments of the invention. As such, the specific embodiments discussed are not to be construed as limitations on the scope of the invention.

The following examples include non-limiting formulations covered by the embodiments discussed above. The formulations may include one or more primary and/or supplemental agent ingredients, each having a range of possible concentrations. In such formulations, any combination of ingredients falling within that range is contemplated as an embodiment.

The examples may also include formulations that include one or more ingredients, each having one or more possible specified concentrations. In such formulations, every combination of ingredients and concentrations contemplated by each formulation corresponds to a separate embodiment. Non-limiting exemplary formulations may include ingredients with corresponding concentrations as follows:

| Formulation No. | Ingredients (% w/w or w/v)* |
|---|---|
| F₁ | Ingredient 1 (X₁ %, X₂ %, X₃ %) |
| F₂ | Ingredient 1 (X₁ %) |
| | Ingredient 2 (Y₁ %, Y₂ %, Y₃ %) |
| F₃ | Ingredient 1 (X₁ %) |
| | Ingredient 2 (Y₁ %, Y₂ %, Y₃ %) |
| | Ingredient 3 (Z₁ %, Z₂ %, Z₃ %) |

*unless specified otherwise

Accordingly, non-limiting exemplary Formula F₁ includes three embodiments corresponding to a first composition that includes Ingredient 1 at a concentration of X₁%, a second composition that includes Ingredient 1 at a concentration of X₂%, and a third composition that includes Ingredient 1 at a concentration of X₃%.

Non-limiting exemplary Formula F₂ includes three embodiments corresponding to a first composition that includes Ingredient 1 at a concentration of X₁% and Ingredient 2 at a concentration of Y₁%, a second composition that includes Ingredient 1 at a concentration of X₁% and Ingredient 2 at a concentration of Y₂%, and a third composition that includes Ingredient 1 at a concentration of X₁% and Ingredient 2 at a concentration of Y₃%.

Non-limiting exemplary Formula F₃ includes nine embodiments corresponding to a first composition that includes Ingredient 1 at a concentration of X₁%, Ingredient 2 at a concentration of Y₁%, and Ingredient 3 at a concentration of Z₁%; a second composition that includes Ingredient 1 at a concentration of X₁%, Ingredient 2 at a concentration of Y₁%, and Ingredient 3 at a concentration of Z₂%; a third composition that includes Ingredient 1 at a concentration of X₁%, Ingredient 2 at a concentration of Y₁%, and Ingredient 3 at a concentration of Z₃%; a fourth composition that includes Ingredient 1 at a concentration of X₁%, Ingredient 2 at a concentration of Y₂%, and Ingredient 3 at a concentration of Z₁%; a fifth composition that includes Ingredient 1 at a concentration of X₁%, Ingredient 2 at a concentration of Y₂%, and Ingredient 3 at a concentration of Z₂%; a sixth composition that includes Ingredient 1 at a concentration of X₁%, Ingredient 2 at a concentration of Y₂%, and Ingredient 3 at a concentration of Z₃%; a seventh composition that includes Ingredient 1 at a concentration of X₁%, Ingredient 2 at a concentration of Y₃%, and Ingredient 3 at a concentration of Z₁%; an eighth composition that includes Ingredient 1 at a concentration of X₁%, Ingredient 2 at a concentration of Y₃%, and Ingredient 3 at a concentration of Z₂%; and a ninth composition that includes Ingredient 1 at a concentration of X₁%, Ingredient 2 at a concentration of Y₃%, and Ingredient 3 at a concentration of Z₃%.

It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Plant-Derived Biopolymer-Based Compositions with or without Arvelexin or Astaxanthin

The rationale for using the plant-derived biopolymers with or without arvelexin or astaxanthin is due to high hydrophilic properties of these compounds, which helps to retain moisture at the site of use. Compositions that include one or more of suberin, cutin, and lignin alone or in combination with arvelexin and/or astaxanthin, and/or in combination with one or more supplemental agents, may be used in many different dermatologic products, as discussed herein. Specific examples of such products include moisturizer compositions (as discussed below). Additional examples can also be found in the working examples below.

Additionally, many skin diseases are of inflammatory nature. Clinically, these diseases usually are dry, peeling, flaky (scaly), and are itchy which makes them prone to developing a secondary skin infection. The anti-inflammatory and antimicrobial properties of these combination products (astaxanthin, arvelexin, or plant-derived biopolymers such as suberin, cutin, or lignin) will provide highly efficacious compositions and will likely be advantageous over all currently available moisturizers without those ingredients.

Table 1 below includes formulations for one or more primary agents used in a plant-derived biopolymer-based composition (with or without arvelexin or astaxanthin). The compositions include at least one plant-derived biopolymer alone or in combination with arvelexin or astaxanthin. Additional active and/or non-active primary or supplemental agents may also be included in or used in conjunction with the formulations below in accordance with the disclosure above. The formulations may also include one or more vehicle ingredients to provide a suitable vehicle for topical administration including, but not limited to, those described above. The compositions may be processed and manufactured according to conventional methods known in the art.

TABLE 1
Formulations for plant-derived biopolymer-based composition (with or without arvelexin
or astaxanthin). The formulations below may be used in conjunction with or further
include one or more of the additional supplemental agents disclosed above.

| Formulation No. | Ingredients (% w/w or w/v, or as indicated) |
|---|---|
| 1 | Suberin (3%) |
| 2 | Cutin (1%-5%) |
| 3 | Cutin (1%) |
| 4 | Cutin (2%) |
| 5 | Cutin (3%) |
| 6 | Cutin (4%) |
| 7 | Cutin (5%) |
| 8 | Suberin (3%) |
| | Astaxanthin (1%-10%) |
| 9 | Suberin (3%) |
| | Astaxanthin (1%) |
| 10 | Suberin (3%) |
| | Astaxanthin (2%) |
| 11 | Suberin (3%) |
| | Astaxanthin (3%) |
| 12 | Suberin (3%) |
| | Astaxanthin (4%) |
| 13 | Suberin (3%) |
| | Astaxanthin (5%) |
| 14 | Suberin (3%) |
| | Astaxanthin (7.5%) |
| 15 | Suberin (3%) |
| | Astaxanthin (10%) |
| 16 | Cutin (1%-5%) |
| | Astaxanthin (1%-10%) |
| 17 | Cutin (1%, 2%, 3%, 4%, or 5%) |
| | Astaxanthin (1%, 2%, 3%, 4%, 5%, 7.5%, or 10%) |
| 18 | Cutin (1%) |
| | Astaxanthin (1%, 2%, 3%, 4%, 5%, 7.5%, or 10%) |
| 19 | Cutin (2%) |
| | Astaxanthin (1%, 2%, 3%, 4%, 5%, 7.5%, or 10%) |
| 20 | Cutin (3%) |
| | Astaxanthin (1%, 2%, 3%, 4%, 5%, 7.5%, or 10%) |
| 21 | Cutin (4%) |
| | Astaxanthin (1%, 2%, 3%, 4%, 5%, 7.5%, or 10%) |
| 22 | Cutin (5%) |
| | Astaxanthin (1%, 2%, 3%, 4%, 5%, 7.5%, or 10%) |
| 23 | Cutin (1%, 2%, 3%, 4%, or 5%) |
| | Astaxanthin (1%) |
| 24 | Cutin (1%, 2%, 3%, 4%, or 5%) |
| | Astaxanthin (2%) |
| 25 | Cutin (1%, 2%, 3%, 4%, or 5%) |
| | Astaxanthin (3%) |
| 26 | Cutin (1%, 2%, 3%, 4%, or 5%) |
| | Astaxanthin (4%) |
| 27 | Cutin (1%, 2%, 3%, 4%, or 5%) |
| | Astaxanthin (5%) |
| 28 | Cutin (1%, 2%, 3%, 4%, or 5%) |
| | Astaxanthin (7.5%) |
| 29 | Cutin (1%, 2%, 3%, 4%, or 5%) |
| | Astaxanthin (10%) |
| 30 | Suberin (3%) |
| | Arvelexin (10 μM-200 μM) |
| 31 | Suberin (3%) |
| | Arvelexin (10 μM, 25 μM, 50 μM, 75 μM, 100 μM, 150 μM, or 200 μM) |
| 32 | Suberin (3%) |
| | Arvelexin (10 μM) |
| 33 | Suberin (3%) |
| | Arvelexin (25 μM) |
| 34 | Suberin (3%) |
| | Arvelexin (50 μM) |
| 35 | Suberin (3%) |
| | Arvelexin (75 μM) |
| 36 | Suberin (3%) |
| | Arvelexin (100 μM) |
| 37 | Suberin (3%) |
| | Arvelexin (150 μM) |
| 38 | Suberin (3%) |
| | Arvelexin (200 μM) |
| 39 | Cutin (1%-5%) |
| | Arvelexin (10 μM-200 μM) |
| 40 | Cutin (1%, 2%, 3%, 4%, or 5%) |
| | Arvelexin (10 μM, 25 μM, 50 μM, 75 μM, 100 μM, 150 μM, or 200 μM) |
| 41 | Cutin (1%) |
| | Arvelexin (10 μM, 25 μM, 50 μM, 75 μM, 100 μM, 150 μM, or 200 μM) |
| 42 | Cutin (2%) |
| | Arvelexin (10 μM, 25 μM, 50 μM, 75 μM, 100 μM, 150 μM, or 200 μM) |
| 43 | Cutin (3%) |
| | Arvelexin (10 μM, 25 μM, 50 μM, 75 μM, 100 μM, 150 μM, or 200 μM) |
| 44 | Cutin (4%) |
| | Arvelexin (10 μM, 25 μM, 50 μM, 75 μM, 100 μM, 150 μM, or 200 μM) |
| 45 | Cutin (5%) |
| | Arvelexin (10 μM, 25 μM, 50 μM, 75 μM, 100 μM, 150 μM, or 200 μM) |
| 46 | Cutin (1%, 2%, 3%, 4%, or 5%) |
| | Arvelexin (10 μM) |
| 47 | Cutin (1%, 2%, 3%, 4%, or 5%) |
| | Arvelexin (25 μM) |
| 48 | Cutin (1%, 2%, 3%, 4%, or 5%) |
| | Arvelexin (50 μM) |
| 49 | Cutin (1%, 2%, 3%, 4%, or 5%) |
| | Arvelexin (75 μM) |
| 50 | Cutin (1%, 2%, 3%, 4%, or 5%) |
| | Arvelexin (100 μM) |
| 51 | Cutin (1%, 2%, 3%, 4%, or 5%) |
| | Arvelexin (150 μM) |
| 52 | Cutin (1%, 2%, 3%, 4%, or 5%) |
| | Arvelexin (200 μM) |
| 53 | Lignin (1%-30%) |
| 54 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| 55 | Lignin (1%-30%) |
| | Astaxanthin (1%-10%) |
| 56 | Lignin (1%-30%) |
| | Astaxanthin (1%, 2%, 3%, 4%, 5%, 7.5%, or 10%) |
| 57 | Lignin (1%-30%) |
| | Astaxanthin (1%) |
| 58 | Lignin (1%-30%) |
| | Astaxanthin (2%) |
| 59 | Lignin (1%-30%) |
| | Astaxanthin (3%) |
| 60 | Lignin (1%-30%) |
| | Astaxanthin (4%) |
| 61 | Lignin (1%-30%) |
| | Astaxanthin (5%) |
| 62 | Lignin (1%-30%) |
| | Astaxanthin (7.5%) |
| 63 | Lignin (1%-30%) |
| | Astaxanthin (10%) |
| 64 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (1%-10%) |
| 65 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| 66 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (1%) |
| 67 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (2%) |
| 68 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (3%) |
| 69 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (4%) |
| 70 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (5%) |
| 71 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (7.5%) |
| 72 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Astaxanthin (10%) |
| 73 | Lignin (1%-30%) |
| | Arvelexin (10 μM-200 μM) |
| 74 | Lignin (1%-30%) |
| | Arvelexin (10 μM, 25 μM, 50 μM, 75 μM, 100 μM, 150 μM, or 200 μM) |
| 75 | Lignin (1%-30%) |
| | Arvelexin (10 μM) |
| 76 | Lignin (1%-30%) |
| | Arvelexin (25 μM) |
| 77 | Lignin (1%-30%) |
| | Arvelexin (50 μM) |
| 78 | Lignin (1%-30%) |
| | Arvelexin (75 μM) |
| 79 | Lignin (1%-30%) |
| | Arvelexin (100 μM) |
| 80 | Lignin (1%-30%) |
| | Arvelexin (150 μM) |
| 81 | Lignin (1%-30%) |
| | Arvelexin (200 μM) |
| 82 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (10 μM-200 μM) |
| 83 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (10 μM, 25 μM, 50 μM, 75 μM, 100 μM, 150 μM, or 200 μM) |
| 84 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (10 μM) |
| 85 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (25 μM) |
| 86 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (50 μM) |
| 87 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (75 μM) |
| 88 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (100 μM) |
| 89 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (150 μM) |
| 90 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, 25%, |
| | 26%, 27%, 28%, 29%, or 30%) |
| | Arvelexin (200 μM) |

Moisturizer Compositions

In one example, the compositions in Table 1 includes formulations for a topical moisturizer composition. The moisturizer compositions include at least one plant-derived biopolymer alone or in combination with arvelexin or astaxanthin. Additional active and/or non-active primary or supplemental agents may also be included in the formulations below in accordance with the disclosure above. The formulations may also include one or more vehicle ingredients to provide a suitable vehicle for topical administration including, but not limited to, those described above. The compositions may be processed and manufactured according to conventional methods known in the art.

The use of moisturizers is commonly recommended as an adjunct treatment for dry, scaly skin conditions such as hyperkeratosis, and various inflammatory skin conditions such as psoriasis vulgaris, atopic dermatitis, diaper dermatitis, allergic dermatitis, retinoid dermatitis, seborrheic dermatitis, poison oak/poison ivy, dry skin/xerosis, asteatotic eczema, dyshidrotic eczema, asteatosis cutis, pemphigus, pemphigus vulgaris, bullous pemphigoid, Darier's disease, Grover's disease, Hailey-Hailey disease, impetigo, bullous impetigo, folliculitis, MRSA (Methicillin-resistant Staphylococcus aureus), various forms of fungal skin and nail infections, Sezary syndrome, erythroderma, and for adjunct treatment of scabies, insect bites, ichthyosis vulgaris, acquired ichthyosis, shingles (herpes zoster virus), and herpes simplex virus (oral/genital herpes). The moisturizers are also used in wound healing such as acute and/or chronic ulcer, wound from burn (first degree, second degree and third degree skin burn, pressure ulcer, chronic non-healing ulcers including but not limited to pyogenic granuloma, stasis ulcer, diabetic foot ulcer, Behcet's disease, discoid lupus, lupus ulcer, various granulomatous skin/mucosal diseases such as aphthous stomatitis, necrobiosis lipoidica diabeticorum. The moisturizers are also used for post-laser procedures, post-chemical peel procedures, wound care/dressing after skin biopsy, surgical operation, lip balm, lipsticks for dry, peeling lips, lip dermatitis, dry and sensitive skin of inner groin, skin of genitalia, armpits, and on skin all over body, pediatric skin, baby moisturizer, bed sore, pressure sore, pressure-induced skin ulcer, and other skin conditions that result in dry, scaly, or itchy skin.

The moisturizer compositions above may be used alone, or in combination with one or more additional primary or supplemental agents described herein. The additional agents may be part of the same formulation or may be administered separately in different formulations. Specific, non-limiting examples are discussed further in the working examples below. The formulations may also include one or more vehicle ingredients to provide a suitable vehicle for topical administration including, but not limited to, those described above. The compositions may be processed and manufactured according to conventional methods known in the art.

The moisturizer compositions above may include moisturizing agents as discussed above in detail, and may be used as a lotion, gel, hydrogel, solution, foam, cream, ointment, shampoo, or other suitable vehicle alone or with a dressing such as xeroform, gauze, film, wipe, bandage, band-aids, hydrocolloids, alginates, and the like.

### Example 2: Retinoid-Based Topical Compositions

Tables 2-4 below include exemplary formulations for topical compositions that include at least one retinoid as a primary agent. The topical compositions may be applied to a user's body for any indication that may benefit from treatment with a retinoid, including those indications described above. Specific non-limiting examples of indications that may be treated using a retinoid-based topical composition are discussed in additional examples below. Additional active and/or non-active supplemental agents may also be included in or used in conjunction with the formulations below in accordance with the embodiments described herein. For example, the formulations in Tables 2-4 may also include or be administered in conjunction with, Pterostilbene or its derivatives (trans-3,5-dimethoxy-4-hydroxystilbene) (01%-100%, 1 mg-1000 mg, 1.0 μM-500 μM, 1.0 μg/mg-1000 μg/mL), Equol (4′,7-isoflavandiol) (R-equol, racemic equol or S-equol (l %-25%, 1 nM-25 nM, 1 mg, 2.5 mg, 5 mg, 7.5 mg, 10 mg, 15 mg, 20 mg, 25 mg, 30 mg, 40 mg, 50 mg-100 mg. The formulations may also include one or more vehicle ingredients to provide a suitable vehicle for topical administration including, but not limited to, those described above. The compositions may be processed and manufactured according to conventional methods known in the art.

TABLE 2
Active ingredient formulations for topical retinol-based topical compositions.
Each formulation below includes retinol as a primary agent. The formulations
below may be used in conjunction with or further include one or more of
the additional supplemental agents disclosed above. Additionally, retinol
(at concentrations specified herein) may be used alone or in combination
with one or more of the supplemental agents disclosed above.

| Formulation No. | Ingredients (% w/w or w/v, or as indicated) |
|---|---|
| 91 | Suberin (3%) |
| | Retinol (0.5%-3%) |
| 92 | Suberin (3%) |
| | Retinol (0.5%) |
| 93 | Suberin (3%) |
| | Retinol (1%) |
| 94 | Suberin (3%) |
| | Retinol (1.5%) |
| 95 | Suberin (3%) |
| | Retinol (2%) |
| 96 | Suberin (3%) |
| | Retinol (2.5%) |
| 97 | Suberin (3%) |
| | Retinol (3%) |
| 98 | Cutin (1%-5%) |
| | Retinol (0.05%-3%) |
| 99 | Cutin (1%, 2%, 3%, 4%, or 5%) |
| | Retinol (0.05%) |
| 100 | Cutin (1%, 2%, 3%, 4%, or 5%) |
| | Retinol (1%) |
| 101 | Cutin (1%, 2%, 3%, 4%, or 5%) |
| | Retinol (1.5%) |
| 102 | Cutin (1%, 2%, 3%, 4%, or 5%) |
| | Retinol (2%) |
| 103 | Cutin (1%, 2%, 3%, 4%, or 5%) |
| | Retinol (2.5%) |
| 104 | Cutin (1%, 2%, 3%, 4%, or 5%) |
| | Retinol (3%) |
| 105 | Astaxanthin (1%-10%) |
| | Retinol (0.5%-3%) |
| 106 | Astaxanthin (1%, 2%, 3%, 4%, 5%, 7.5%, or 10%) |
| | Retinol (0.5%) |
| 107 | Astaxanthin (1%, 2%, 3%, 4%, 5%, 7.5%, or 10%) |
| | Retinol (1%) |
| 108 | Astaxanthin (1%, 2%, 3%, 4%, 5%, 7.5%, or 10%) |
| | Retinol (1.5%) |
| 109 | Astaxanthin (1%, 2%, 3%, 4%, 5%, 7.5%, or 10%) |
| | Retinol (2%) |
| 110 | Astaxanthin (1%, 2%, 3%, 4%, 5%, 7.5%, or 10%) |
| | Retinol (2.5%) |
| 111 | Astaxanthin (1%, 2%, 3%, 4%, 5%, 7.5%, or 10%) |
| | Retinol (3%) |
| 112 | Arvelexin (10 μM-200 μM) |
| | Retinol (0.05%-3%) |
| 113 | Arvelexin (10 μM, 25 μM, 50 μM, 75 μM, 100 μM, 150 μM, or 200 μM) |
| | Retinol (0.05%) |
| 114 | Arvelexin (10 μM, 25 μM, 50 μM, 75 μM, 100 μM, 150 μM, or 200 μM) |
| | Retinol (0.1%) |
| 115 | Arvelexin (10 μM, 25 μM, 50 μM, 75 μM, 100 μM, 150 μM, or 200 μM) |
| | Retinol (0.5%) |
| 116 | Arvelexin (10 μM, 25 μM, 50 μM, 75 μM, 100 μM, 150 μM, or 200 μM) |
| | Retinol (1%) |
| 117 | Arvelexin (10 μM, 25 μM, 50 μM, 75 μM, 100 μM, 150 μM, or 200 μM) |
| | Retinol (1.5%) |
| 118 | Arvelexin (10 μM, 25 μM, 50 μM, 75 μM, 100 μM, 150 μM, or 200 μM) |
| | Retinol (2%) |
| 119 | Arvelexin (10 μM, 25 μM, 50 μM, 75 μM, 100 μM, 150 μM, or 200 μM) |
| | Retinol (2.5%) |
| 120 | Arvelexin (10 μM, 25 μM, 50 μM, 75 μM, 100 μM, 150 μM, or 200 μM) |
| | Retinol (3%) |
| 121 | Suberin (3%) |
| | Astaxanthin (1%-10%) |
| | Retinol (0.05%-3%) |
| 122 | Suberin (3%) |
| | Astaxanthin (1%, 2%, 3%, 4%, 5%, 7.5%, or 10%) |
| | Retinol (0.05%) |
| 123 | Suberin (3%) |
| | Astaxanthin (1%, 2%, 3%, 4%, 5%, 7.5%, or 10%) |
| | Retinol (0.1%) |
| 124 | Suberin (3%) |
| | Astaxanthin (1%, 2%, 3%, 4%, 5%, 7.5%, or 10%) |
| | Retinol (0.5%) |
| 125 | Suberin (3%) |
| | Astaxanthin (1%, 2%, 3%, 4%, 5%, 7.5%, or 10%) |
| | Retinol (1%) |
| 126 | Suberin (3%) |
| | Astaxanthin (1%, 2%, 3%, 4%, 5%, 7.5%, or 10%) |
| | Retinol (1.5%) |
| 127 | Suberin (3%) |
| | Astaxanthin (1%, 2%, 3%, 4%, 5%, 7.5%, or 10%) |
| | Retinol (2%) |
| 128 | Suberin (3%) |
| | Astaxanthin (1%, 2%, 3%, 4%, 5%, 7.5%, or 10%) |
| | Retinol (2.5%) |
| 129 | Suberin (3%) |
| | Astaxanthin (1%, 2%, 3%, 4%, 5%, 7.5%, or 10%) |
| | Retinol (3%) |
| 130 | Suberin (3%) |
| | Arvelexin (10 μM-200 μM) |
| | Retinol (0.05%-3%) |
| 131 | Suberin (3%) |
| | Arvelexin (10 μM, 25 μM, 50 μM, 75 μM, 100 μM, 150 μM, or 200 μM) |
| | Retinol (0.05%) |
| 132 | Suberin (3%) |
| | Arvelexin (10 μM, 25 μM, 50 μM, 75 μM, 100 μM, 150 μM, or 200 μM) |
| | Retinol (0.1%) |
| 133 | Suberin (3%) |
| | Arvelexin (10 μM, 25 μM, 50 μM, 75 μM, 100 μM, 150 μM, or 200 μM) |
| | Retinol (0.5%) |
| 134 | Suberin (3%) |
| | Arvelexin (10 μM, 25 μM, 50 μM, 75 μM, 100 μM, 150 μM, or 200 μM) |
| | Retinol (1%) |
| 135 | Suberin (3%) |
| | Arvelexin (10 μM, 25 μM, 50 μM, 75 μM, 100 μM, 150 μM, or 200 μM) |
| | Retinol (1.5%) |
| 136 | Suberin (3%) |
| | Arvelexin (10 μM, 25 μM, 50 μM, 75 μM, 100 μM, 150 μM, or 200 μM) |
| | Retinol (2%) |
| 137 | Suberin (3%) |
| | Arvelexin (10 μM, 25 μM, 50 μM, 75 μM, 100 μM, 150 μM, or 200 μM) |
| | Retinol (2.5%) |
| 138 | Suberin (3%) |
| | Arvelexin (10 μM, 25 μM, 50 μM, 75 μM, 100 μM, 150 μM, or 200 μM) |
| | Retinol (3%) |
| 139 | Suberin (3%) |
| | Retinol (5.5%) (peel) |
| 140 | Cutin (1%-5%) |
| | Retinol (5.5%) (peel) |
| 141 | Cutin (1%) |
| | Retinol 5.5% (peel) |
| 142 | Cutin (2%) |
| | Retinol 5.5% (peel) |
| 143 | Cutin (3%) |
| | Retinol 5.5% (peel) |
| 144 | Cutin (4%) |
| | Retinol 5.5% (peel) |
| 145 | Cutin (5%) |
| | Retinol 5.5% (peel) |
| 146 | Cutin (1%-5%) |
| | Astaxanthin (1%-10%) |
| | Retinol (0.05%-3%) |
| 147 | Cutin (1%, 2%, 3%, 4%, or 5%) |
| | Astaxanthin (1%, 2%, 3%, 4%, 5%, 7.5%, or 10%) |
| | Retinol (0.05%) |
| 148 | Cutin (1%, 2%, 3%, 4%, or 5%) |
| | Astaxanthin (1%, 2%, 3%, 4%, 5%, 7.5%, or 10%) |
| | Retinol (0.1%) |
| 149 | Cutin (1%, 2%, 3%, 4%, or 5%) |
| | Astaxanthin (1%, 2%, 3%, 4%, 5%, 7.5%, or 10%) |
| | Retinol (0.5%) |
| 150 | Cutin (1%, 2%, 3%, 4%, or 5%) |
| | Astaxanthin (1%, 2%, 3%, 4%, 5%, 7.5%, or 10%) |
| | Retinol (1%) |
| 151 | Cutin (1%, 2%, 3%, 4%, or 5%) |
| | Astaxanthin (1%, 2%, 3%, 4%, 5%, 7.5%, or 10%) |
| | Retinol (1.5%) |
| 152 | Cutin (1%, 2%, 3%, 4%, or 5%) |
| | Astaxanthin (1%, 2%, 3%, 4%, 5%, 7.5%, or 10%) |
| | Retinol (2%) |
| 153 | Cutin (1%, 2%, 3%, 4%, or 5%) |
| | Astaxanthin (1%, 2%, 3%, 4%, 5%, 7.5%, or 10%) |
| | Retinol (2.5%) |
| 154 | Cutin (1%, 2%, 3%, 4%, or 5%) |
| | Astaxanthin (1%, 2%, 3%, 4%, 5%, 7.5%, or 10%) |
| | Retinol (3%) |
| 155 | Cutin (1%-5.5%) |
| | Arvelexin (10 μM-200 μM) |
| | Retinol (0.05%-3%) |
| 156 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (0.05%) |
| 157 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (0.1%) |
| 158 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (0.5%) |
| 159 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (1%) |
| 160 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (1.5%) |
| 161 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (2%) |
| 162 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (2.5%) |
| 163 | Cutin (1%, 2%, 3%, 4%, 5%, or 5.5%) |
| | Arvelexin (10 μM, 25 μM, 50 μM, 75 μM, 100 μM, 150 μM, or 200 μM) |
| | Retinol (3%) |
| 164 | Astaxanthin (1%-10%) |
| | Arvelexin (10 μM-200 μM) |
| | Retinol (0.05%-3%) |
| 165 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (0.05%) |
| 166 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (0.1%) |
| 167 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (0.5%) |
| 168 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (1%) |
| 169 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (1.5%) |
| 170 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (2%) |
| 171 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (2.5%) |
| 172 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Retinol (3%) |
| 173 | Lignin (1%-30%) |
| | Retinol (0.05%-3%) |
| 174 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (0.05%) |
| 175 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (0.1%) |
| 176 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (0.5%) |
| 177 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (1%) |
| 178 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (1.5%) |
| 179 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (2%) |
| 180 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (2.5%) |
| 181 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Retinol (3%) |
| 182 | Lignin (1%-30%) |
| | Astaxanthin (1%-10%) |
| | Retinol (0.05%-3%) |
| 183 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (0.05%) |
| 184 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (0.1%) |
| 185 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (0.5%) |
| 186 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (1%) |
| 187 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (1.5%) |
| 188 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (2%) |
| 189 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (2.5%) |
| 190 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Retinol (3%) |
| 191 | Lignin (1%-30%) |
| | Arvelexin (10 μM-200 μM) |
| | Retinol (0.05%-3%) |
| 192 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (0.05%) |
| 193 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (0.1%) |
| 194 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (0.5%) |
| 195 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (1%) |
| 196 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (1.5%) |
| 197 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (2%) |
| 198 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (2.5%) |
| 199 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Retinol (3%) |

TABLE 3
Active ingredient formulations for topical retinoid-based topical compositions.
The formulations below may be used in conjunction with or further include
one or more of the additional supplemental agents disclosed above. Each
formulation below includes tretinoin as a primary agent. Additionally, Tretinoin
(at concentrations specified herein) may be used alone or in combination
with one or more of the supplemental agents disclosed above.

| Formulation No. | Ingredients (% w/w or w/v, or as indicated) |
|---|---|
| 200 | Suberin (3%) |
| | Tretinoin (0.025%-3%) |
| 201 | Suberin (3%) |
| | Tretinoin (0.025%) |
| 202 | Suberin (3%) |
| | Tretinoin (0.05%, 0.075%, 0.08%, 0.085%, 0.09%) |
| 203 | Suberin (3%) |
| | Tretinoin (0.1%, 0.15%, 0.2%, 0.5%, 0.75%, 0.80-0.9%, 1.0%) |
| 204 | Suberin (3%) |
| | Tretinoin (1%) |
| 205 | Suberin (3%) |
| | Tretinoin (1.5%) |
| 206 | Suberin (3%) |
| | Tretinoin (2%) |
| 207 | Suberin (3%) |
| | Tretinoin (2.5%) |
| 208 | Suberin (3%) |
| | Tretinoin (3%) |
| 209 | Cutin (1%-5.0) |
| | Tretinoin (0.025%-3%) |
| 210 | Cutin (1%, 2%, 3%, 4%, or 5%) |
| | Tretinoin (0.025%) |
| 211 | Cutin (1%, 2%, 3%, 4%, or 5%) |
| | Tretinoin (0.05%, 0.075%, 0.08%, 0.085%, 0.09%) |
| 212 | Cutin (1%, 2%, 3%, 4%, or 5%) |
| | Tretinoin (0.1%, 0.15%, 0.2%, 0.5%, 0.75%, 0.80-0.9%, 1.0%) |
| 213 | Cutin (1%, 2%, 3%, 4%, or 5%) |
| | Tretinoin (1%) |
| 214 | Cutin (1%, 2%, 3%, 4%, or 5%) |
| | Tretinoin (1.5%) |
| 215 | Cutin (1%, 2%, 3%, 4%, or 5%) |
| | Tretinoin (2%) |
| 216 | Cutin (1%, 2%, 3%, 4%, or 5%) |
| | Tretinoin (2.5%) |
| 217 | Cutin (1%, 2%, 3%, 4%, or 5%) |
| | Tretinoin (3%) |
| 218 | Astaxanthin (1%-10%) |
| | Tretinoin (0.025%-3%) |
| 219 | Astaxanthin (1%, 2%, 3%, 4%, 5%, 7.5%, or 10%) |
| | Tretinoin (0.025%) |
| 220 | Astaxanthin (1%, 2%, 3%, 4%, 5%, 7.5%, or 10%) |
| | Tretinoin (0.05%, 0.075%, 0.08%, 0.085%, 0.09%) |
| 221 | Astaxanthin (1%, 2%, 3%, 4%, 5%, 7.5%, or 10%) |
| | Tretinoin (0.1%, 0.15%, 0.2%, 0.5%, 0.75%, 0.80-0.9%, 1.0%) |
| 222 | Astaxanthin (1%, 2%, 3%, 4%, 5%, 7.5%, or 10%) |
| | Tretinoin (1%) |
| 223 | Astaxanthin (1%, 2%, 3%, 4%, 5%, 7.5%, or 10%) |
| | Tretinoin (1.5%) |
| 224 | Astaxanthin (1%, 2%, 3%, 4%, 5%, 7.5%, or 10%) |
| | Tretinoin (2%) |
| 225 | Astaxanthin (1%, 2%, 3%, 4%, 5%, 7.5%, or 10%) |
| | Tretinoin (2.5%) |
| 226 | Astaxanthin (1%, 2%, 3%, 4%, 5%, 7.5%, or 10%) |
| | Tretinoin (3%) |
| 227 | Suberin (3%) |
| | Astaxanthin (1%-10%) |
| | Tretinoin (0.025%-3%) |
| 228 | Suberin (3%) |
| | Astaxanthin (1%, 2%, 3%, 4%, 5%, 7.5%, or 10%) |
| | Tretinoin (0.025%) |
| 229 | Suberin (3%) |
| | Astaxanthin (1%, 2%, 3%, 4%, 5%, 7.5%, or 10%) |
| | Tretinoin (0.05%, 0.075%, 0.08%, 0.085%, 0.09%) |
| 230 | Suberin (3%) |
| | Astaxanthin (1%, 2%, 3%, 4%, 5%, 7.5%, or 10%) |
| | Tretinoin (0.1%, 0.15%, 0.2%, 0.5%, 0.75%, 0.80-0.9%, 1.0%) |
| 231 | Suberin (3%) |
| | Astaxanthin (1%, 2%, 3%, 4%, 5%, 7.5%, or 10%) |
| | Tretinoin (1%) |
| 232 | Suberin (3%) |
| | Astaxanthin (1%, 2%, 3%, 4%, 5%, 7.5%, or 10%) |
| | Tretinoin (1.5%) |
| 233 | Suberin (3%) |
| | Astaxanthin (1%, 2%, 3%, 4%, 5%, 7.5%, or 10%) |
| | Tretinoin (2%) |
| 234 | Suberin (3%) |
| | Astaxanthin (1%, 2%, 3%, 4%, 5%, 7.5%, or 10%) |
| | Tretinoin (2.5%) |
| 235 | Suberin (3%) |
| | Astaxanthin (1%, 2%, 3%, 4%, 5%, 7.5%, or 10%) |
| | Tretinoin (3%) |
| 236 | Cutin (1%-5%) |
| | Astaxanthin (1%-10%) |
| | Tretinoin (0.025%-3%) |
| 237 | Cutin (1%, 2%, 3%, 4%, or 5%) |
| | Astaxanthin (1%, 2%, 3%, 4%, 5%, 7.5%, or 10%) |
| | Tretinoin (0.025%) |
| 238 | Cutin (1%, 2%, 3%, 4%, or 5%) |
| | Astaxanthin (1%, 2%, 3%, 4%, 5%, 7.5%, or 10%) |
| | Tretinoin (0.05%, 0.075%, 0.08%, 0.085%, 0.09%) |
| 239 | Cutin (1%, 2%, 3%, 4%, or 5%) |
| | Astaxanthin (1%, 2%, 3%, 4%, 5%, 7.5%, or 10%) |
| | Tretinoin (0.1%, 0.15%, 0.2%, 0.5%, 0.75%, 0.80-0.9%, 1.0%) |
| 240 | Cutin (1%, 2%, 3%, 4%, or 5%) |
| | Astaxanthin (1%, 2%, 3%, 4%, 5%, 7.5%, or 10%) |
| | Tretinoin (1%) |
| 241 | Cutin (1%, 2%, 3%, 4%, or 5%) |
| | Astaxanthin (1%, 2%, 3%, 4%, 5%, 7.5%, or 10%) |
| | Tretinoin (1.5%) |
| 242 | Cutin (1%, 2%, 3%, 4%, or 5%) |
| | Astaxanthin (1%, 2%, 3%, 4%, 5%, 7.5%, or 10%) |
| | Tretinoin (2%) |
| 243 | Cutin (1%, 2%, 3%, 4%, or 5%) |
| | Astaxanthin (1%, 2%, 3%, 4%, 5%, 7.5%, or 10%) |
| | Tretinoin (2.5%) |
| 244 | Cutin (1%, 2%, 3%, 4%, or 5%) |
| | Astaxanthin (1%, 2%, 3%, 4%, 5%, 7.5%, or 10%) |
| | Tretinoin (3%) |
| 245 | Arvelexin (10 μM-200 μM) |
| | Tretinoin (0.025%-3%) |
| 246 | Arvelexin (10 μM, 25 μM, 50 μM, 75 μM, 100 μM, 150 μM, or 200 μM) |
| | Tretinoin (0.025%) |
| 247 | Arvelexin (10 μM, 25 μM, 50 μM, 75 μM, 100 μM, 150 μM, or 200 μM) |
| | Tretinoin (0.05%, 0.075%, 0.08%, 0.085%, 0.09%) |
| 248 | Arvelexin (10 μM, 25 μM, 50 μM, 75 μM, 100 μM, 150 μM, or 200 μM) |
| | Tretinoin (0.1%, 0.15%, 0.2%, 0.5%, 0.75%, 0.80-0.9%, 1.0%) |
| 249 | Arvelexin (10 μM, 25 μM, 50 μM, 75 μM, 100 μM, 150 μM, or 200 μM) |
| | Tretinoin (1%) |
| 250 | Arvelexin (10 μM, 25 μM, 50 μM, 75 μM, 100 μM, 150 μM, or 200 μM) |
| | Tretinoin (1.5%) |
| 251 | Arvelexin (10 μM, 25 μM, 50 μM, 75 μM, 100 μM, 150 μM, or 200 μM) |
| | Tretinoin (2%) |
| 252 | Arvelexin (10 μM, 25 μM, 50 μM, 75 μM, 100 μM, 150 μM, or 200 μM) |
| | Tretinoin (2.5%) |
| 253 | Arvelexin (10 μM, 25 μM, 50 μM, 75 μM, 100 μM, 150 μM, or 200 μM) |
| | Tretinoin (3%) |
| 254 | Suberin (3%) |
| | Arvelexin (10 μM-200 μM) |
| | Tretinoin (0.025%-3%) |
| 255 | Suberin (3%) |
| | Arvelexin (10 μM, 25 μM, 50 μM, 75 μM, 100 μM, 150 μM, or 200 μM) |
| | Tretinoin (0.025%) |
| 256 | Suberin (3%) |
| | Arvelexin (10 μM, 25 μM, 50 μM, 75 μM, 100 μM, 150 μM, or 200 μM) |
| | Tretinoin (0.05%, 0.075%, 0.08%, 0.085%, 0.09%) |
| 257 | Suberin (3%) |
| | Arvelexin (10 μM, 25 μM, 50 μM, 75 μM, 100 μM, 150 μM, or 200 μM) |
| | Tretinoin (0.1%, 0.15%, 0.2%, 0.5%, 0.75%, 0.80-0.9%, 1.0%) |
| 258 | Suberin (3%) |
| | Arvelexin (10 μM, 25 μM, 50 μM, 75 μM, 100 μM, 150 μM, or 200 μM) |
| | Tretinoin (1%) |
| 259 | Suberin (3%) |
| | Arvelexin (10 μM, 25 μM, 50 μM, 75 μM, 100 μM, 150 μM, or 200 μM) |
| | Tretinoin (1.5%) |
| 260 | Suberin (3%) |
| | Arvelexin (10 μM, 25 μM, 50 μM, 75 μM, 100 μM, 150 μM, or 200 μM) |
| | Tretinoin (2%) |
| 261 | Suberin (3%) |
| | Arvelexin (10 μM, 25 μM, 50 μM, 75 μM, 100 μM, 150 μM, or 200 μM) |
| | Tretinoin (2.5%) |
| 262 | Suberin (3%) |
| | Arvelexin (10 μM, 25 μM, 50 μM, 75 μM, 100 μM, 150 μM, or 200 μM) |
| | Tretinoin (3%) |
| 263 | Cutin (1%-5%) |
| | Arvelexin (10 μM-200 μM) |
| | Tretinoin (0.025%-3%) |
| 264 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (0.025%) |
| 265 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (0.05%,0.075%, 0.08%, 0.085%, 0.09%) |
| 266 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (0.1%, 0.15%, 0.2%, 0.5%, 0.75%, 0.80-0.9%, 1.0%) |
| 267 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (1%) |
| 268 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (1.5%) |
| 269 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (2%) |
| 270 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (2.5%) |
| 271 | Cutin (1%, 2%, 3%, 4%, or 5%) |
| | Arvelexin (10 μM, 25 μM, 50 μM, 75 μM, 100 μM, 150 μM, or 200 μM) |
| | Tretinoin (3%) |
| 272 | Astaxanthin (1%-10%) |
| | Arvelexin (10 μM-200 μM) |
| | Tretinoin (0.025%-3%) |
| 273 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (0.025%) |
| 274 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (0.05%, 0.075%, 0.08%, 0.085%, 0.09%) |
| 275 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (0.1%, 0.15%, 0.2%, 0.5%, 0.75%, 0.80-0.9%, 1.0%) |
| 276 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (1%) |
| 277 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (1.5%) |
| 278 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (2%) |
| 279 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (2.5%) |
| 280 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (3%) |
| 281 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (0.025%-3%) |
| 282 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (0.025%) |
| 283 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (0.05%, 0.075%, 0.08%, 0.085%, 0.09%) |
| 284 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (0.1%, 0.15%, 0.2%, 0.5%, 0.75%, 0.80-0.9%, 1.0%) |
| 285 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (1%) |
| 286 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (1.5%) |
| 287 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (2%) |
| 288 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (2.5%) |
| 289 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Tretinoin (3%) |
| 290 | Lignin (1%-30%) |
| | Astaxanthin (1%-10%) |
| | Tretinoin (0.025%-3%) |
| 291 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Tretinoin (0.025%) |
| 292 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Tretinoin (0.05%, 0.075%, 0.08%, 0.085%, 0.09%) |
| 293 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Tretinoin (0.1%, 0.15%, 0.2%, 0.5%, 0.75%, 0.80-0.9%, 1.0%) |
| 294 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Tretinoin (1%) |
| 295 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Tretinoin (1.5%) |
| 296 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 03 or 3%, 4%, 5%, 7.5%,10%) |
| | Tretinoin (2%) |
| 297 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Tretinoin (2.5%) |
| 298 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Tretinoin (3%) |
| 299 | Lignin (1%-30%) |
| | Arvelexin (10 μM-200 μM) |
| | Tretinoin (0.025%-3%) |
| 300 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (0.025%) |
| 301 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (0.05%, 0.075%, 0.08%, 0.085%, 0.09%) |
| 302 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (0.1%, 0.15%, 0.2%, 0.5%, 0.75%, 0.80-0.9%, 1.0%) |
| 303 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (1%) |
| 304 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (1.5%) |
| 305 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (2%) |
| 306 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (2.5%) |
| 307 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tretinoin (3%) |

TABLE 4
Active ingredient formulations for topical retinoid-based topical compositions.
Each formulation below includes tazarotene as a primary agent. The formulations
below may be used in conjunction with or further include one or more of the
additional supplemental agents disclosed above. Additionally, Tazarotene
(at concentrations specified herein) may be used alone or in combination
with one or more of the supplemental agents disclosed above.

| Formulation No. | Ingredients (% w/w or w/v, or as indicated) |
|---|---|
| 308 | Suberin (3%) |
| | Tazarotene (0.001%-1%) |
| 309 | Suberin (3%) |
| | Tazarotene (0.001%) |
| 310 | Suberin (3%) |
| | Tazarotene (0.01%) |
| 311 | Suberin (3%) |
| | Tazarotene (0.1%) |
| 312 | Suberin (3%) |
| | Tazarotene (0.25%) |
| 313 | Suberin (3%) |
| | Tazarotene (0.5%) |
| 314 | Suberin (3%) |
| | Tazarotene (0.75%) |
| 315 | Suberin (3%) |
| | Tazarotene (1%) |
| 316 | Cutin (1%-5.0), |
| | Tazarotene (0.001%-1%) |
| 317 | Cutin (1%, 2%, 3%, 4%, or 5%) |
| | Tazarotene (0.001%) |
| 318 | Cutin (1%, 2%, 3%, 4%, or 5%) |
| | Tazarotene (0.01%) |
| 319 | Cutin (1%, 2%, 3%, 4%, or 5%) |
| | Tazarotene (0.1%) |
| 320 | Cutin (1%, 2%, 3%, 4%, or 5%) |
| | Tazarotene (0.25%) |
| 321 | Cutin (1%, 2%, 3%, 4%, or 5%) |
| | Tazarotene (0.5%) |
| 322 | Cutin (1%, 2%, 3%, 4%, or 5%) |
| | Tazarotene (0.75%) |
| 323 | Cutin (1%, 2%, 3%, 4%, or 5%) |
| | Tazarotene (1%) |
| 324 | Astaxanthin (1%-3%) |
| | Tazarotene (0.001%-1%) |
| 325 | Astaxanthin (1%, 2%, or 3%) |
| | Tazarotene (0.001%) |
| 326 | Astaxanthin (1%, 2%, or 3%) |
| | Tazarotene (0.01%) |
| 327 | Astaxanthin (1%, 2%, or 3%) |
| | Tazarotene (0.1%) |
| 328 | Astaxanthin (1%, 2%, or 3%) |
| | Tazarotene (0.25%) |
| 329 | Astaxanthin (1%, 2%, or 3%) |
| | Tazarotene (0.5%) |
| 330 | Astaxanthin (1%, 2%, or 3%) |
| | Tazarotene (0.75%) |
| 331 | Astaxanthin (1%, 2%, or 3%) |
| | Tazarotene (1%) |
| 332 | Suberin (3%) |
| | Astaxanthin (1%-10%) |
| | Tazarotene (0.001%-1%) |
| 333 | Suberin (3%) |
| | Astaxanthin (1%, 2%, 3%, 4%, 5%, 7.5%, or 10%) |
| | Tazarotene (0.001%) |
| 334 | Suberin (3%) |
| | Astaxanthin (1%, 2%, 3%, 4%, 5%, 7.5%, or 10%) |
| | Tazarotene (0.01%) |
| 335 | Suberin (3%) |
| | Astaxanthin (1%, 2%, 3%, 4%, 5%, 7.5%, or 10%) |
| | Tazarotene (0.1%) |
| 336 | Suberin (3%) |
| | Astaxanthin (1%, 2%, 3%, 4%, 5%, 7.5%, or 10%) |
| | Tazarotene (0.25%) |
| 337 | Suberin (3%) |
| | Astaxanthin (1%, 2%, 3%, 4%, 5%, 7.5%, or 10%) |
| | Tazarotene (0.5%) |
| 338 | Suberin (3%) |
| | Astaxanthin (1%, 2%, 3%, 4%, 5%, 7.5%, or 10%) |
| | Tazarotene (0.75%) |
| 339 | Suberin (3%) |
| | Astaxanthin (1%, 2%, 3%, 4%, 5%, 7.5%, or 10%) |
| | Tazarotene (1%) |
| 340 | Cutin (1%-5%) |
| | Astaxanthin (1%-10%) |
| | Tazarotene (0.001%-1%) |
| 341 | Cutin (1%, 2%, 3%, 4%, or 5%) |
| | Astaxanthin (1%, 2%, 3%, 4%, 5%, 7.5%, or 10%) |
| | Tazarotene (0.001%) |
| 342 | Cutin (1%, 2%, 3%, 4%, or 5%) |
| | Astaxanthin (1%, 2%, 3%, 4%, 5%, 7.5%, or 10%) |
| | Tazarotene (0.01%) |
| 343 | Cutin (1%, 2%, 3%, 4%, or 5%) |
| | Astaxanthin (1%, 2%, 3%, 4%, 5%, 7.5%, or 10%) |
| | Tazarotene (0.1%) |
| 344 | Cutin (1%, 2%, 3%, 4%, or 5%) |
| | Astaxanthin (1%, 2%, 3%, 4%, 5%, 7.5%, or 10%) |
| | Tazarotene (0.25%) |
| 345 | Cutin (1%, 2%, 3%, 4%, or 5%) |
| | Astaxanthin (1%, 2%, 3%, 4%, 5%, 7.5%, or 10%) |
| | Tazarotene (0.5%) |
| 346 | Cutin (1%, 2%, 3%, 4%, or 5%) |
| | Astaxanthin (1%, 2%, 3%, 4%, 5%, 7.5%, or 10%) |
| | Tazarotene (0.75%) |
| 347 | Cutin (1%, 2%, 3%, 4%, or 5%) |
| | Astaxanthin (1%, 2%, 3%, 4%, 5%, 7.5%, or 10%) |
| | Tazarotene (1%) |
| 348 | Arvelexin (10 μM-200 μM) |
| | Tazarotene (0.001%-1%) |
| 349 | Arvelexin (10 μM, 25 μM, 50 μM, 75 μM, 100 μM, 150 μM, or 200 μM) |
| | Tazarotene (0.001%) |
| 350 | Arvelexin (10 μM, 25 μM, 50 μM, 75 μM, 100 μM, 150 μM, or 200 μM) |
| | Tazarotene (0.01%) |
| 351 | Arvelexin (10 μM, 25 μM, 50 μM, 75 μM, 100 μM, 150 μM, or 200 μM) |
| | Tazarotene (0.1%) |
| 352 | Arvelexin (10 μM, 25 μM, 50 μM, 75 μM, 100 μM, 150 μM, or 200 μM) |
| | Tazarotene (0.25%) |
| 353 | Arvelexin (10 μM, 25 μM, 50 μM, 75 μM, 100 μM, 150 μM, or 200 μM) |
| | Tazarotene (0.5%) |
| 354 | Arvelexin (10 μM, 25 μM, 50 μM, 75 μM, 100 μM, 150 μM, or 200 μM) |
| | Tazarotene (0.75%) |
| 355 | Arvelexin (10 μM, 25 μM, 50 μM, 75 μM, 100 μM, 150 μM, or 200 μM) |
| | Tazarotene (1%) |
| 356 | Suberin (3%) |
| | Arvelexin (10 μM-200 μM) |
| | Tazarotene (0.001%-1%) |
| 357 | Suberin (3%) |
| | Arvelexin (10 μM, 25 μM, 50 μM, 75 μM, 100 μM, 150 μM, or 200 μM) |
| | Tazarotene (0.001%) |
| 358 | Suberin (3%) |
| | Arvelexin (10 μM, 25 μM, 50 μM, 75 μM, 100 μM, 150 μM, or 200 μM) |
| | Tazarotene (0.01%) |
| 359 | Suberin (3%) |
| | Arvelexin (10 μM, 25 μM, 50 μM, 75 μM, 100 μM, 150 μM, or 200 μM) |
| | Tazarotene (0.1%) |
| 360 | Suberin (3%) |
| | Arvelexin (10 μM, 25 μM, 50 μM, 75 μM, 100 μM, 150 μM, or 200 μM) |
| | Tazarotene (0.25%) |
| 361 | Suberin (3%) |
| | Arvelexin (10 μM, 25 μM, 50 μM, 75 μM, 100 μM, 150 μM, or 200 μM) |
| | Tazarotene (0.5%) |
| 362 | Suberin (3%) |
| | Arvelexin (10 μM, 25 μM, 50 μM, 75 μM, 100 μM, 150 μM, or 200 μM) |
| | Tazarotene (0.75%) |
| 363 | Suberin (3%) |
| | Arvelexin (10 μM, 25 μM, 50 μM, 75 μM, 100 μM, 150 μM, or 200 μM) |
| | Tazarotene (1%) |
| 364 | Cutin (1%-5%) |
| | Arvelexin (10 μM-200 μM) |
| | Tazarotene (0.001%-1%) |
| 365 | Cutin (1%, 2%, 3%, 4%, or 5%) |
| | Arvelexin (10 μM, 25 μM, 50 μM, 75 μM, 100 μM, 150 μM, or 200 μM) |
| | Tazarotene (0.001%) |
| 366 | Cutin (1%, 2%, 3%, 4%, or 5%) |
| | Arvelexin (10 μM, 25 μM, 50 μM, 75 μM, 100 μM, 150 μM, or 200 μM) |
| | Tazarotene (0.01%) |
| 367 | Cutin (1%, 2%, 3%, 4%, or 5%) |
| | Arvelexin (10 μM, 25 μM, 50 μM, 75 μM, 100 μM, 150 μM, or 200 μM) |
| | Tazarotene (0.1%) |
| 368 | Cutin (1%, 2%, 3%, 4%, or 5%) |
| | Arvelexin (10 μM, 25 μM, 50 μM, 75 μM, 100 μM, 150 μM, or 200 μM) |
| | Tazarotene (0.25%) |
| 369 | Cutin (1%, 2%, 3%, 4%, or 5%) |
| | Arvelexin (10 μM, 25 μM, 50 μM, 75 μM, 100 μM, 150 μM, or 200 μM) |
| | Tazarotene (0.5%) |
| 370 | Cutin (1%, 2%, 3%, 4%, or 5%) |
| | Arvelexin (10 μM, 25 μM, 50 μM, 75 μM, 100 μM, 150 μM, or 200 μM) |
| | Tazarotene (0.75%) |
| 371 | Cutin (1%, 2%, 3%, 4%, or 5%) |
| | Arvelexin (10 μM, 25 μM, 50 μM, 75 μM, 100 μM, 150 μM, or 200 μM) |
| | Tazarotene (1%) |
| 372 | Astaxanthin (1.0-10%) |
| | Arvelexin (10 μM-200 μM) |
| | Tazarotene (0.001%-1%) |
| 373 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tazarotene (0.001%) |
| 374 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tazarotene (0.01%) |
| 375 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tazarotene (0.1%) |
| 376 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tazarotene (0.25%) |
| 377 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tazarotene (0.5%) |
| 378 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tazarotene (0.75%) |
| 379 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tazarotene (1%) |
| 380 | Lignin (15-30%) |
| | Tazarotene (0.001%-1%) |
| 381 | Lignin (15%, 16%, 17%, 18%, 19%, 20%, 21%, 22%, 23%, 24%, 25%, |
| | 26%, 27%, 28%, 29%, or 30%) |
| | Tazarotene (0.001%) |
| 382 | Lignin (15%, 16%, 17%, 18%, 19%, 20%, 21%, 22%, 23%, 24%, 25%, |
| | 26%, 27%, 28%, 29%, or 30%) |
| | Tazarotene (0.01%) |
| 383 | Lignin (15%, 16%, 17%, 18%, 19%, 20%, 21%, 22%, 23%, 24%, 25%, |
| | 26%, 27%, 28%, 29%, or 30%) |
| | Tazarotene (0.1%) |
| 384 | Lignin (15%, 16%, 17%, 18%, 19%, 20%, 21%, 22%, 23%, 24%, 25%, |
| | 26%, 27%, 28%, 29%, or 30%) |
| | Tazarotene (0.25%) |
| 385 | Lignin (15%, 16%, 17%, 18%, 19%, 20%, 21%, 22%, 23%, 24%, 25%, |
| | 26%, 27%, 28%, 29%, or 30%) |
| | Tazarotene (0.5%) |
| 386 | Lignin (15%, 16%, 17%, 18%, 19%, 20%, 21%, 22%, 23%, 24%, 25%, |
| | 26%, 27%, 28%, 29%, or 30%) |
| | Tazarotene (0.75%) |
| 387 | Lignin (15%, 16%, 17%, 18%, 19%, 20%, 21%, 22%, 23%, 24%, 25%, |
| | 26%, 27%, 28%, 29%, or 30%) |
| | Tazarotene (1%) |
| 388 | Lignin (15-30%) |
| | Astaxanthin (1%-10%) |
| | Tazarotene (0.001%-1%) |
| 389 | Lignin (15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| | Tazarotene (0.001%) |
| 390 | Lignin (15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| | Tazarotene (0.01%) |
| 391 | Lignin (15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| | Tazarotene (0.1%) |
| 392 | Lignin (15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| | Tazarotene (0.25%) |
| 393 | Lignin (15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| | Tazarotene (0.5%) |
| 394 | Lignin (15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| | Tazarotene (0.75%) |
| 395 | Lignin (15%, 16%, 17%, 18%, 19%, 20%, 21%, 22%, 23%, 24%, 25%, |
| | 26%, 27%, 28%, 29%, or 30%) |
| | Astaxanthin (1%, 2%, 3%, 4%, 5%, 7.5%, or 10%) |
| | Tazarotene (1%) |
| 396 | Lignin (1%-30%) |
| | Arvelexin (10 μM-200 μM) |
| | Tazarotene (0.001%-1%) |
| 397 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tazarotene (0.001%) |
| 398 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tazarotene (0.01%) |
| 399 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tazarotene (0.1%) |
| 400 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tazarotene (0.25%) |
| 401 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tazarotene (0.5%) |
| 402 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tazarotene (0.75%) |
| 403 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Tazarotene (1%) |

### Example 3: Retinoid-Based Oral Compositions

The use of oral retinoids (Isotretinoin and Acitretin, etretinate, bexarotene) is used in treatment of acne vulgaris, cystic acne, acne conglobata and acne rosacea, hidradenitis suppurativa and for the treatment of keratinization disorders (e.g., ichthyosis, Pityriasis rubra pilaris, and Darier's disease, Grover's disease, plantar hyperkeratosis, pitted keratolysis, keratosis pilaris), acanthosis nigricans, carcinoma (e.g., basal cell and squamous cell carcinomas, cutaneous T-cell lymphoma(mycosis fungoides), precancerous actinic keratosis, porokeratosis, Bowenoid papulosis, keratoacanthoma. These oral retinoids have many side effects including hyperlipidemia, and effects on bones causing bone resorption.

Astaxanthin has hypolipidemic properties as well as provides protection against bone resorption and loss of bone density. Therefore, such combinations will provide a safer long-term alternative to when using each of the retinoids alone.

Table 5 below includes exemplary formulations for oral compositions that include at least one retinoid as a primary agent. The oral compositions may be administered to a user for any indication that may benefit from treatment with a retinoid, including those indications described above. Additional active and/or non-active supplemental agents may also be included in or used in conjunction with the formulations below in accordance with the embodiments described herein. The formulations may also include one or more vehicle ingredients to provide a suitable vehicle for topical administration including, but not limited to, those described above. The compositions may be processed and manufactured according to conventional methods known in the art.

TABLE 5
Active ingredient formulations for
oral retinoid-based compositions.

| Formulation No. | Ingredients (dose) |
|---|---|
| 404 | Astaxanthin (1 mg-28 mg) |
| | Acitretin (1 mg-50 mg) |
| 405 | Astaxanthin (1 mg-28 mg) |
| | Isotretinoin (1 mg-50 mg) |
| 406 | Astaxanthin (1 mg-28 mg) |
| | Etretinate (1 mg-50 mg) |
| 407 | Astaxanthin (1 mg-28 mg) |
| | Bexarotene (1 mg-50 mg) |

### Example 4: Topical Compositions—Exfoliating and Keratolytic Agents

Tables 6-9 below include exemplary formulations for topical compositions that include at least one exfoliant or keratolytic agent (e.g., alpha hydroxy acids such as glycolic acid and lactic acid (and salts thereof), beta hydroxy acids such as salicylic acid (and salts thereof), and urea) and at least one primary agent (e.g., a plant-derived biopolymer (or extract thereof), arvelexin, astaxanthin). The topical compositions may be applied to a user's body for any indication that may benefit from treatment with an exfoliant or keratolytic agent (e.g., in anti-aging formulations and for treatment of seborrheic dermatitis, psoriasis vulgaris, acne vulgaris, keratosis pilaris, hyperkeratosis conditions, acanthosis nigricans, ichthyosis, xerosis, asteatotic eczema, atopic dermatitis, asteatosis cutis, seborrheic keratosis, palmoplantar hyperkeratosis, pitted keratolysis, calluses, corns, and warts), onychomycosis, onychogryphosis, including those indications described above. Specific non-limiting examples of indications that may be treated using a retinoid-based topical composition are discussed in additional examples below. Additional active and/or non-active primary or supplemental agents may also be included in or used in conjunction with the formulations below in accordance with the disclosure above. For example, the formulations in Tables 6-9 may also include or be administered in conjunction with Pterostilbene or its derivatives (trans-3,5-dimethoxy-4-hydroxystilbene) (01%-100%, 1 mg-1000 mg, 1.0 μM-500 μM, 1.0 μg/mg-1000 μg/mL), Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), Quercetin (3,3′,4′5,7-Pentahydroxyflavone). The compositions may be processed and manufactured according to conventional methods known in the art.

The formulations may also include one or more vehicle ingredients to provide a suitable vehicle for topical administration including, but not limited to, those described above. The exfoliating or keratolytic topical compositions below may be in the form of a cleanser, toner, moisturizer, scrub, peel, mask, or various emollient, lotion, solution, cream, shampoo, gel, hydrogel, ointment, foam, wipe, or other suitable vehicle. With respect to formulations in the form of a moisturizer, moisturizers are generally used to keep skin moist (See Example 1 for moisturizer formulations), but moisturizers in combination of keratolytic agents like those in the formulations below (e.g., urea up to 60%, ammonium lactate up to 20%, alpha-hydroxy acid-such as glycolic acid up to 70%, beta hydroxy acid-such as salicylic acid up to 6%) is also used as a heavy duty moisturizer that at the same time gets rid of thick, dry flaky, hyperkeratotic layers of skin.

Alpha-Hydroxy Acids

Alpha-hydroxy acids can be used in lower concentrations around 1%-20% to higher concentrations of 20%-50% and even up to 70% by weight or volume, depending on the indication for which it is used. Alpha-hydroxy acids can be used as anti-aging exfoliant in the form of cleansers, toners, moisturizers, scrubs, peels, masks, various emollients, lotions, creams, gels, solution, hydrogel, ointment, foams, wipes, or other suitable vehicle or application type known in the field. The formulations in Table 6 below include glycolic acid, but other alpha-hydroxy acids can be used in similar formulations.

TABLE 6
Formulations for exfoliating or keratolytic topical compositions. Each formulation
below includes glycolic acid (an alpha hydroxy acid) and at least one other primary
agent. The formulations below may be used in conjunction with or further include
one or more of the additional supplemental agents disclosed above.

| Formulation No. | Ingredients (% w/w or w/v, or as indicated) |
|---|---|
| 408 | Suberin (3%) |
| | Glycolic Acid (1%-70%) |
| 409 | Suberin (3%) |
| | Glycolic Acid (1%-20%) |
| 410 | Suberin (3%) |
| | Glycolic Acid (20%-50%) |
| 411 | Suberin (3%) |
| | Glycolic Acid (50%-70%) |
| 412 | Suberin (3%) |
| | Glycolic Acid (1%, 2%, 4%, 6%, 8%, 10%, 12%, 15%, 20%, 30%, |
| | 40%, 50%, 60%, or 70%) |
| 413 | Cutin (1%-5%) |
| | Glycolic Acid (1%-70%) |
| 414 | Cutin (1%, 2%, 3%, 4%, or 5%) |
| | Glycolic Acid (1%-20%) |
| 415 | Cutin (1%, 2%, 3%, 4%, or 5%) |
| | Glycolic Acid (20%-50%) |
| 416 | Cutin (1%, 2%, 3%, 4%, or 5%) |
| | Glycolic Acid (50%-70%) |
| 417 | Cutin (1%, 2%, 3%, 4%, or 5%) |
| | Glycolic Acid (1%, 2%, 4%, 6%, 8%, 10%, 12%, 15%, 20%, 30%, |
| | 40%, 50%, 60%, or 70%) |
| 418 | Suberin (3%) |
| | Astaxanthin (1%-10%) |
| | Glycolic Acid (1%-70%) |
| 419 | Suberin (3%) |
| | Astaxanthin (1%, 2%, 3%, 4%, 5%, 7.5%, or 10%) |
| | Glycolic Acid (1%-20%) |
| 420 | Suberin (3%) |
| | Astaxanthin (1%, 2%, 3%, 4%, 5%, 7.5%, or 10%) |
| | Glycolic Acid (20%-50%) |
| 421 | Suberin (3%) |
| | Astaxanthin (1%, 2%, 3%, 4%, 5%, 7.5%, or 10%) |
| | Glycolic Acid (50%-70%) |
| 422 | Suberin (3%) |
| | Astaxanthin (1%, 2%, 3%, 4%, 5%, 7.5%, or 10%) |
| | Glycolic Acid (1%, 2%, 4%, 6%, 8%, 10%, 12%, 15%, 20%, 30%, |
| | 40%, 50%, 60%, or 70%) |
| 423 | Cutin (1%-5%) |
| | Astaxanthin (1%-10%) |
| | Glycolic Acid (1%-70%) |
| 424 | Cutin (1%, 2%, 3%, 4%, or 5%) |
| | Astaxanthin (1%, 2%, 3%, 4%, 5%, 7.5%, or 10%) |
| | Glycolic Acid (1%-20%) |
| 425 | Cutin (1%, 2%, 3%, 4%, or 5%) |
| | Astaxanthin (1%, 2%, 3%, 4%, 5%, 7.5%, or 10%) |
| | Glycolic Acid (20%-50%) |
| 426 | Cutin (1%, 2%, 3%, 4%, or 5%) |
| | Astaxanthin (1%, 2%, 3%, 4%, 5%, 7.5%, or 10%) |
| | Glycolic Acid (50%-70%) |
| 427 | Cutin (1%, 2%, 3%, 4%, or 5%) |
| | Astaxanthin (1%, 2%, 3%, 4%, 5%, 7.5%, or 10%) |
| | Glycolic Acid (1%, 2%, 4%, 6%, 8%, 10%, 12%, 15%, 20%, 30%, |
| | 40%, 50%, 60%, or 70%) |
| 428 | Suberin (3%) |
| | Arvelexin (10 μM-200 μM) |
| | Glycolic Acid (1%-70%) |
| 429 | Suberin (3%) |
| | Arvelexin (10 μM, 25 μM, 50 μM, 75 μM, 100 μM, 150 μM, or 200 μM) |
| | Glycolic Acid (1%-20%) |
| 430 | Suberin (3%) |
| | Arvelexin (10 μM, 25 μM, 50 μM, 75 μM, 100 μM, 150 μM, or 200 μM) |
| | Glycolic Acid (20%-50%) |
| 431 | Suberin (3%) |
| | Arvelexin (10 μM, 25 μM, 50 μM, 75 μM, 100 μM, 150 μM, or 200 μM) |
| | Glycolic Acid (50%-70%) |
| 432 | Suberin (3%) |
| | Arvelexin (10 μM, 25 μM, 50 μM, 75 μM, 100 μM, 150 μM, or 200 μM) |
| | Glycolic Acid (1%, 2%, 4%, 6%, 8%, 10%, 12%, 15%, 20%, 30%, |
| | 40%, 50%, 60%, or 70%) |
| 433 | Cutin (1%-5%) |
| | Arvelexin (10 μM-200 μM) |
| | Glycolic Acid (1%-70%) |
| 434 | Cutin (1%, 2%, 3%, 4%, or 5%) |
| | Arvelexin (10 μM, 25 μM, 50 μM, 75 μM, 100 μM, 150 μM, or 200 μM) |
| | Glycolic Acid (1%-20%) |
| 435 | Cutin (1%, 2%, 3%, 4%, or 5%) |
| | Arvelexin (10 μM, 25 μM, 50 μM, 75 μM, 100 μM, 150 μM, or 200 μM) |
| | Glycolic Acid (20%-50%) |
| 436 | Cutin (1%, 2%, 3%, 4%, or 5%) |
| | Arvelexin (10 μM, 25 μM, 50 μM, 75 μM, 100 μM, 150 μM, or 200 μM) |
| | Glycolic Acid (50%-70%) |
| 437 | Cutin (1%, 2%, 3%, 4%, or 5%) |
| | Arvelexin (10 μM, 25 μM, 50 μM, 75 μM, 100 μM, 150 μM, or 200 μM) |
| | Glycolic Acid (1%, 2%, 4%, 6%, 8%, 10%, 12%, 15%, 20%, 30%, |
| | 40%, 50%, 60%, or 70%) |
| 438 | Lignin (1%-30%) |
| | Glycolic Acid (1%-70%) |
| 439 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Glycolic Acid (1%-20%) |
| 440 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Glycolic Acid (20%-50%) |
| 441 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Glycolic Acid (50%-70%) |
| 442 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Glycolic Acid (1%, 2%, 4%, 6%, 8%, 10%, 12%, 15%, 20%, 30%, |
| | 40%, 50%, 60%, or 70%) |
| 443 | Lignin (1%-30%) |
| | Astaxanthin (1%-10%) |
| | Glycolic Acid (1%-70%) |
| 444 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Glycolic Acid (1%-20%) |
| 445 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Glycolic Acid (20%-50%) |
| 446 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Glycolic Acid (50%-70%) |
| 447 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Glycolic Acid (1%, 2%, 4%, 6%, 8%, 10%, 12%, 15%, 20%, 30%, |
| | 40%, 50%, 60%, or 70%) |
| 448 | Lignin (1%-30%) |
| | Arvelexin (10 μM, 25 μM, 50 μM, 75 μM, 100 μM, 150 μM, or 200 μM) |
| | Glycolic Acid (1%-70%) |
| 449 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Glycolic Acid (1%-20%) |
| 450 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Glycolic Acid (20%-50%) |
| 451 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Glycolic Acid (50%-70%) |
| 452 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Glycolic Acid (1%, 2%, 4%, 6%, 8%, 10%, 12%, 15%, 20%, 30%, |
| | 40%, 50%, 60%, or 70%) |

Beta Hydroxy Acids

Beta-hydroxy acids such as salicylic acid can be used in concentrations approved for over the counter used around 2%-6% (for use as an exfoliant in anti-aging formulations and for treatment of seborrheic dermatitis, psoriasis vulgaris, acne vulgaris, keratosis pilaris, acanthosis nigricans, ichthyosis, xerosis, asteatotic eczema, xerosis, palmoplantar hyperkeratosis, seborrheic keratosis, atopic dermatitis, asteatosis cutis, pitted keratolysis, calluses, corns, and warts) to higher prescribed concentrations of up to about 50% by weight or volume (for treatment of warts, calluses, corms, plantar hyperkeratosis), depending on the indication for which it is used. For treatment of warts, calluses, corns, plantar hyperkeratosis, Beta-hydroxy acids may be used in the form of cream, ointment, hydrogel, gel, solution, spray, patch, under-occlusion, sticks, etc. from M %-50%). Beta-hydroxy acids can be used as anti-aging exfoliant in the form of cleansers, rinse, shampoo, toners, moisturizers, scrubs, peels, masks, various emollients, lotions, creams, gels, hydrogel, ointment, solution, foams, wipes, or other suitable vehicle or application type known in the field. The formulations in Table 7 below include glycolic acid, but other beta-hydroxy acids can be used in similar formulations.

TABLE 7
Formulations for exfoliating or keratolytic topical compositions. Each formulation
below includes salicylic acid and at least one other primary agent. The formulations
below may be used in conjunction with or further include one or more of the
additional supplemental agents disclosed above such as Pterostilbene or its
derivatives (trans-3,5-dimethoxy-4-hydroxystilbene) (01%-100%, 1 mg-1000 mg,
1.0 μM-500 μM, 1.0 μg/mg-1000 μg/mL), Equol (4′,7-isoflavandiol)
(R-equol, racemic equol or S-equol (1%-25%, 1 nM-25 nM, 1 mg, 2.5 mg, 5 mg,
7.5 mg, 10 mg, 15 mg, 20 mg, 25 mg, 30 mg, 40 mg, 50 mg-100 mg, or 1 nM-25
nM), Quercetin (3,3′,4′5,7-Pentahydroxyflavone).

| Formulation No. | Ingredients (% w/w or w/v, or as indicated) |
|---|---|
| 453 | Suberin (3%) |
| | Salicylic Acid (2%-6%) |
| 454 | Suberin (3%) |
| | Salicylic Acid (2%) |
| 455 | Suberin (3%) |
| | Salicylic Acid (3%) |
| 456 | Suberin (3%) |
| | Salicylic Acid (4%) |
| 457 | Suberin (3%) |
| | Salicylic Acid (5%) |
| 458 | Suberin (3%) |
| | Salicylic Acid (6%) |
| 459 | Cutin (1%-5%) |
| | Salicylic Acid (2%-6%) |
| 460 | Cutin (1%, 2%, 3%, 4%, or 5%) |
| | Salicylic Acid (2%) |
| 461 | Cutin (1%, 2%, 3%, 4%, or 5%) |
| | Salicylic Acid (3%) |
| 462 | Cutin (1%, 2%, 3%, 4%, or 5%) |
| | Salicylic Acid (4%) |
| 463 | Cutin (1%, 2%, 3%, 4%, or 5%) |
| | Salicylic Acid (5%) |
| 464 | Cutin (1%, 2%, 3%, 4%, or 5%) |
| | Salicylic Acid (6%) |
| 465 | Suberin (3%) |
| | Astaxanthin (1%, 2%, 3%, 4%, 5%, 7.5%, or 10%) |
| | Salicylic Acid (2%-6%) |
| 466 | Suberin (3%) |
| | Astaxanthin (1%, 2%, 3%, 4%, 5%, 7.5%, or 10%) |
| | Salicylic Acid (2%) |
| 467 | Suberin (3%) |
| | Astaxanthin (1%, 2%, 3%, 4%, 5%, 7.5%, or 10%) |
| | Salicylic Acid (3%) |
| 468 | Suberin (3%) |
| | Astaxanthin (1%, 2%, 3%, 4%, 5%, 7.5%, or 10%) |
| | Salicylic Acid (4%) |
| 469 | Suberin (3%) |
| | Astaxanthin (1%, 2%, 3%, 4%, 5%, 7.5%, or 10%) |
| | Salicylic Acid (5%) |
| 470 | Suberin (3%) |
| | Astaxanthin (1%, 2%, 3%, 4%, 5%, 7.5%, or 10%) |
| | Salicylic Acid (6%) |
| 471 | Cutin (1%-5%) |
| | Astaxanthin (1%-10%) |
| | Salicylic Acid (2%-6%) |
| 472 | Cutin (1%, 2%, 3%, 4%, or 5%) |
| | Astaxanthin (1%, 2%, 3%, 4%, 5%, 7.5%, or 10%) |
| | Salicylic Acid (2%) |
| 473 | Cutin (1%, 2%, 3%, 4%, or 5%) |
| | Astaxanthin (1%, 2%, 3%, 4%, 5%, 7.5%, or 10%) |
| | Salicylic Acid (3%) |
| 474 | Cutin (1%, 2%, 3%, 4%, or 5%) |
| | Astaxanthin (1%, 2%, 3%, 4%, 5%, 7.5%, or 10%) |
| | Salicylic Acid (4%) |
| 475 | Cutin (1%, 2%, 3%, 4%, or 5%) |
| | Astaxanthin (1%, 2%, 3%, 4%, 5%, 7.5%, or 10%) |
| | Salicylic Acid (5%) |
| 476 | Cutin (1%, 2%, 3%, 4%, or 5%) |
| | Astaxanthin (1%, 2%, 3%, 4%, 5%, 7.5%, or 10%) |
| | Salicylic Acid (6%) |
| 477 | Suberin (3%) |
| | Arvelexin (10 μM-200 μM) |
| | Salicylic Acid (2%-6%) |
| 478 | Suberin (3%) |
| | Arvelexin (10 μM, 25 μM, 50 μM, 75 μM, 100 μM, 150 μM, or 200 μM) |
| | Salicylic Acid (2%) |
| 479 | Suberin (3%) |
| | Arvelexin (10 μM, 25 μM, 50 μM, 75 μM, 100 μM, 150 μM, or 200 μM) |
| | Salicylic Acid (3%) |
| 480 | Suberin (3%) |
| | Arvelexin (10 μM, 25 μM, 50 μM, 75 μM, 100 μM, 150 μM, or 200 μM) |
| | Salicylic Acid (4%) |
| 481 | Suberin (3%) |
| | Arvelexin (10 μM, 25 μM, 50 μM, 75 μM, 100 μM, 150 μM, or 200 μM) |
| | Salicylic Acid (5%) |
| 482 | Suberin (3%) |
| | Arvelexin (10 μM, 25 μM, 50 μM, 75 μM, 100 μM, 150 μM, or 200 μM) |
| | Salicylic Acid (6%) |
| 483 | Cutin (1%-5%) |
| | Arvelexin (10 μM-200 μM) |
| | Salicylic Acid (2%-6%) |
| 484 | Cutin (1%, 2%, 3%, 4%, or 5%) |
| | Arvelexin (10 μM, 25 μM, 50 μM, 75 μM, 100 μM, 150 μM, or 200 μM) |
| | Salicylic Acid (2%) |
| 485 | Cutin (1%, 2%, 3%, 4%, or 5%) |
| | Arvelexin (10 μM, 25 μM, 50 μM, 75 μM, 100 μM, 150 μM, or 200 μM) |
| | Salicylic Acid (3%) |
| 486 | Cutin (1%, 2%, 3%, 4%, or 5%) |
| | Arvelexin (10 μM, 25 μM, 50 μM, 75 μM, 100 μM, 150 μM, or 200 μM) |
| | Salicylic Acid (4%) |
| 487 | Cutin (1%, 2%, 3%, 4%, or 5%) |
| | Arvelexin (10 μM, 25 μM, 50 μM, 75 μM, 100 μM, 150 μM, or 200 μM) |
| | Salicylic Acid (5%) |
| 488 | Cutin (1%, 2%, 3%, 4%, or 5%) |
| | Arvelexin (10 μM, 25 μM, 50 μM, 75 μM, 100 μM, 150 μM, or 200 μM) |
| | Salicylic Acid (6%) |
| 489 | Lignin (1%-30%) |
| | Salicylic Acid (2%-6%) |
| 490 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Salicylic Acid (2%) |
| 491 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Salicylic Acid (3%) |
| 492 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Salicylic Acid (4%) |
| 493 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Salicylic Acid (5%) |
| 494 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Salicylic Acid (6%) |
| 495 | Lignin (1%-30%) |
| | Astaxanthin (1%-10%) |
| | Salicylic Acid (2%-6%) |
| 496 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Salicylic Acid (2%) |
| 497 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Salicylic Acid (3%) |
| 498 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Salicylic Acid (4%) |
| 499 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Salicylic Acid (5%) |
| 500 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Salicylic Acid (6%) |
| 501 | Lignin (1%-30%) |
| | Arvelexin (10 μM, 25 μM, 50 μM, 75 μM, 100 μM, 150 μM, or 200 μM) |
| | Salicylic Acid (2%-6%) |
| 502 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Salicylic Acid (2%) |
| 503 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Salicylic Acid (3%) |
| 504 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Salicylic Acid (4%) |
| 505 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Salicylic Acid (5%) |
| 506 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Salicylic Acid (6%) |

Ammonium Lactate

Ammonium lactate is the ammonium salt of lactic acid (an alpha-hydroxy acid) and can be used in concentrations up to about 20% by weight or volume.

TABLE 8
Formulations for exfoliating or keratolytic topical compositions. Each formulation
below includes ammonium lactate and at least one other primary agent. The
formulations below may be used in conjunction with or further include one
or more of the additional supplemental agents disclosed above.

| Formulation No. | Ingredients (% w/w or w/v, or as indicated) |
|---|---|
| 507 | Suberin (3%) |
| | Ammonium lactate (12%-20%) |
| 508 | Suberin (3%) |
| | Ammonium lactate (12%) |
| 509 | Suberin (3%) |
| | Ammonium lactate (15%) |
| 510 | Suberin (3%) |
| | Ammonium lactate (17.5%) |
| 511 | Suberin (3%) |
| | Ammonium lactate (20%) |
| 512 | Cutin (1%-5%) |
| | Ammonium lactate (12%-20%) |
| 513 | Cutin (1%, 2%, 3%, 4%, or 5%) |
| | Ammonium lactate (12%) |
| 514 | Cutin (1%, 2%, 3%, 4%, or 5%) |
| | Ammonium lactate (15%) |
| 515 | Cutin (1%, 2%, 3%, 4%, or 5%) |
| | Ammonium lactate (17.5%) |
| 516 | Cutin (1%, 2%, 3%, 4%, or 5%) |
| | Ammonium lactate (20%) |
| 517 | Suberin (3%) |
| | Astaxanthin (1%, 2%, 3%, 4%, 5%, 7.5%, or 10%) |
| | Ammonium lactate (12%-20%) |
| 518 | Suberin (3%) |
| | Astaxanthin (1%, 2%, 3%, 4%, 5%, 7.5%, or 10%) |
| | Ammonium lactate (12%) |
| 519 | Suberin (3%) |
| | Astaxanthin (1%, 2%, 3%, 4%, 5%, 7.5%, or 10%) |
| | Ammonium lactate (15%) |
| 520 | Suberin (3%) |
| | Astaxanthin (1%, 2%, 3%, 4%, 5%, 7.5%, or 10%) |
| | Ammonium lactate (17.5%) |
| 521 | Suberin (3%) |
| | Astaxanthin (1%, 2%, 3%, 4%, 5%, 7.5%, or 10%) |
| | Ammonium lactate (20%) |
| 522 | Cutin (1%-5%) |
| | Astaxanthin (1%-10%) |
| | Ammonium lactate (12%-20%) |
| 523 | Cutin (1%, 2%, 3%, 4%, or 5%) |
| | Astaxanthin (1%, 2%, 3%, 4%, 5%, 7.5%, or 10%) |
| | Ammonium lactate (12%) |
| 524 | Cutin (1%, 2%, 3%, 4%, or 5%) |
| | Astaxanthin (1%, 2%, 3%, 4%, 5%, 7.5%, or 10%) |
| | Ammonium lactate (15%) |
| 525 | Cutin (1%, 2%, 3%, 4%, or 5%) |
| | Astaxanthin (1%, 2%, 3%, 4%, 5%, 7.5%, or 10%) |
| | Ammonium lactate (17.5%) |
| 526 | Cutin (1%, 2%, 3%, 4%, or 5%) |
| | Astaxanthin (1%, 2%, 3%, 4%, 5%, 7.5%, or 10%) |
| | Ammonium lactate (20%) |
| 527 | Suberin (3%) |
| | Arvelexin (10 μM-200 μM) |
| | Ammonium lactate (12%-20%) |
| 528 | Suberin (3%) |
| | Arvelexin (10 μM, 25 μM, 50 μM, 75 μM, 100 μM, 150 μM, or 200 μM) |
| | Ammonium lactate (12%) |
| 529 | Suberin (3%) |
| | Arvelexin (10 μM, 25 μM, 50 μM, 75 μM, 100 μM, 150 μM, or 200 μM) |
| | Ammonium lactate (15%) |
| 530 | Suberin (3%) |
| | Arvelexin (10 μM, 25 μM, 50 μM, 75 μM, 100 μM, 150 μM, or 200 μM) |
| | Ammonium lactate (17.5%) |
| 531 | Suberin (3%) |
| | Arvelexin (10 μM, 25 μM, 50 μM, 75 μM, 100 μM, 150 μM, or 200 μM) |
| | Ammonium lactate (20%) |
| 532 | Cutin (1%-5%) |
| | Arvelexin (10 μM-200 μM) |
| | Ammonium lactate (12%-20%) |
| 533 | Cutin (1%, 2%, 3%, 4%, or 5%) |
| | Arvelexin (10 μM, 25 μM, 50 μM, 75 μM, 100 μM, 150 μM, or 200 μM) |
| | Ammonium lactate (12%) |
| 534 | Cutin (1%, 2%, 3%, 4%, or 5%) |
| | Arvelexin (10 μM, 25 μM, 50 μM, 75 μM, 100 μM, 150 μM, or 200 μM) |
| | Ammonium lactate (15%) |
| 535 | Cutin (1%, 2%, 3%, 4%, or 5%) |
| | Arvelexin (10 μM, 25 μM, 50 μM, 75 μM, 100 μM, 150 μM, or 200 μM) |
| | Ammonium lactate (17.5%) |
| 536 | Cutin (1%, 2%, 3%, 4%, or 5%) |
| | Arvelexin (10 μM, 25 μM, 50 μM, 75 μM, 100 μM, 150 μM, or 200 μM) |
| | Ammonium lactate (20%) |
| 537 | Lignin (1%-30%) |
| | Ammonium lactate (12%-20%) |
| 538 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Ammonium lactate (12%) |
| 539 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Ammonium lactate (15%) |
| 540 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Ammonium lactate (17.5%) |
| 541 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Ammonium lactate (20%) |
| 542 | Lignin (1%-30%) |
| | Astaxanthin (1%-10%) |
| | Ammonium lactate (12%-20%) |
| 543 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Ammonium lactate (12%) |
| 544 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Ammonium lactate (15%) |
| 545 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Ammonium lactate (17.5%) |
| 546 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Ammonium lactate (20%) |
| 547 | Lignin (1%-30%) |
| | Arvelexin (10 μM, 25 μM, 50 μM, 75 μM, 100 μM, 150 μM, or 200 μM) |
| | Ammonium lactate (12%-20%) |
| 548 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Ammonium lactate (12%) |
| 549 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Ammonium lactate (15%) |
| 550 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Ammonium lactate (17.5%) |
| 551 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Ammonium lactate (20%) |

Urea

Urea can be used in concentrations approved for over the counter use up to 20%, and by prescription use above 20%.

TABLE 9
Formulations for exfoliating or keratolytic topical compositions. Each
formulation below includes urea and at least one other primary agent.
The formulations below may be used in conjunction with or further include
one or more of the additional supplemental agents disclosed above.

| Formulation No. | Ingredients (% w/w or w/v, or as indicated) |
|---|---|
| 552 | Suberin (3%) |
| | Urea (1%-60%) |
| 553 | Suberin (3%) |
| | Urea (1%-20%) |
| 554 | Suberin (3%) |
| | Urea (30%) |
| 555 | Suberin (3%) |
| | Urea (40%) |
| 556 | Suberin (3%) |
| | Urea (50%) |
| 557 | Suberin (3%) |
| | Urea (60%) |
| 558 | Cutin (1%-5%) |
| | Urea (1%-60%) |
| 559 | Cutin (1%, 2%, 3%, 4%, or 5%) |
| | Urea (1%-20%) |
| 560 | Cutin (1%, 2%, 3%, 4%, or 5%) |
| | Urea (30%) |
| 561 | Cutin (1%, 2%, 3%, 4%, or 5%) |
| | Urea (40%) |
| 562 | Cutin (1%, 2%, 3%, 4%, or 5%) |
| | Urea (50%) |
| 563 | Cutin (1%, 2%, 3%, 4%, or 5%) |
| | Urea (60%) |
| 564 | Suberin (3%) |
| | Astaxanthin (1%, 2%, 3%, 4%, 5%, 7.5%, or 10%) |
| | Urea (1%-60%) |
| 565 | Suberin (3%) |
| | Astaxanthin (1%, 2%, 3%, 4%, 5%, 7.5%, or 10%) |
| | Urea (1%-20%) |
| 566 | Suberin (3%) |
| | Astaxanthin (1%, 2%, 3%, 4%, 5%, 7.5%, or 10%) |
| | Urea (30%) |
| 567 | Suberin (3%) |
| | Astaxanthin (1%, 2%, 3%, 4%, 5%, 7.5%, or 10%) |
| | Urea (40%) |
| 568 | Suberin (3%) |
| | Astaxanthin (1%, 2%, 3%, 4%, 5%, 7.5%, or 10%) |
| | Urea (50%) |
| 569 | Suberin (3%) |
| | Astaxanthin (1%, 2%, 3%, 4%, 5%, 7.5%, or 10%) |
| | Urea (60%) |
| 570 | Cutin (1%-5%) |
| | Astaxanthin (1%-10%) |
| | Urea (1%-60%) |
| 571 | Cutin (1%, 2%, 3%, 4%, or 5%) |
| | Astaxanthin (1%, 2%, 3%, 4%, 5%, 7.5%, or 10%) |
| | Urea (1%-20%) |
| 572 | Cutin (1%, 2%, 3%, 4%, or 5%) |
| | Astaxanthin (1%, 2%, 3%, 4%, 5%, 7.5%, or 10%) |
| | Urea (30%) |
| 573 | Cutin (1%, 2%, 3%, 4%, or 5%) |
| | Astaxanthin (1%, 2%, 3%, 4%, 5%, 7.5%, or 10%) |
| | Urea (40%) |
| 574 | Cutin (1%, 2%, 3%, 4%, or 5%) |
| | Astaxanthin (1%, 2%, 3%, 4%, 5%, 7.5%, or 10%) |
| | Urea (50%) |
| 575 | Cutin (1%, 2%, 3%, 4%, or 5%) |
| | Astaxanthin (1%, 2%, 3%, 4%, 5%, 7.5%, or 10%) |
| | Urea (60%) |
| 576 | Suberin (3%) |
| | Arvelexin (10 μM-200 μM) |
| | Urea (1%-60%) |
| 577 | Suberin (3%) |
| | Arvelexin (10 μM, 25 μM, 50 μM, 75 μM, 100 μM, 150 μM, or 200 μM) |
| | Urea (1%-20%) |
| 578 | Suberin (3%) |
| | Arvelexin (10 μM, 25 μM, 50 μM, 75 μM, 100 μM, 150 μM, or 200 μM) |
| | Urea (30%) |
| 579 | Suberin (3%) |
| | Arvelexin (10 μM, 25 μM, 50 μM, 75 μM, 100 μM, 150 μM, or 200 μM) |
| | Urea (40%) |
| 580 | Suberin (3%) |
| | Arvelexin (10 μM, 25 μM, 50 μM, 75 μM, 100 μM, 150 μM, or 200 μM) |
| | Urea (50%) |
| 581 | Suberin (3%) |
| | Arvelexin (10 μM, 25 μM, 50 μM, 75 μM, 100 μM, 150 μM, or 200 μM) |
| | Urea (60%) |
| 582 | Cutin (1%-5%) |
| | Arvelexin (10 μM-200 μM) |
| | Urea (1%-60%) |
| 583 | Cutin (1%, 2%, 3%, 4%, or 5%) |
| | Arvelexin (10 μM, 25 μM, 50 μM, 75 μM, 100 μM, 150 μM, or 200 μM) |
| | Urea (1%-20%) |
| 584 | Cutin (1%, 2%, 3%, 4%, or 5%) |
| | Arvelexin (10 μM, 25 μM, 50 μM, 75 μM, 100 μM, 150 μM, or 200 μM) |
| | Urea (30%) |
| 585 | Cutin (1%, 2%, 3%, 4%, or 5%) |
| | Arvelexin (10 μM, 25 μM, 50 μM, 75 μM, 100 μM, 150 μM, or 200 μM) |
| | Urea (40%) |
| 586 | Cutin (1%, 2%, 3%, 4%, or 5%) |
| | Arvelexin (10 μM, 25 μM, 50 μM, 75 μM, 100 μM, 150 μM, or 200 μM) |
| | Urea (50%) |
| 587 | Cutin (1%, 2%, 3%, 4%, or 5%) |
| | Arvelexin (10 μM, 25 μM, 50 μM, 75 μM, 100 μM, 150 μM, or 200 μM) |
| | Urea (60%) |
| 588 | Lignin (1%-30%) |
| | Urea (1%-60%) |
| 589 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Urea (1%-20%) |
| 590 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Urea (30%) |
| 591 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Urea (40%) |
| 592 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Urea (50%) |
| 593 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Urea (60%) |
| 594 | Lignin (1%-30%) |
| | Astaxanthin (1%-10%) |
| | Urea (1%-60%) |
| 595 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Urea (1%-20%) |
| 596 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Urea (30%) |
| 597 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Urea (40%) |
| 598 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Urea (50%) |
| 599 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Urea (60%) |
| 600 | Lignin (1%-30%) |
| | Arvelexin (10 μM, 25 μM, 50 μM, 75 μM, 100 μM, 150 μM, or 200 μM) |
| | Urea (1%-60%) |
| 601 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Urea (1%-20%) |
| 602 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Urea (30%) |
| 603 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Urea (40%) |
| 604 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Urea (50%) |
| 605 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Urea (60%) |

### Example 5: Antimicrobial Topical Compositions

Tables 10-11 below include formulations for one or more primary agents used in an antimicrobial composition. Both lysozyme and nisin have antimicrobial and anti-fungal/yeast properties. When infection occurs, the invading organism(s) cause local tissue damage which can spread. The host tissues will then mount a local immune response which leads to initially localized or widespread inflammation, depending on the degree and severity and the virility of the infectious agent. The inflammatory response in turn causes tissue damage with various degrees of wound which at times may lead to cellulitis, ulceration, and tissue necrosis, requiring long term wound care. In addition, biopolymers (suberin, cutin, lignin) also have their own antimicrobial (antiviral, antifungal/yeast, and antibacterial) properties. When combined with lysozyme, the biopolymers (suberin, cutin, lignin) have a unique capability to provide a greater stability to lysozyme degradation, which provide a stronger, longer-lasting, synergistic and additive antimicrobial response.

In powder formulation, the concentration of lysozyme can range between 0.005%-99.995% (preferably between 0.05% and 99.95%) by weight, or about 1.0 μg or more per gram of the material being treated. In solution form, the concentration of lysozyme can be about 2.0 μg/ml, 10 μg/ml, 25 μg/ml, 50 μg/ml, 100 μg/ml, 125 μg/ml, 500 μg/ml, 1000 μg/ml, 10 mg/L. Other concentrations of lysozyme that may be used in accordance with the embodiments described herein may include about 5-2000 mg, 50-1000 mg, 10-200 mg/kg, 10-2000 mg/kg, 0.00-500 ppm.

Concentrations of nisin that may be used in accordance with the embodiments described herein may include about 1 IU/ml, 25 IU/ml, 50 IU/ml, 100 IU/ml, 200 IU/ml, 300 IU/ml, 500 IU/ml, 2000 IU/ml. Other concentrations of lysozyme that may be used in accordance with the embodiments described herein may include 0.0-500 IU/gram, 50000-1000000 IU, 500-5000 IU.

Both astaxanthin and arvelexin have strong anti-inflammatory and antioxidant and wound healing properties which could reduce the degree of tissue damage and would allow accelerated rate of repair. Additionally, both astaxanthin and arvelexin have anti-microbial properties of their own which will provide synergistic response as compared to when lysozyme/nisin is used alone or in combination.

Furthermore, biopolymers (suberin, cutin or lignin), also have their own anti-inflammatory and anti-microbial properties and are also known to help in wound healing and thus accelerating repair.

Thus, the combinations in Tables 10-11 below will provide super-potent broad-spectrum antimicrobial products with synergistic antimicrobial capabilities not only in prevention and/or control of infection but also in treatment of infections as well as reducing tissue damage and simultaneously providing accelerated repair and wound healing.

Table 10 below includes exemplary formulations for topical antimicrobial compositions that include lysozyme and/or nisin (with or without a chelating agent) and at least one additional primary agent. The topical antimicrobial compositions may be applied to a user's body for any purpose that may benefit from use of an antimicrobial, including for those indications described herein. Specific non-limiting examples of indications that may be treated using a retinoid-based topical composition are discussed below. Additional active and/or non-active supplemental agents may also be included in or used in conjunction with the formulations below in accordance with the embodiments described herein. The formulations may also include one or more vehicle ingredients to provide a suitable vehicle for topical administration including, but not limited to, those described above. The exfoliating or keratolytic topical compositions below may be in the form of a cleanser, shampoo, toner, moisturizer, balm, milk, scrub, peel, mask, or various emollient, lotion, cream, gel, hydrogel, ointment, solution, rinse, soap, lubricants, applicators, condom, foam, wipe, or other suitable vehicle. The compositions may be processed and manufactured according to conventional methods known in the art.

TABLE 10
Formulations for antimicrobial topical compositions that include lysozyme
and/or nisin and one or more additional primary agents. Each of the formulations
below may be provided with or without ethanol or isopropanol. Further, the
formulations below may be used in conjunction with or further include one
or more of the additional supplemental agents disclosed above.

| Formulation No. | Ingredients (% w/w or w/v, or as indicated) |
|---|---|
| 606 | Suberin (3%) or Lignin (1%-30%) |
| | Lysozyme (1.0 μg/ml-1000 μg/ml) |
| 607 | Suberin (3%) or Lignin (1%-30%) |
| | Lysozyme (1.0-2000 mg) |
| 608 | Suberin (3%) or Lignin (1%-30%) |
| | Lysozyme (10-2000 mg/kg) |
| 609 | Suberin (3%) or Lignin (1%-30%) |
| | Lysozyme (0.0-500 ppm) |
| 610 | Suberin (3%) or Lignin (1%-30%) |
| | Nisin (1.0 IU/ml-2000 IU/ml) |
| 611 | Suberin (3%) or Lignin (1%-30%) |
| | Nisin (1.0 IU/gm-500 IU/gm) |
| 612 | Suberin (3%) or Lignin (1%-30%) |
| | Nisin (1.0 IU-1,000,000 IU) |
| 613 | Cutin (1%-5%) |
| | Lysozyme (1.0 μg/ml-1000 μg/ml) |
| 614 | Cutin (1%-5%) |
| | Lysozyme (1.0-2000 mg) |
| 615 | Cutin (1%-5%) |
| | Lysozyme (10-2000 mg/kg) |
| 616 | Cutin (1%-5%) |
| | Lysozyme (0.0-500 ppm) |
| 617 | Cutin (1%-5%) |
| | Nisin (1.0 IU/ml-2000 IU/ml) |
| 618 | Cutin (1%-5%) |
| | Nisin (1.0 IU/gm-500 IU/gm) |
| 619 | Cutin (1%-5%) |
| | Nisin (1.0 IU-1,000,000 IU) |
| 620 | Suberin (3%) or Lignin (1%-30%) |
| | Astaxanthin (1%-10%) |
| | Lysozyme (1.0 μg/ml-1000 μg/ml) |
| 621 | Suberin (3%) or Lignin (1%-30%) |
| | Astaxanthin (1%-10%) |
| | Lysozyme (1.0-2000 mg) |
| 622 | Suberin (3%) or Lignin (1%-30%) |
| | Astaxanthin (1%-10%) |
| | Lysozyme (10-2000 mg/kg) |
| 623 | Suberin (3%) or Lignin (1%-30%) |
| | Astaxanthin (1%-10%) |
| | Lysozyme (0.0-500 ppm) |
| 624 | Suberin (3%) or Lignin (1%-30%) |
| | Astaxanthin (1%, 2%, 3%, 4%, 5%, 7.5%, 10%) |
| | Lysozyme (1.0 μg/ml-1000 μg/ml) |
| 625 | Suberin (3%) or Lignin (1%-30%) |
| | Astaxanthin (1%, 2%, 3%, 4%, 5%, 7.5%, 10%) |
| | Lysozyme (1.0-2000 mg) |
| 626 | Suberin (3%) or Lignin (1%-30%) |
| | Astaxanthin (1%, 2%, 3%, 4%, 5%, 7.5%, 10%) |
| | Lysozyme (10-2000 mg/kg) |
| 627 | Suberin (3%) or Lignin (1%-30%) |
| | Astaxanthin (1%, 2%, 3%, 4%, 5%, 7.5%, 10%) |
| | Lysozyme (0.0-500 ppm) |
| 628 | Suberin (3%) or Lignin (1%-30%) |
| | Astaxanthin (1%-10%) |
| | Nisin (1.0 IU/ml-2000 IU/ml) |
| 629 | Suberin (3%) or Lignin (1%-30%) |
| | Astaxanthin (1%-10%) |
| | Nisin (1.0 IU/gm-500 IU/gm) |
| 630 | Suberin (3%) or Lignin (1%-30%) |
| | Astaxanthin (1%-10%) |
| | Nisin (1.0 IU-1,000,000 IU) |
| 631 | Suberin (3%) or Lignin (1%-30%) |
| | Astaxanthin (1%, 2%, 3%, 4%, 5%, 7.5%, 10%) |
| | Nisin (1.0 IU/ml-2000 IU/ml) |
| 632 | Suberin (3%) or Lignin (1%-30%) |
| | Astaxanthin (1%, 2%, 3%, 4%, 5%, 7.5%, 10%) |
| | Nisin (1.0 IU/gm-500 IU/gm) |
| 633 | Suberin (3%) or Lignin (1%-30%) |
| | Astaxanthin (1%, 2%, 3%, 4%, 5%, 7.5%, 10%) |
| | Nisin (1.0 IU-1,000,000 IU) |
| 634 | Suberin (3%) or Lignin (1%-30%) |
| | Arvelexin (10 μM-200 μM) |
| | Lysozyme (1.0 μg/ml-1000 μg/ml) |
| 635 | Suberin (3%) or Lignin (1%-30%) |
| | Arvelexin (10 μM-200 μM) |
| | Lysozyme (1.0-2000 mg) |
| 636 | Suberin (3%) or Lignin (1%-30%)Arvelexin (10 μM-200 μM) |
| | Lysozyme (10-2000 mg/kg) |
| 637 | Suberin (3%) or Lignin (1%-30%) |
| | Arvelexin (10 μM-200 μM) |
| | Lysozyme (0.0-500 ppm) |
| 638 | Suberin (3%) or Lignin (1%-30%) |
| | Arvelexin (10 μM, 25 μM, 50 μM, 75 μM, 100 μM, 150 μM, 200 μM) |
| | Lysozyme (1.0 μg/ml-1000 μg/ml) |
| 639 | Suberin (3%) or Lignin (1%-30%) |
| | Arvelexin (10 μM, 25 μM, 50 μM, 75 μM, 100 μM, 150 μM, 200 μM) |
| | Lysozyme (1.0-2000 mg) |
| 640 | Suberin (3%) or Lignin (1%-30%) |
| | Arvelexin (10 μM, 25 μM, 50 μM, 75 μM, 100 μM, 150 μM, 200 μM) |
| | Lysozyme (10-2000 mg/kg) |
| 641 | Suberin (3%) or Lignin (1%-30%) |
| | Arvelexin (10 μM, 25 μM, 50 μM, 75 μM, 100 μM, 150 μM, 200 μM) |
| | Lysozyme (0.0-500 ppm) |
| 642 | Cutin (1%-5%) |
| | Astaxanthin (1%-10%) |
| | Lysozyme (1.0 μg/ml-1000 μg/ml) |
| 643 | Cutin (1%-5%) |
| | Astaxanthin (1%-10%) |
| | Lysozyme (1.0-2000 mg) |
| 644 | Cutin (1%-5%) |
| | Astaxanthin (1%-10%) |
| | Lysozyme (10-2000 mg/kg) |
| 645 | Cutin (1%-5%) |
| | Astaxanthin (1%-10%) |
| | Lysozyme (0.0-500 ppm) |
| 646 | Cutin (1%-5%) |
| | Astaxanthin (1%, 2%, 3%, 4%, 5%, 7.5%, 10%) |
| | Lysozyme (1.0 μg/ml-1000 μg/ml) |
| 647 | Cutin (1%-5%) |
| | Astaxanthin (1%, 2%, 3%, 4%, 5%, 7.5%, 10%) |
| | Lysozyme (1.0-2000 mg) |
| 648 | Cutin (1%-5%) |
| | Astaxanthin (1%, 2%, 3%, 4%, 5%, 7.5%, 10%) |
| | Lysozyme (10-2000 mg/kg) |
| 649 | Cutin (1%-5%) |
| | Astaxanthin (1%, 2%, 3%, 4%, 5%, 7.5%, 10%) |
| | Lysozyme (0.0-500 ppm) |
| 650 | Suberin (3%) or Lignin (1%-30%) |
| | Arvelexin (10 μM-200 μM) |
| | Nisin (1.0 IU/ml-2000 IU/ml) |
| 651 | Suberin (3%) or Lignin (1%-30%) |
| | Arvelexin (10 μM-200 μM) |
| | Nisin (1.0 IU/gm-500 IU/gm) |
| 652 | Suberin (3%) or Lignin (1%-30%) |
| | Arvelexin (10 μM-200 μM) |
| | Nisin (1.0 IU-1,000,000 IU) |
| 653 | Suberin (3%) or Lignin (1%-30%) |
| | Arvelexin (10 μM, 25 μM, 50 μM, 75 μM, 100 μM, 150 μM, 200 μM) |
| | Nisin (1.0 IU/ml-2000 IU/ml) |
| 654 | Suberin (3%) or Lignin (1%-30%) |
| | Arvelexin (10 μM, 25 μM, 50 μM, 75 μM, 100 μM, 150 μM, 200 μM) |
| | Nisin (1.0 IU/gm-500 IU/gm) |
| 655 | Suberin (3%) or Lignin (1%-30%) |
| | Arvelexin (10 μM, 25 μM, 50 μM, 75 μM, 100 μM, 150 μM, 200 μM) |
| | Nisin (1.0 IU-1,000,000 IU) |
| 656 | Cutin (1%-5%) |
| | Arvelexin (10 μM-200 μM) |
| | Lysozyme (1.0 μg/ml-1000 μg/ml) |
| 657 | Cutin (1%-5%) |
| | Arvelexin (10 μM-200 μM) |
| | Lysozyme (1.0-2000 mg) |
| 658 | Cutin (1%-5%) |
| | Arvelexin (10 μM-200 μM) |
| | Lysozyme (10-2000 mg/kg) |
| 659 | Cutin (1%-5%) |
| | Arvelexin (10 μM-200 μM) |
| | Lysozyme (0.0-500 ppm) |
| 660 | Cutin (1%-5%) |
| | Arvelexin (10 μM, 25 μM, 50 μM, 75 μM, 100 μM, 150 μM, 200 μM) |
| | Lysozyme (1.0 μg/ml-1000 μg/ml) |
| 661 | Cutin (1%-5%) |
| | Arvelexin (10 μM, 25 μM, 50 μM, 75 μM, 100 μM, 150 μM, 200 μM) |
| | Lysozyme (1.0-2000 mg) |
| 662 | Cutin (1%-5%) |
| | Arvelexin (10 μM, 25 μM, 50 μM, 75 μM, 100 μM, 150 μM, 200 μM) |
| | Lysozyme (10-2000 mg/kg) |
| 663 | Cutin (1%-5%) |
| | Arvelexin (10 μM, 25 μM, 50 μM, 75 μM, 100 μM, 150 μM, 200 μM) |
| | Lysozyme (0.0-500 ppm) |
| 664 | Suberin (3%) or Lignin (1%-30%) |
| | Nisin (1.0 IU/ml-2000 IU/ml) |
| | Lysozyme (1.0 μg/ml-1000 μg/ml) or (1.0-2000 mg) or (10-2000 |
| | mg/kg) or (0.0-500 ppm) |
| 665 | Suberin (3%) or Lignin (1%-30%) |
| | Nisin (1.0 IU/ml-2000 IU/ml) or (1.0 IU/gm-500 IU/gm) or |
| | (1.0 IU-1,000,000 IU) |
| | Lysozyme (1.0 μg/ml-1000 μg/ml) or (1.0-2000 mg) or |
| | (10-2000 mg/kg) or (0.0-500 ppm) |
| 666 | Cutin (1%-5%) |
| | Nisin (1.0 IU/ml-2000 IU/ml) or (1.0 IU/gm-500 IU/gm) or |
| | (1.0 IU-1,000,000 IU) |
| | Lysozyme (1.0 μg/ml-1000 μg/ml) or (1.0-2000 mg) or |
| | (10-2000 mg/kg) or (0.0-500 ppm) |
| 667 | Suberin (3%) or Lignin (1%-30%) |
| | Astaxanthin (1%-10%) |
| | Nisin (1.0 IU/ml-2000 IU/ml) or (1.0 IU/gm-500 IU/gm) or |
| | (1.0 IU-1,000,000 IU) |
| | Lysozyme (1.0 μg/ml-1000 μg/ml) or (1.0-2000 mg) or |
| | (10-2000 mg/kg) or (0.0-500 ppm) |
| 668 | Suberin (3%) or Lignin (1%-30%) |
| | Astaxanthin (1%, 2%, 3%, 4%, 5%, 7.5%, 10%) |
| | Nisin (1.0 IU/ml-2000 IU/ml) or (1.0 IU/gm-500 IU/gm) or |
| | (1.0 IU-1,000,000 IU) |
| | Lysozyme (1.0 μg/ml-1000 μg/ml) or (1.0-2000 mg) or |
| | (10-2000 mg/kg) or (0.0-500 ppm) |
| 669 | Cutin (1%-5%) |
| | Astaxanthin (1%-10%) |
| | Nisin (1.0 IU/ml-2000 IU/ml) or (1.0 IU/gm-500 IU/gm) or |
| | (1.0 IU-1,000,000 IU) |
| | Lysozyme (1.0 μg/ml-1000 μg/ml) or (1.0-2000 mg) or |
| | (10-2000 mg/kg) or (0.0-500 ppm) |
| 670 | Cutin (1%-5%) |
| | Astaxanthin (1%, 2%, 3%, 4%, 5%, 7.5%, 10%) |
| | Nisin (1.0 IU/ml-2000 IU/ml) or (1.0 IU/gm-500 IU/gm) or |
| | (1.0 IU-1,000,000 IU) |
| | Lysozyme (1.0 μg/ml-1000 μg/ml) or (1.0-2000 mg) or |
| | (10-2000 mg/kg) or (0.0-500 ppm) |

The formulations in Table 10 may also include one or more additional antimicrobial agent, such as N-alkyl ethylbenzyl dimethyl ammonium chloride (C12-C14) (at a concentrate of about 0.05%, 0.154%, 1.25% by weight or volume), benzalkonium chloride (at a concentrate of about 0.05%, 0.154%, 1.25% by weight or volume), or an alcohol-based ingredient such as ethanol or isopropanol (e.g., at a concentration of greater than 60% ethanol or greater than 70% isopropanol as recommended by the CDC for use with hand rub products in healthcare settings), each of which are currently used by individuals and in healthcare settings.

Table 11 below includes exemplary formulations for topical antimicrobial compositions that include lysozyme and/or nisin, at least one additional primary agent, and at least one additional supplemental antimicrobial agent. The topical antimicrobial compositions may be applied to a user's body for any purpose that may benefit from use of an antimicrobial, including for those indications described herein. Specific non-limiting examples of indications that may be treated using a retinoid-based topical composition are discussed below. Additional active and/or non-active supplemental agents may also be included in or used in conjunction with the formulations below in accordance with the embodiments described herein. The formulations may also include one or more vehicle ingredients to provide a suitable vehicle for topical administration including, but not limited to, those described above. The exfoliating or keratolytic topical compositions below may be in the form of a cleanser, toner, moisturizer, scrub, peel, mask, or various emollient, lotion, cream, gel, hydrogel, ointment, solution, shampoo, rinse, soap, foam, wipe, or other suitable vehicle. The compositions may be processed and manufactured according to conventional methods known in the art.

TABLE 11
Formulations for antimicrobial topical compositions that include lysozyme
and/or nisin, one or more additional primary agents, and one or more additional
supplemental antimicrobial agent (N-alkyl ethylbenzyl dimethyl ammonium
chloride (C12-C14), benzalkonium chloride, ethanol or isopropanol). The
formulations below may be used in conjunction with or further include one
or more of the additional supplemental agents disclosed above.

| Formulation No. | Ingredients (% w/w or w/v, or as indicated) |
|---|---|
| 671 | Suberin (3%) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 672 | Cutin (1%-5%) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 673 | Lignin (1%-30%) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 674 | Suberin (3%) |
| | Astaxanthin (1%, 2%, 3%, 4%, 5%, 7.5%, 10%) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 675 | Cutin (1%-5%) |
| | Astaxanthin (1%, 2%, 3%) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 676 | Suberin (3%) |
| | Arvelexin (10 μM, 25 μM, 50 μM, 75 μM, 100 μM, 150 μM, 200 μM) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 677 | Cutin (1%-5%) |
| | Astaxanthin (1%, 2%, 3%, 4%, 5%, 7.5%, 10%) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 678 | Lignin (1%-30%) |
| | Astaxanthin (1%, 2%, 3%, 4%, 5%, 7.5%, 10%) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 679 | Lignin (1%-30%) |
| | Arvelexin (10 μM, 25 μM, 50 μM, 75 μM, 100 μM, 150 μM, 200 μM) |
| | N-alkyl ethylbenzyl dimethyl ammonium chloride (C12-C14) (0.05%, |
| | 0.154%, 1.25%) |
| | Benzalkonium chloride (0.05%, 0.154%, 1.25%) |
| 680 | Suberin (3%) |
| | Benzalkonium chloride (0.05%, 0.154%, 1.25%) |
| 681 | Cutin (1%-5%) |
| | Benzalkonium chloride (0.05%, 0.154%, 1.25%) |
| 682 | Lignin (1%-30%) |
| | Benzalkonium chloride (0.05%, 0.154%, 1.25%) |
| 683 | Suberin (3%) |
| | Astaxanthin (1%, 2%, 3%, 4%, 5%, 7.5%, 10%) |
| | Benzalkonium chloride (0.05%, 0.154%, 1.25%) |
| 684 | Cutin (1%-5%) |
| | Astaxanthin (1%, 2%, 3%) |
| | Benzalkonium chloride (0.05%, 0.154%,1.25%) |
| 685 | Suberin (3%) |
| | Arvelexin (10 μM, 25 μM, 50 μM, 75 μM, 100 μM, 150 μM, 200 μM) |
| | Benzalkonium chloride (0.05%, 0.154%, 1.25%) |
| 686 | Cutin (1%-5%) |
| | Astaxanthin (1%, 2%, 3%, 4%, 5%, 7.5%, 10%) |
| | Benzalkonium chloride (0.05%, 0.154%, 1.25%) |
| 687 | Lignin (1%-30%) |
| | Astaxanthin (1%, 2%, 3%, 4%, 5%, 7.5%, 10%) |
| | Benzalkonium chloride (0.05%, 0.154%, 1.25%) |
| 688 | Lignin (1%-30%) |
| | Arvelexin (10 μM, 25 μM, 50 μM, 75 μM, 100 μM, 150 μM, 200 μM) |
| | Benzalkonium chloride (0.05%, 0.154%, 1.25%) |
| 689 | Suberin (3%) |
| | N-alkyl ethylbenzyl dimethyl ammonium chloride (C12-C14) (0.05%, |
| | 0.154%, 1.25%) |
| 690 | Cutin (1%-5%) |
| | N-alkyl ethylbenzyl dimethyl ammonium chloride (C12-C14) (0.05%, |
| | 0.154%, 1.25%) |
| 691 | Lignin (1%-30%) |
| | N-alkyl ethylbenzyl dimethyl ammonium chloride (C12-C14) (0.05%, |
| | 0.154%, 1.25%) |
| 692 | Suberin (3%) |
| | Astaxanthin (1%, 2%, 3%, 4%, 5%, 7.5%, 10%) |
| | N-alkyl ethylbenzyl dimethyl ammonium chloride (C12-C14) (0.05%, |
| | 0.154%, 1.25%) |
| 693 | Cutin (1%-5%) |
| | Astaxanthin (1%, 2%, 3%) |
| | N-alkyl ethylbenzyl dimethyl ammonium chloride (C12-C14) (0.05%, |
| | 0.154%, 1.25%) |
| 694 | Suberin (3%) |
| | Arvelexin (10 μM, 25 μM, 50 μM, 75 μM, 100 μM, 150 μM, 200 μM) |
| | N-alkyl ethylbenzyl dimethyl ammonium chloride (C12-C14) (0.05%, |
| | 0.154%, 1.25%) |
| 695 | Cutin (1%-5%) |
| | astaxanthin (1%, 2%, 3%, 4%, 5%, 7.5%, 10%) |
| | N-alkyl ethylbenzyl dimethyl ammonium chloride (C12-C14) (0.05%, |
| | 0.154%, 1.25%) |
| 696 | Lignin (1%-30%) |
| | Astaxanthin (1%, 2%, 3%, 4%, 5%, 7.5%, 10%) |
| | N-alkyl ethylbenzyl dimethyl ammonium chloride (C12-C14) (0.05%, |
| | 0.154%, 1.25%) |
| 697 | Lignin (1%-30%) |
| | Arvelexin (10 μM, 25 μM, 50 μM, 75 μM, 100 μM, 150 μM, 200 μM) |
| | N-alkyl ethylbenzyl dimethyl ammonium chloride (C12-C14) (0.05%, |
| | 0.154%, 1.25%) |
| 698 | Suberin (3%) |
| | Ethanol (60%-100%) |
| 699 | Cutin (1%-5%) |
| | Ethanol (60%-100%) |
| 700 | Lignin (1%-30%) |
| | Ethanol (60%-100%) |
| 701 | Suberin (3%) |
| | Astaxanthin (1%, 2%, 3%, 4%, 5%, 7.5%, 10%) |
| | Ethanol (60%-100%) |
| 702 | Cutin (1%-5%) |
| | Astaxanthin (1%, 2%, 3%) |
| | Ethanol (60%-100%) |
| 703 | Suberin (3%) |
| | Arvelexin (10 μM, 25 μM, 50 μM, 75 μM, 100 μM, 150 μM, 200 μM) |
| | Ethanol (60%-100%) |
| 704 | Cutin (1%-5%) |
| | Astaxanthin (1%, 2%, 3%, 4%, 5%, 7.5%, 10%) |
| | Ethanol (60%-100%) |
| 705 | Lignin (1%-30%) |
| | Astaxanthin (1%, 2%, 3%, 4%, 5%, 7.5%, 10%) |
| | Ethanol (60%-100%) |
| 706 | Lignin (1%-30%) |
| | Arvelexin (10 μM, 25 μM, 50 μM, 75 μM, 100 μM, 150 μM, 200 μM) |
| | Ethanol (60%-100%) |
| 707 | Suberin (3%) |
| | Isopropanol (70%-100%) |
| 708 | Cutin (1%-5%) |
| | Isopropanol (70%-100%) |
| 709 | Lignin (1%-30%) |
| | Isopropanol (70%-100%) |
| 710 | Suberin (3%) |
| | Astaxanthin (1%, 2%, 3%, 4%, 5%, 7.5%, 10%) |
| | Isopropanol (70%-100%) |
| 711 | Cutin (1%-5%) |
| | Astaxanthin (1%, 2%, 3%) |
| | Isopropanol (70%-100%) |
| 712 | Suberin (3%) |
| | Arvelexin (10 μM, 25 μM, 50 μM, 75 μM, 100 μM, 150 μM, 200 μM) |
| | Isopropanol (70%-100%) |
| 713 | Cutin (1%-5%) |
| | Astaxanthin (1%, 2%, 3%, 4%, 5%, 7.5%, 10%) |
| | Isopropanol (70%-100%) |
| 714 | Lignin (1%-30%) |
| | Astaxanthin (1%, 2%, 3%, 4%, 5%, 7.5%, 10%) |
| | Isopropanol (70%-100%) |
| 715 | Lignin (1%-30%) |
| | Arvelexin (10 μM, 25 μM, 50 μM, 75 μM, 100 μM, 150 μM, 200 μM) |
| | Isopropanol (70%-100%) |

Optionally, formulations 671-697 in Table 11 may also include an alcohol-based ingredient such as ethanol or isopropanol (e.g., at a concentration of greater than 60% ethanol or greater than 70% isopropanol as recommended by the CDC for use with hand rub products in healthcare settings).

The compositions in Tables 10-11 above can be used to control and/or prevent of certain infections in humans, animals, plants, fruits, vegetables, food packaging; and can also be used to control of microbial growths in feedstocks and industrial products. The compositions in Tables 10-11 can be used in various cleansing products and antiseptics, for decontamination of surfaces amenable to microbial growths, for use as preservatives, to prevent and control and/or treat skin and oral or mucosal infection and for treatment or control of bed sore, pressure sore, pressure-induced skin ulcer (with and without dressing such as xeroform), or on oral (lip and oral mucosa) and skin of genitalia (skin and the mucosal surface of anogenital area).

The compositions in Tables 10-11 can be used in the areas of dentistry, gynecology, ophthalmology, and proctology as well as in dermatological or surgical settings for prevention or treatment of infections including, but not limited to, oral/genital herpes, cold sores, herpes labialis, herpes gladiatorum, herpetic whitlow, herpetic vulvovaginitis, gingivitis, aphthous ulcer, herpetic/aphthous stomatitis, aphthous ulcer, canker sore, oral/genital pemphigus, pemphigus vulgaris, pemphigoid, bullous pemphigoid, Hailey-Hailey disease, lip infection, skin/oral/mucosal impetigo, bullous impetigo, MRSA (Methicillin-resistant Staphylococcus aureus), hidradenitis suppurativa, acne vulgaris, skin folliculitis (e.g., in the form of shaving gel/cream, after shaving products, lotions), scalp folliculitis (e.g., in the form of shampoo, solution, gel, lip balm, milk), burned skin or mucosal surface, trauma-induced skin or mucosal tissues.

The compositions in Tables 10-11 can be used in a variety of wound dressing and wound debridement, wet wound dressing, in the form of lip cream, emollients, lipsticks, lip balm, lip gel, hydrogel, lip gloss, spray, solution, various forms of dental products, devices, removable dental products, film-forming solution, retainer, denture solution/containers/cleansers, etc. They may also contain natural/synthetic flavoring agents including but not limited to one or more of mint flavor, peach flavor, citrus, apple, cinnamon flavors, etc. or natural/synthetic fragrances. They can be used in the form of lubricants in different medical/surgical settings such as in gastroenterology, gynecological examination, and proctology. They can be used in various veterinary medical settings. They can also be used in any intimate or sexual contact as a means of protection and prevention of sexually transmitted infections or diseases in the forms of lubricant, wipe, gel, hydrogel, ointment, solution, rinse, soap, shampoo, lubricants, condom, emollients, lotion, spray, cleanser, wash before and after any contact with sex organ and/or other bodily parts that comes in contact with the individual and partner or any tools used in the process (as a means of disinfectant, decontamination, prevention). The compositions in Tables 10-11 can also be used in sponge, condom, and/or other contraceptive products or devices.

The compositions in Tables 10-11 can also be used in various forms of contact sports activities, such as boxing, basketball, wrestling, sumo wrestling, to prevent skin infections or as surface decontamination in any sports activity, gymnasium, clinics, urgent care facilities/hospital settings, medical, dental, or surgical units, veterinary medical settings, in-patient, out-patient setting, restaurants, home or public utilities/rest rooms, toilets, bathrooms, showers, bathtubs, etc. They can also be used as personal care, cleansers, soap, shower gel, body oil, liquids soap, bath products, lip balm, milk, face cleanser, hair products, make up removal, shampoo, conditioning, body wipes, moisturizers, antiseptics, lubricants with antibacterial, pre and post operation antiseptics, hand sanitizer, hand washing, dish washing/cleansers. The compositions in Tables 10-11 can also be used in an oral mouth wash/toothpaste (see, e.g., examples below). Because many skin/oral/mucosal infections are often tender, a local anesthetic (e.g., lidocaine, bupivacaine, mepivacaine, tetracaine, etc.) at a concentration range of about 1%-5% or as known in the art) may be added to any of the compositions described above to alleviate the pain and discomfort associated with eating, drinking, swallowing, or urination, or any discomfort associated with invasive procedure. They can also be used as antimicrobial containing lubricants prior to any medical procedures to facilitated insertion of instruments with and without local anesthetics.

### Example 6: Formulations for Treatment or Prevention of Dermatologic Conditions

Anti-Aging Conditions

Anti-aging topical compositions are used to treat or prevent aging processes (e.g., preventing wrinkles or age spots), dry skin, damage caused by environmental conditions, or other conditions associated with aging as described herein. Exemplary formulations for anti-aging topical compositions include Formulations 1-90 (moisturizers), 91-407 (Retinoid-based compositions), and 408-605 (exfoliants) as disclosed and described in the corresponding working examples. Additional active and/or non-active supplemental agents such as Pterostilbene and its derivatives (trans-3,5-dimethoxy-4-hydroxystilbene) (01%-100%, 1 mg-1000 mg, 1.0 μM-500 μM, 1.0 μg/mg-1000 μg/mL), Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone) may also be included in or used in conjunction with said formulations in accordance with the embodiments described herein.

The anti-aging formulations may also include one or more vehicle ingredients to provide a suitable vehicle for topical administration including, but not limited to, those described above. The anti-aging topical compositions may be in the form of a cleanser, toner, moisturizer, scrub, peel, mask, or various emollient, lotion, cream, gel, hydrogel, ointment, solution, ointment, shampoo, rinse, foam, balm, milk, wipe, or other suitable vehicle. The compositions may be processed and manufactured according to conventional methods known in the art.

Hyperkeratosis Conditions

Hyperkeratosis conditions clinically present with dry, flaky skin, and are usually accompanied with various degrees of inflammation and are typically itchy. They are usually treated with different topical medications such as retinoids, keratolytic agents such as urea, ammonium lactate, salicylic acid, various Alpha Hydroxy Acids/Beta Hydroxy Acids, and simultaneously treated with various moisturizers. Exemplary formulations for treatment of hyperkeratosis conditions include Formulations 408-605 as disclosed and described in the corresponding working examples. Additional active and/or non-active supplemental agents may also be included in or used in conjunction with said formulations in accordance with the embodiments described herein.

The formulations may also include one or more vehicle ingredients to provide a suitable vehicle for topical administration including, but not limited to, those described above. The topical compositions may be in the form of a cleanser, toner, moisturizer, scrub, peel, mask, or various emollient, lotion, cream, gel, hydrogel, ointment, rinse, solution, shampoo, soap, foam, balm, milk, wipe, or other suitable vehicle. The compositions may be processed and manufactured according to conventional methods known in the art.

Retinoid Dermatitis (and Other Sensitivity to Retinoids)

Retinoids are used extensively in various dermatological conditions such as acne and are widely used as potent anti-aging chemicals. Retinoid dermatitis is the most common side effect associated with the use of Retinoids. Retinoid dermatitis is characterized by pruritus (itch), dry skin, burning, irritation, peeling, redness, sensitivity to sun exposure (photosensitization) often requires the user to avoid excess sun exposure and taking extra precautionary measures for sun protection. Retinoid dermatitis is an inflammatory reaction and hence it is mediated through inflammatory pathways. Retinoid sensitivity is very common and can even develop with the lowest concentration of retinoids which often leads to discontinuation of retinoids or to reduce frequency of application which limits their use. Typically, it is treated with topical steroids. Because of their side effects, there have been many suggestions to relieve the symptoms associated with the use of retinoids, without much success.

Astaxanthin is a natural product with potent anti-inflammatory, antioxidant, and anti-microbial, and wound healing properties. In addition, astaxanthin and arvelexin possess intrinsic capability to block UV-radiation and to reverse the harmful effects of UV-exposure. Astaxanthin (and arvelexin) with their strong anti-inflammatory properties, if combined with retinoids, will effectively alleviate retinoid dermatitis which will enhance compliance. Furthermore, the intrinsic anti-UV effects of Astaxanthin, will provide additional protection against photosensitization commonly associated with the use of retinoids. Because of the strong antioxidant capabilities of astaxanthin and arvelexin, an unexpectedly super-potent anti-aging activity may be observed when added to retinoids (as compared to when retinoids used alone) which is better tolerated with enhanced compliance. Furthermore, addition of biopolymers with their multifunctional properties discussed herein will provide far superior anti-aging compositions.

Exemplary formulations for topical compositions to relieve the symptoms associated with the use of retinoids include Formulations 91-407 as disclosed and described in the corresponding working examples. Additional active and/or non-active supplemental agents may also be included in or used in conjunction with said formulations in accordance with the embodiments described herein.

The formulations may also include one or more vehicle ingredients to provide a suitable vehicle for topical administration including, but not limited to, those described above. The topical compositions may be in the form of a cleanser, toner, moisturizer, scrub, peel, mask, or various emollient, lotion, cream, gel, hydrogel, ointment, solution, shampoo, foam, wipe, or other suitable vehicle. The compositions may be processed and manufactured according to conventional methods known in the art.

Acne

Exemplary formulations for topical compositions to treat or prevent acne include retinoid-based compositions including Formulations 91-407 as disclosed and described in the corresponding working examples and beta-hydroxy acid-based compositions including Formulations 453-506 as disclosed and described in the corresponding working examples. Additional active and/or non-active supplemental agents such as Pterostilbene and its derivatives (trans-3,5-dimethoxy-4-hydroxystilbene) (01%-100%, 1 mg-1000 mg, 1.0 μM-500 μM, 1.0 μg/mg-1000 μg/mL). Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone) may also be included in or used in conjunction with said formulations in accordance with the embodiments described herein.

In addition, treatment or prevention of acne may also include compositions with azelaic acid, in accordance with Table 12 below

TABLE 12
Formulations for anti-acne compositions that include azelaic acid. The
formulations below may be used in conjunction with or further include
one or more of the additional supplemental agents disclosed above. Additionally,
Azelaic Acid (at concentrations specified herein) may be used alone or
in combination with one or more of supplemental agents.

| Formulation No. | Ingredients (% w/w or w/v, or as indicated) |
|---|---|
| 716 | Suberin (3%) |
| | Azelaic Acid (1%-20%) |
| 717 | Suberin (3%) |
| | Azelaic Acid (5%) |
| 718 | Suberin (3%) |
| | Azelaic Acid (10%) |
| 719 | Suberin (3%) |
| | Azelaic Acid (15%) |
| 720 | Suberin (3%) |
| | Azelaic Acid (20%) |
| 721 | Cutin (1%-5%) |
| | Azelaic Acid (1%-20%) |
| 722 | Cutin (1%, 2%, 3%, 4%, or 5%) |
| | Azelaic Acid (5%) |
| 723 | Cutin (1%, 2%, 3%, 4%, or 5%) |
| | Azelaic Acid (10%) |
| 724 | Cutin (1%, 2%, 3%, 4%, or 5%) |
| | Azelaic Acid (15%) |
| 725 | Cutin (1%, 2%, 3%, 4%, or 5%) |
| | Azelaic Acid (20%) |
| 726 | Suberin (3%) |
| | Astaxanthin (1%, 2%, 3%, 4%, 5%, 7.5%, or 10%) |
| | Azelaic Acid (1%-20%) |
| 727 | Suberin (3%) |
| | Astaxanthin (1%, 2%, 3%, 4%, 5%, 7.5%, or 10%) |
| | Azelaic Acid (5%) |
| 728 | Suberin (3%) |
| | Astaxanthin (1%, 2%, 3%, 4%, 5%, 7.5%, or 10%) |
| | Azelaic Acid (10%) |
| 729 | Suberin (3%) |
| | Astaxanthin (1%, 2%, 3%, 4%, 5%, 7.5%, or 10%) |
| | Azelaic Acid (15%) |
| 730 | Suberin (3%) |
| | Astaxanthin (1%, 2%, 3%, 4%, 5%, 7.5%, or 10%) |
| | Azelaic Acid (20%) |
| 731 | Cutin (1%-5%) |
| | Astaxanthin (1%-10%) |
| | Azelaic Acid (1%-20%) |
| 732 | Cutin (1%, 2%, 3%, 4%, or 5%) |
| | Astaxanthin (1%, 2%, 3%, 4%, 5%, 7.5%, or 10%) |
| | Azelaic Acid (5%) |
| 733 | Cutin (1%, 2%, 3%, 4%, or 5%) |
| | Astaxanthin (1%, 2%, 3%, 4%, 5%, 7.5%, or 10%) |
| | Azelaic Acid (10%) |
| 734 | Cutin (1%, 2%, 3%, 4%, or 5%) |
| | Astaxanthin (1%, 2%, 3%, 4%, 5%, 7.5%, or 10%) |
| | Azelaic Acid (15%) |
| 735 | Cutin (1%, 2%, 3%, 4%, or 5%) |
| | Astaxanthin (1%, 2%, 3%, 4%, 5%, 7.5%, or 10%) |
| | Azelaic Acid (20%) |
| 736 | Suberin (3%) |
| | Arvelexin (10 μM-200 μM) |
| | Azelaic Acid (1%-20%) |
| 737 | Suberin (3%) |
| | Arvelexin (10 μM, 25 μM, 50 μM, 75 μM, 100 μM, 150 μM, or 200 μM) |
| | Azelaic Acid (5%) |
| 738 | Suberin (3%) |
| | Arvelexin (10 μM, 25 μM, 50 μM, 75 μM, 100 μM, 150 μM, or 200 μM) |
| | Azelaic Acid (10%) |
| 739 | Suberin (3%) |
| | Arvelexin (10 μM, 25 μM, 50 μM, 75 μM, 100 μM, 150 μM, or 200 μM) |
| | Azelaic Acid (15%) |
| 740 | Suberin (3%) |
| | Arvelexin (10 μM, 25 μM, 50 μM, 75 μM, 100 μM, 150 μM, or 200 μM) |
| | Azelaic Acid (20%) |
| 741 | Cutin (1%-5%) |
| | Arvelexin (10 μM-200 μM) |
| | Azelaic Acid (1%-20%) |
| 742 | Cutin (1%, 2%, 3%, 4%, or 5%) |
| | Arvelexin (10 μM, 25 μM, 50 μM, 75 μM, 100 μM, 150 μM, or 200 μM) |
| | Azelaic Acid (5%) |
| 743 | Cutin (1%, 2%, 3%, 4%, or 5%) |
| | Arvelexin (10 μM, 25 μM, 50 μM, 75 μM, 100 μM, 150 μM, or 200 μM) |
| | Azelaic Acid (10%) |
| 744 | Cutin (1%, 2%, 3%, 4%, or 5%) |
| | Arvelexin (10 μM, 25 μM, 50 μM, 75 μM, 100 μM, 150 μM, or 200 μM) |
| | Azelaic Acid (15%) |
| 745 | Cutin (1%, 2%, 3%, 4%, or 5%) |
| | Arvelexin (10 μM, 25 μM, 50 μM, 75 μM, 100 μM, 150 μM, or 200 μM) |
| | Azelaic Acid (20%) |
| 746 | Lignin (1%-30%) |
| | Azelaic Acid (1%-20%) |
| 747 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Azelaic Acid (5%) |
| 748 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Azelaic Acid (10%) |
| 749 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Azelaic Acid (15%) |
| 750 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Azelaic Acid (20%) |
| 751 | Lignin (1%-30%) |
| | Astaxanthin (1%-10%) |
| | Azelaic Acid (1%-20%) |
| 752 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Azelaic Acid (5%) |
| 753 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Azelaic Acid (10%) |
| 754 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Azelaic Acid (15%) |
| 755 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Astaxanthin (1%, 2%, 3%, 4%, 5%, 7.5%, or 10%) |
| | Azelaic Acid (20%) |
| 756 | Lignin (1%-30%) |
| | Arvelexin (10 μM, 25 μM, 50 μM, 75 μM, 100 μM, 150 μM, or 200 μM) |
| | Azelaic Acid (1%-20%) |
| 757 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Azelaic Acid (5%) |
| 758 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Azelaic Acid (10%) |
| 759 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Azelaic Acid (15%) |
| 760 | Lignin (1%, 2%, 3%, 4%, 5%, 6%, 7%, 8%, 9%, 10%, 11%, 12%, 13%, |
| | 14%, 15%, 16%, 17%, 18%, 19%, 20%, 21%, 22%, 23%, 24%, |
| | 25%, 26%, 27%, 28%, 29%, or 30%) |
| | Arvelexin (10 μM, 25 μM, 50 μM, 75 μM, 100 μM, 150 μM, or 200 μM) |
| | Azelaic Acid (20%) |

In addition, antimicrobial agents—including lysozyme and/or nisin—may be used for treatment or prevention of acne and folliculitis, and impetigo, hidradenitis suppurativa, MRSA (Methicillin resistant Staphylococcus aureus) in the forms of topical emollients and/or wash, cleansers, rinse, soap, wipe, foam, shaving gel/cream/foam, hydrogel, lotion, shampoo, and the like. Exemplary formulations for antimicrobial compositions to treat or prevent acne include Formulations 606-670 as disclosed and described in the corresponding working examples. These formulations may be used in conjunction with or further include one or more of the additional supplemental agents disclosed above.

The formulations may also include one or more vehicle ingredients to provide a suitable vehicle for topical administration including, but not limited to, those described above. The topical compositions may be in the form of a cleanser, toner, shampoo, moisturizer, scrub, peel, mask, or various emollient, lotion, cream, gel, hydrogel, ointment, solution, foam, wipe, or other suitable vehicle. The compositions may be processed and manufactured according to conventional methods known in the art.

Rosacea and Acne Rosacea (with and without Sunscreen)

Rosacea and acne rosacea are common inflammatory skin conditions manifested by intense erythema of skin on nose, cheeks, chin, and forehead. The etiology of rosacea has been associated with the mite species Demodex folliculorum which resides inside the pilosebaceous unit which surrounds the hair follicles of face. The role of bacterial causes including Bacillus oleronius and Staphylococcus epidermidis have also been implicated. (Microbiology, 2012 DOI: 10.1099/jmm.0.048090-0). Aggravating factors of rosacea include UV exposure.

Both astaxanthin and arvelexin have antimicrobial and anti-inflammatory and antioxidant properties and both are known to provide UV protection. Similarly, biopolymers (suberin, and/or cutin, or lignin) are known to have antimicrobial and anti-inflammatory and antioxidant properties, and also provide protection against UV-exposure. Each of these compounds (astaxanthin, arvelexin or biopolymers, either alone or in combination) can be used for prevention, delay or treatment of rosacea and acne rosacea.

Rosacea and acne rosacea are typically treated by various topical or oral medications such as Azelaic acid (5%, 7%, 10%, 15%, 20% in the form of cream, lotion, foam, gel, hydrogel, ointment, solution, etc.) or metronidazole (0.05%, 0.75%, 1%) or sodium sulfacetamide and sulfur (1%, 5%, 10%), retinoids (various concentrations), topical minocycline (1%, 1.5%, 3% in the form of gel, foam, cream, etc.), topical Stromectol 1% (trade name ivermectin gel, cream) and oral ivermectin 3.0 mg. Topical combination of astaxanthin, or arvelexin, or plant-derived biopolymers (suberin, cutin, or lignin) and any of the following compounds including, but not limited to, azelaic acid, sodium sulfacetamide, metronidazole, ivermectin, Oramectin, Ivermectin, Eprinomectin, Eprimectin, Avermectin, Moxidectin. Abamectin—all with and without sun screen—as well as oral combination of astaxanthin (8 mg, 12, mg, 24 mg) with Stromectol 3 mg, trade name ivermectin (3 mg) or oral metronidaole (trade name Flagyl, 400 mg, or 500 mg) or Oramectin. Eprinomectin, Eprimectin, Avermectin, Moxidectin, Abamectin, can also be used effectively which will provide synergistic, and added response and will provide significant advantages over the currently available treatment options for rosacea and acne rosacea.

Table 13 below provides exemplary formulations for topical compositions that can be used to treat rosacea and/or acne rosacea. Additional active and/or non-active supplemental agents may also be included in or used in conjunction with the formulations below in accordance with the embodiments described herein. The formulations may also include one or more vehicle ingredients to provide a suitable vehicle for topical administration including, but not limited to, those discussed above. The compositions may be processed and manufactured according to conventional methods known in the art.

TABLE 13
Formulations of compositions for treating rosacea and/or acne rosacea.
The formulations below may be used in conjunction with or further include
one or more of the additional supplemental agents disclosed above. Additionally,
Azelaic Acid (at concentrations specified herein) may be used alone or
in combination with one or more of supplemental agents.

| Formulation No. | Ingredients (% w/w or w/v, or as indicated) |
|---|---|
| 761 | Suberin (3%) |
| | Azelaic Acid (5%, 10%, 15%, 20%) |
| 762 | Suberin (3%) |
| | Ivermectin (1%), or each of the following (Oramectin, Eprinomectin, |
| | Eprimectin, Avermectin, Moxidectin, Abamectin) |
| 763 | Suberin (3%) |
| | Metronidazole (0.05%, 0.75%, 1%) |
| 764 | Cutin (1%-5.0%) |
| | Azelaic Acid (5%, 10%, 15%, 20%) |
| 765 | Cutin (1%-5%) |
| | Ivermectin (1%) or each of the following (Oramectin, Eprinomectin, |
| | Eprimectin, Avermectin, Moxidectin, Abamectin) |
| 766 | Cutin (1%-5%) |
| | Metronidazole (0.05%, 0.75%, 1%) |
| 767 | Astaxanthin (1%, 2%, 3%, 4%, 5%, 7.5%, 10%) |
| | Azelaic Acid (5%, 10%, 15%, 20%) |
| 768 | Astaxanthin (1%, 2%, 3%, 4%, 5%, 7.5%, 10%) |
| | Metronidazole (0.05%, 0.75%, 1%) |
| 769 | Astaxanthin (1%, 2%, 3%) |
| | Ivermectin (1%) or each of the following (Oramectin, Eprinomectin, |
| | Eprimectin, Avermectin, Moxidectin, Abamectin) |
| 770 | Suberin (3%) |
| | Astaxanthin (1%, 2%, 3%, 4%, 5%, 7.5%, 10%) |
| | Azelaic Acid (5%, 10%, 15%, 20%) |
| 771 | Suberin (3%) |
| | Astaxanthin (1%, 2%, 3%, 4%, 5%, 7.5%, 10%) |
| | Ivermectin (1%) or each of the following (Oramectin, Eprinomectin, |
| | Eprimectin, Avermectin, Moxidectin, Abamectin) |
| 772 | Suberin (3%) |
| | Astaxanthin (1%, 2%, 3%, 4%, 5%, 7.5%, 10%) |
| | Metronidazole (0.05%, 0.75%, 1%) |
| 773 | Suberin (3%) |
| | Astaxanthin (1%, 2%, 3%, 4%, 5%, 7.5%, 10%) |
| | Ivermectin (1%) or each of the following (Oramectin, Eprinomectin, |
| | Eprimectin, Avermectin, Moxidectin, Abamectin) |
| 774 | Cutin (1%-5%) |
| | Astaxanthin (1%, 2%, 3%, 4%, 5%, 7.5%, 10%) |
| | Azelaic Acid (5%, 10%, 15%, 20%) |
| 775 | Cutin (1%-5%) |
| | Astaxanthin (1%, 2%, 3%, 4%, 5%, 7.5%,10%) |
| | Ivermectin (1%) or each of the following (Oramectin, Eprinomectin, |
| | Eprimectin, Avermectin, Moxidectin, Abamectin) |
| 776 | Cutin (1%-5%) |
| | Astaxanthin (1%, 2%, 3%, 4%, 5%, 7.5%, 10%) |
| | Metronidazole (0.05%, 0.75%, 1%) |
| 777 | Arvelexin (10 μM, 25 μM, 50 μM, 75 μM, 100 μM, 150 μM, or 200 μM) |
| | Azelaic Acid (5%, 10%, 15%, 20%) |
| 778 | Arvelexin (10 μM, 25 μM, 50 μM, 75 μM, 100 μM, 150 μM, or 200 μM) |
| | Ivermectin (1%) or each of the following (Oramectin, Eprinomectin, |
| | Eprimectin, Avermectin, Moxidectin, Abamectin) |
| 779 | Arvelexin (10 μM, 25 μM, 50 μM, 75 μM, 100 μM, 150 μM, or 200 μM) |
| | Metronidazole (0.05%, 0.75%, 1% |
| 780 | Suberin (3%) |
| | Arvelexin (10 μM, 25 μM, 50 μM, 75 μM, 100 μM, 150 μM, or 200 μM) |
| | Azelaic Acid (5%, 10%, 15%, 20%) |
| 781 | Suberin (3%) |
| | Arvelexin (10 μM, 25 μM, 50 μM, 75 μM, 100 μM, 150 μM, or 200 μM) |
| | Metronidazole (0.05%, 0.75%, 1%) |
| 782 | Suberin (3%) |
| | Arvelexin (10 μM, 25 μM, 50 μM, 75 μM, 100 μM, 150 μM, or 200 μM) |
| | Ivermectin (1%) or each of the following (Oramectin, Eprinomectin, |
| | Eprimectin, Avermectin, Moxidectin, Abamectin) |
| 783 | Cutin (1%-5%) |
| | Arvelexin (10 μM, 25 μM, 50 μM, 75 μM, 100 μM, 150 μM, or 200 μM) |
| | Ivermectin (1%) or each of the following (Oramectin, Eprinomectin, |
| | Eprimectin, Avermectin, Moxidectin, Abamectin) |
| 784 | Astaxanthin (1%, 2%, 3%, 4%, 5%, 7.5%, 10%) |
| | Arvelexin (10 μM, 25 μM, 50 μM, 75 μM, 100 μM, 150 μM, or 200 μM) |
| | Azelaic Acid (5%, 10%, 15%, 20%) |
| 785 | Astaxanthin (1%, 2%, 3%, 4%, 5%, 7.5%, 10%) |
| | Arvelexin (10 μM, 25 μM, 50 μM, 75 μM, 100 μM, 150 μM, or 200 μM) |
| | Metronidazole (0.05%, 0.75%, 1%) |
| 786 | Astaxanthin (1%, 2%, 3%, 4%, 5%, 7.5%, 10%) |
| | Arvelexin (10 μM, 25 μM, 50 μM, 75 μM, 100 μM, 150 μM, or 200 μM) |
| | Ivermectin (1%) or each of the following (Oramectin, Eprinomectin, |
| | Eprimectin, Avermectin, Moxidectin, Abamectin) |
| 787 | Lignin (1%-30%) |
| | Azelaic Acid (5%, 10%, 15%, 20%) |
| 788 | Lignin (1%-30%) |
| | Ivermectin (1%) or each of the following (Oramectin, Eprinomectin, |
| | Eprimectin, Avermectin, Moxidectin, Abamectin) |
| 789 | Lignin (1%-30%) |
| | Metronidazole (0.05%, 0.75%, 1%) |
| 790 | Lignin (1%-30%) |
| | Astaxanthin (1%, 2%, 3%, 4%, 5%, 7.5%, 10%) |
| | Azelaic Acid (5%, 10%, 15%, 20%) |
| 791 | Lignin (1%-30%) |
| | Astaxanthin (1%, 2%, 3%, 4%, 5%, 7.5%, 10%) |
| | Ivermectin (1%) or each of the following (Oramectin, Eprinomectin, |
| | Eprimectin, Avermectin, Moxidectin, Abamectin) |
| 792 | Lignin (1%-30%) |
| | Astaxanthin (1%, 2%, 3%, 4%, 5%, 7.5%, 10%) |
| | Azelaic Acid (5%, 10%, 15%, 20%) |
| 793 | Lignin (1%-30%) |
| | Astaxanthin (1%, 2%, 3%, 4%, 5%, 7.5%, 10%) and Stromectol 1% |
| 794 | Lignin (1%-30%) |
| | Astaxanthin (1%, 2%, 3%, 4%, 5%, 7.5%, 10%) |
| | Metronidazole (0.05%, 0.75%, 1%) |
| 795 | Lignin (1%-30%) |
| | Arvelexin (10 μM, 25 μM, 50 μM, 75 μM, 100 μM, 150 μM, or 200 μM) |
| | Azelaic Acid (5%, 10%, 15%, 20%) |
| 796 | Lignin (1%-30%) |
| | Arvelexin (10 μM, 25 μM, 50 μM, 75 μM, 100 μM, 150 μM, or 200 μM) |
| | Metronidazole (0.05%, 0.75%, 1%) |
| 797 | Lignin (1%-30%) |
| | Arvelexin (10 μM, 25 μM, 50 μM, 75 μM, 100 μM, 150 μM, or 200 μM) |
| | Ivermectin (1%) or each of the following (Oramectin, Eprinomectin, |
| | Eprimectin, Avermectin, Moxidectin, Abamectin) |

Additionally, astaxanthin, or arvelexin, and lysozyme, nisin, and plant-derived biopolymers (suberin, cutin, or lignin) in various combination can be used in the form of face wash, moisturizer, face cleanser, aftershave, balm, milk, or hair cleaner (e.g., lotion, foam, gel, liquid, bar, shampoo, etc.) to provide adjunct treatment for rosacea and acne rosacea. Exemplary adjunct treatment formulations that may be used in conjunction with the treatment of rosacea and acne rosacea include Formulations 1-90, 606-670 as disclosed and described in the corresponding working examples. These formulations may be used in conjunction with or further include one or more of the additional supplemental agents disclosed above.

Similarly, sunscreens in combination with supplemental agents such as pterostilbene and its derivatives (trans-3,5-dimethoxy-4-hydroxystilbene) (01%-100%, 1 mg-1000 mg, 1.0 μM-500 μM, 1.0 μg/mg-1000 μg/mL), or Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone) and/or containing astaxanthin, arvelexin, either alone or in combination with a plant-drive biopolymer (suberin, cutin, lignin) due to their superior antioxidant, anti-microbial, and anti-inflammatory properties and the fact that they possess their own UV light blocking properties, will provide advantageous over current sunscreens for rosacea and acne rosacea patients. Exemplary formulations for sunscreen compositions that may be used in conjunction with the treatment of rosacea and acne rosacea include Formulations 882-917 as disclosed and described in the corresponding working examples. These formulations may be used in conjunction with or further include one or more of the additional supplemental agents such as vitamin D, disclosed above.

Melasma/Hyperpigmentation

Hydroquinone is a bleaching cream (2% is available over the counter and 4% with prescription) that is widely used in many different OTC and prescription products for treatment of pigmentary skin conditions such as melasma, hyperpigmentation, scar-related pigmentation, acne-related pigmentation, dark circles under eyes, sun related pigmentary changes, freckles, solar lentigo, etc.

Melasma and other hyperpigmentation conditions are often treated with hydroquinone (a bleaching agent) in combination with other agents such as retinoids and topical steroids to improve effectiveness and to reduce side effects. For example, Tri-Luma® cream (which is a combination of tretinoin 0.05%+hydroquinone 4%+fluocinonide acetonide 0.01%) is FDA approved for treatment of melasma and many dermatologists also use it off-label for short term treatment of other hyperpigmented skin conditions (for a 6-8-week duration). The reason for adding fluocinonide (topical steroid) is to reduce the discomfort and to increase tolerability of retinoid sensitivity (i.e., retinoid dermatitis).

The problem with Tri-Luma® cream is that topical steroid fluocinonide 0.01% cream can only be used for a limited period of 6-8 weeks, because overuse of topical steroids can cause skin atrophy. To address these drawbacks, formulations having a combination of retinoids with (i) plant-derived biopolymers and/or (ii) astaxanthin, and/or (iii) arvelexin, and/or (iv) hydroquinone (at approximately 4%±2%) will provide a great alternative to Tri-Luma® cream, for the treatment of melasma/hyperpigmentation, for many reasons.

First, the anti-inflammatory properties of astaxanthin, arvelexin, and biopolymers will provide an opportunity to eliminate the need for using topical steroids (fluocinonide), and some supplemental agents such as pterostilbene and its derivatives (trans-3,5-dimethoxy-4-hydroxystilbene) (01%-100%, 1 mg-1000 mg, 1.0 μM-500 μM, 1.0 μg/mg-1000 μg/mL), or Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone), Betanin, Betalain, Betacyanins, Betaxanthins, making it much safer for long term use (i.e. steroid spearing effect).

Second, both plant-derived biopolymers and astaxanthin and some supplemental agents such as pterostilbene and equol and lignin peroxidase have their own skin whitening capabilities which provide a synergistic response in treatment of melasma and other hyperpigmentation conditions.

Further, the antioxidant properties of plant-derived biopolymers, astaxanthin, and arvelexin will also provide stronger antiaging products which can also reverse the UV radiation induced pathology.

Table 14 below include exemplary formulations for topical compositions for the treatment of melasma, freckles and other pigmentary skin conditions. Additional active and/or non-active supplemental agents such as Pterostilbene or its derivatives (trans-3,5-dimethoxy-4-hydroxystilbene) (01%-100%, 1 mg-1000 mg, 1.0 M-500 μM, 1.0 μg/mg-1000 μg/mL), Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone), lignin peroxidase may also be included in or used in conjunction with said formulations in accordance with the embodiments described herein, may also be included in or used in conjunction with the formulations below in accordance with the embodiments described herein. The formulations may also include one or more vehicle ingredients to provide a suitable vehicle for topical administration including, but not limited to, those described above (e.g., cream, lotion, gel, hydrogel, ointment, solution, foam, balm, milk, etc.). The compositions may be processed and manufactured according to conventional methods known in the art.

TABLE 14
Formulations of compositions for treating melasma, freckles and other pigmentary skin
conditions. The formulations below may be used in conjunction with or further include
one or more of the additional supplemental agents disclosed above. Additionally, the
retinoid (at the concentrations specified herein) may be used alone or in combination
with one or more supplemental agents for treatment of such pigmentary skin conditions.

| Formulation No. | Ingredients (% w/w or w/v, or as indicated) |
|---|---|
| 799 | Suberin (3%) |
| | Tretinoin (0.025%-1%, 0.025%-0.05%, 0.05%-0.075%, 0.075%-0.1%, |
| | 0.1%-1.0%) |
| 800 | Suberin (3%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 801 | Suberin (3%) |
| | Retinol (0.5%-3%, 0.5%, 1.0%, 1.5%, 2.0%, 2.5%, 3%) |
| 802 | Suberin (3%) |
| | Retinol (0.5%-3%, 0.5%, 1.0%, 1.5%, 2.0%, 2.5%, 3%) |
| | Hydroquinone (2%-4%) |
| 803 | Cutin (1%-5%) |
| | Tretinoin (0.025%-1%, 0.025%-0.05%, 0.05%-0.075%, 0.075%-0.1%, |
| | 0.1%-1.0%) |
| 804 | Cutin (1%-5%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 805 | Cutin (1%-5%) |
| | Retinol (0.5%-3%, 0.5%, 1.0%, 1.5%, 2.0%, 2.5%, 3%) |
| 806 | Cutin (1%-5%) |
| | Retinol (0.5%-3%, 0.5%, 1.0%, 1.5%, 2.0%, 2.5%, 3%) |
| | Hydroquinone (2%-4%) |
| 807 | Astaxanthin (1%-10%) |
| | Tretinoin (0.025%-1%, 0.025%-0.05%, 0.05%-0.075%, 0.075%-0.1%, |
| | 0.1%-1.0%) |
| 808 | Astaxanthin (1%-10%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 809 | Astaxanthin (1%, 2%, 3%, 4%, 5%, 7.5%, or 10%) |
| | Tretinoin (0.025%-1%, 0.025%-0.05%, 0.05%-0.075%, 0.075%-0.1%, |
| | 0.1%-1.0%) |
| 810 | Astaxanthin (1%, 2%, 3%, 4%, 5%, 7.5%, or 10%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 811 | Astaxanthin (1%-10%) |
| | Retinol (0.5%-3%, 0.5%, 1.0%, 1.5%, 2.0%, 2.5%, 3%) |
| 812 | Astaxanthin (1%-10%) |
| | Retinol (0.5%-3%, 0.5%, 1.0%, 1.5%, 2.0%, 2.5%, 3%) |
| | Hydroquinone (2%-4%) |
| 813 | Astaxanthin (1%, 2%, 3%, 4%, 5%, 7.5%, or 10%) |
| | Retinol (0.5%-3%, 0.5%, 1.0%, 1.5%, 2.0%, 2.5%, 3%) |
| 814 | Astaxanthin (1%, 2%, 3%, 4%, 5%, 7.5%, or 10%) |
| | Retinol (0.5%-3%, 0.5%, 1.0%, 1.5%, 2.0%, 2.5%, 3%) |
| | Hydroquinone (2%-4%) |
| 815 | Suberin (3%) |
| | Astaxanthin (1%-10%) |
| | Tretinoin (0.025%-1%, 0.025%-0.05%, 0.05%-0.075%, 0.075%-0.1%, |
| | 0.1%-1.0%) |
| 816 | Suberin (3%) |
| | Astaxanthin (1%-10%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 817 | Suberin (3%) |
| | Astaxanthin (1%, 2%, 3%, 4%, 5%, 7.5%, or 10%) |
| | Tretinoin (0.025%-1%, 0.025%-0.05%, 0.05%-0.075%, 0.075%-0.1%, |
| | 0.1%-1.0%) |
| 818 | Suberin (3%) |
| | Astaxanthin (1%, 2%, 3%, 4%, 5%, 7.5%, or 10%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 819 | Suberin (3%) |
| | Astaxanthin (1%-10%) |
| | Retinol (0.5%-3%, 0.5%, 1.0%, 1.5%, 2.0%, 2.5%, 3%) |
| 820 | Suberin (3%) |
| | Astaxanthin (1%-10%) |
| | Retinol (0.5%-3%, 0.5%, 1.0%, 1.5%, 2.0%, 2.5%, 3%) |
| | Hydroquinone (2%-4%) |
| 821 | Suberin (3%) |
| | Astaxanthin (1%, 2%, 3%, 4%, 5%, 7.5%, or 10%) |
| | Retinol (0.5%-3%, 0.5%,1.0%, 1.5%, 2.0%, 2.5%, 3%) |
| 822 | Suberin (3%) |
| | Astaxanthin (1%, 2%, 3%, 4%, 5%, 7.5%, or 10%) |
| | Retinol (0.5%-3%, 0.5%, 1.0%, 1.5%, 2.0%, 2.5%, 3%) |
| | Hydroquinone (2%-4%) |
| 823 | Cutin (1%-5%) |
| | Astaxanthin (1%-10%) |
| | Tretinoin (0.025%-1%, 0.025%-0.05%, 0.05%-0.075%, 0.075%-0.1%, |
| | 0.1%-1.0%) |
| 824 | Cutin (1%-5%) |
| | Astaxanthin (1%-10%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 825 | Cutin (1%-5%) |
| | Astaxanthin (1%, 2%, 3%, 4%, 5%, 7.5%, or 10%) |
| | Tretinoin (0.025%-1%, 0.025%-0.05%, 0.05%-0.075%, 0.075%-0.1%, |
| | 0.1%-1.0%) |
| 826 | Cutin (1%-5%) |
| | Astaxanthin (1%, 2%, 3%, 4%, 5%, 7.5%, or 10%) |
| | Tretinoin (0.025%-1%, 0.025%-0.05%, 0.05%-0.075%, 0.075%-0.1%, |
| | 0.1%-1.0%)Hydroquinone (2%-4%) |
| 827 | Cutin (1%-5%) |
| | Astaxanthin (1%, 2%, 3%, 4%, 5%, 7.5%, or 10%) |
| | Retinol (0.5%-3%, 0.5%, 1.0%, 1.5%, 2.0%, 2.5%, 3%) |
| 828 | Cutin (1%-5%) |
| | Astaxanthin (1%, 2%, 3%, 4%, 5%, 7.5%, or 10%) |
| | Retinol (0.5%-3%, 0.5%, 1.0%, 1.5%, 2.0%, 2.5%, 3%) |
| | Hydroquinone (2%-4%) |
| 829 | Arvelexin (10 μM-200 μM) |
| | Tretinoin (0.025%-1%, 0.025%-0.05%, 0.05%-0.075%, 0.075%-0.1%, |
| | 0.1%-1.0%) |
| 830 | Arvelexin (10 μM-200 μM) |
| | Tretinoin (0.025%-1%, 0.025%-0.05%, 0.05%-0.075%, 0.075%-0.1%, |
| | 0.1%-1.0%)Hydroquinone (2%-4%) |
| 831 | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%) |
| 832 | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%)Hydroquinone (2%-4%) |
| 833 | Arvelexin (10 μM-200 μM) |
| | Retinol (0.5%-3%, 0.5%, 1.0%, 1.5%, 2.0%, 2.5%, 3%) |
| 834 | Arvelexin (10 μM-200 μM) |
| | Retinol (0.5%-3%, 0.5%, 1.0%, 1.5%, 2.0%, 2.5%, 3%) |
| | Hydroquinone (2%-4%) |
| 835 | Arvelexin (10 μM, 25 μM, 50 μM, 75 μM, 100 μM, 150 μM, or 200 μM) |
| | Retinol (0.5%-3%, 0.5%, 1.0%, 1.5%, 2.0%, 2.5%, 3%) |
| 836 | Arvelexin (10 μM, 25 μM, 50 μM, 75 μM, 100 μM, 150 μM, or 200 μM) |
| | Retinol (0.5%-3%, 0.5%, 1.0%, 1.5%, 2.0%, 2.5%, 3%) |
| | Hydroquinone (2%-4%) |
| 837 | Suberin (3%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| 838 | Suberin (3%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 839 | Suberin (3%) |
| | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%) |
| 840 | Suberin (3%) |
| | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 841 | Suberin (3%) |
| | Arvelexin (10 μM-200 μM) |
| | Retinol (0.5%-3%, 0.5%, 1.0%, 1.5%, 2.0%, 2.5%, 3%) |
| 842 | Suberin (3%) |
| | Arvelexin (10 μM-200 μM) |
| | Retinol (0.5%-3%, 0.5%, 1.0%, 1.5%, 2.0%, 2.5%, 3%) |
| | Hydroquinone (2%-4%) |
| 843 | Suberin (3%) |
| | Arvelexin (10 μM, 25 μM, 50 μM, 75 μM, 100 μM, 150 μM, or 200 μM) |
| | Retinol (0.5%-3%, 0.5%, 1.0%, 1.5%, 2.0%, 2.5%, 3%) |
| 844 | Suberin (3%) |
| | Arvelexin (10 μM, 25 μM, 50 μM, 75 μM, 100 μM, 150 μM, or 200 μM) |
| | Retinol (0.5%-3%, 0.5%, 1.0%, 1.5%, 2.0%, 2.5%, 3%) |
| | Hydroquinone (2%-4%) |
| 845 | Cutin (1%-5%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| 846 | Cutin (1%-5%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 847 | Cutin (1%-5%) |
| | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%) |
| 848 | Cutin (1%-5%) |
| | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 849 | Cutin (1%-5%) |
| | Arvelexin (10 μM-200 μM) |
| | Retinol (0.5%-3%, 0.5%, 1.0%, 1.5%, 2.0%, 2.5%, 3%) |
| 850 | Cutin (1%-5%) |
| | Arvelexin (10 μM-200 μM) |
| | Retinol (0.5%-3%, 0.5%, 1.0%, 1.5%, 2.0%, 2.5%, 3%) |
| | Hydroquinone (2%-4%) |
| 851 | Cutin (1%-5%) |
| | Arvelexin (10 μM, 25 μM, 50 μM, 75 μM, 100 μM, 150 μM, or 200 μM) |
| | Retinol (0.5%-3%, 0.5%, 1.0%, 1.5%, 2.0%, 2.5%, 3%) |
| 852 | Cutin (1%-5%) |
| | Arvelexin (10 μM, 25 μM, 50 μM, 75 μM, 100 μM, 150 μM, or 200 μM) |
| | Retinol (0.5%-3%, 0.5%, 1.0%, 1.5%, 2.0%, 2.5%, 3%) |
| | Hydroquinone (2%-4%) |
| 853 | Astaxanthin (1%-10%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| 854 | Astaxanthin (1%-10%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 855 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%) |
| 856 | Astaxanthin (1%, 2%, 3%, 4%, 5%, 7.5%, or 10%) |
| | Arvelexin (10 μM, 25 μM, 50 μM, 75 μM, 100 μM, 150 μM, or 200 μM) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 857 | Lignin (1%-30%) |
| | Tretinoin (0.025%-1%, 0.025%-0.05%, 0.05%-0.075%, 0.075%-0.1%, |
| | 0.1%-1.0%) |
| 858 | Lignin (1%-30%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 859 | Lignin (1%-30%) |
| | Retinol (0.5%-3%, 0.5%, 1.0%, 1.5%, 2.0%, 2.5%, 3%) |
| 860 | Lignin (1%-30%) |
| | Retinol (0.5%-3%, 0.5%, 1.0%, 1.5%, 2.0%, 2.5%, 3%) |
| | Hydroquinone (2%-4%) |
| 861 | Lignin (1%-30%) |
| | Astaxanthin (1%-10%) |
| | Tretinoin (0.025%-1%, 0.025%-0.05%, 0.05%-0.075%, 0.075%-0.1%, |
| | 0.1%-1.0%) |
| 862 | Lignin (1%-30%) |
| | Astaxanthin (1%-10%) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 863 | Lignin (1%-30%) |
| | Astaxanthin (1%-10%) |
| | Retinol (0.5%-3%, 0.5%, 1.0%, 1.5%, 2.0%, 2.5%, 3%) |
| 864 | Lignin (1%-30%) |
| | Astaxanthin (1%-10%) |
| | Retinol (0.5%-3%, 0.5%, 1.0%, 1.5%, 2.0%, 2.5%, 3%) |
| | Hydroquinone (2%-4%) |
| 865 | Lignin (1%-30%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| 866 | Lignin (1%-30%) |
| | Arvelexin (10 μM-200 μM) |
| | Tretinoin (0.025%-1%, 0.025%-0.05%, 0.05%-0.075%, 0.075%-0.1%, |
| | 0.1%-1.0%) |
| | Hydroquinone (2%-4%) |
| 867 | Lignin (1%-30%) |
| | Arvelexin (10 μM-200 μM) |
| | Retinol (0.5%-3%, 0.5%, 1.0%, 1.5%, 2.0%, 2.5%, 3%) |
| 868 | Lignin (1%-30%) |
| | Arvelexin (10 μM-200 μM) |
| | Retinol (0.5%-3%, 0.5%, 1.0%, 1.5%, 2.0%, 2.5%, 3%) |
| | Hydroquinone (2%-4%) |

### Example 7: Formulations for Anti-Tumor/Anti-Neoplastic Compositions

Tables 15-18 below include exemplary formulations for topical compositions that include at least one anti-neoplastic agent (e.g., 5-Fluorouracil, Imiquimod, Mechlorethamine HCl) and at least one primary agent (e.g., a plant-derived biopolymer (or extract thereof), arvelexin, astaxanthin). The topical compositions may be applied to a user's body for treating tumors (malignant or benign) and other cancerous or precancerous dermatologic conditions (e.g., basal cell carcinoma, Squamous cell carcinoma, pre-cancerous actinic keratosis, porokeratosis, Bowenoid papulosis). Specific non-limiting examples of indications that may be treated using a retinoid-based topical composition are discussed in additional examples below. Additional active and/or non-active primary or supplemental agents may also be included in or used in conjunction with the formulations below in accordance with the disclosure above. The compositions may be processed and manufactured according to conventional methods known in the art.

The formulations may also include one or more vehicle ingredients to provide a suitable vehicle for topical administration including, but not limited to, those described above. The compositions below may be in the form of a cleanser, toner, moisturizer, scrub, peel, mask, or various emollient, lotion, cream, gel, hydrogel, ointment, solution, foam, wipe, balm, milk, or other suitable vehicle.

Table 15 below includes exemplary formulations for anti-tumor/anti-neoplastic compositions that include 5-Fluorouracil for the treatment of Basal cell carcinoma, Squamous cell carcinoma, pre-cancerous actinic keratosis, porokeratosis, Bowenoid papulosis.

TABLE 15
Anti-tumor compositions that include 5-Fluorouracil. The formulations below
may be used in conjunction with or further include one or more of the additional
supplemental agents disclosed above. Additionally, 5-Fluorouracil (at concentrations
specified herein) may be used alone or in combination with one or more
of the supplemental agents for such conditions.

| Formulation No. | Ingredients (% w/w or w/v, or as indicated) |
|---|---|
| 869 | Suberin (3%), |
| | 5-Fluorouracil (0.01%, 0.05%, 1%, 2%, 2.5%, 5.0%, 7.5%, 10%) |
| 870 | Cutin (1%-5.0) |
| | 5-Fluorouracil (0.01%, 0.05%, 1%, 2%, 2.5%, 5.0%, 7.5%, 10%) |
| 871 | Astaxanthin (1%, 2%, 3%, 4%, 5%, 7.5%, 10%) |
| | 5-Fluorouracil (0.01%, 0.05%, 1%, 2%, 2.5%, 5.0%, 7.5%, 10%) |
| 872 | Suberin (3%) |
| | Astaxanthin (1%, 2%, 3%, 4%, 5%, 7.5%, 10%) |
| | 5-Fluorouracil (0.01%, 0.05%, 1%, 2%, 2.5%, 5.0%, 7.5%, 10%) |
| 873 | Cutin (1%-5%) |
| | Astaxanthin (1%, 2%, 3%, 4%, 5%, 7.5%, 10%) |
| | 5-Fluorouracil (0.01%, 0.05%, 1%, 2%, 2.5%, 5.0%, 7.5%, 10%) |
| 874 | Arvelexin (10 μM, 25 μM, 50 μM, 75 μM, 100 μM, 150 μM, or 200 μM) |
| | 5-Fluorouracil (0.01%, 0.05%, 1%, 2%, 2.5%, 5.0%, 7.5%, 10%) |
| 875 | Suberin (3%) |
| | Arvelexin (10 μM, 25 μM, 50 μM, 75 μM, 100 μM, 150 μM, or 200 μM) |
| | 5-Fluorouracil (0.01%, 0.05%, 1%, 2%, 2.5%, 5.0%, 7.5%, 10%) |
| 876 | Cutin (1%-5%) |
| | Arvelexin (10 μM, 25 μM, 50 μM, 75 μM, 100 μM, 150 μM, or 200 μM) |
| | 5-Fluorouracil (0.01%, 0.05%, 1%, 2%, 2.5%, 5.0%, 7.5%, 10%) |
| 877 | Astaxanthin (1%, 2%, 3%, 4%, 5%, 7.5%, 10%) |
| | Arvelexin (10 μM, 25 μM, 50 μM, 75 μM, 100 μM, 150 μM, or 200 μM) |
| | 5-Fluorouracil (0.01%, 0.05%, 1%, 2%, 2.5%, 5.0%, 7.5%, 10%) |
| 878 | Lignin (1%-30%) |
| | 5-Fluorouracil (0.01%, 0.05%, 1%, 2%, 2.5%, 5.0%, 7.5%, 10%) |
| 879 | Lignin (1%-30%) |
| | Astaxanthin (1%, 2%, 3%, 4%, 5%, 7.5%, 10%) |
| | 5-Fluorouracil (0.01%, 0.05%, 1%, 2%, 2.5%, 5.0%, 7.5%, 10%) |
| 880 | Lignin (1%-30%) |
| | Arvelexin (10 μM, 25 μM, 50 μM, 75 μM, 100 μM, 150 μM, or 200 μM) |
| | 5-Fluorouracil (0.01%, 0.05%, 1%, 2%, 2.5%, 5.0%, 7.5%, 10%) |

Table 16 below includes exemplary formulations for anti-tumor/anti-neoplastic compositions that include imiquimod for the treatment of Basal cell carcinoma, Squamous cell carcinoma, pre-cancerous actinic keratosis, porokeratosis, and Bowenoid papulosis. Imiquimod is also used for treatment of warts (i.e. verruca vulgaris (common warts), genital warts (condyloma acuminatum), plantar warts, periungual warts, flat warts (verruca plana), and Molluscum contagiosum, thus the compositions in Table 16 may also be used to treat such conditions.

TABLE 16
Anti-tumor/anti-neoplastic compositions that include imiquimod. The formulations
below may be used in conjunction with or further include one or more of
the additional supplemental agents disclosed above. Additionally, imiquimod
(at concentrations specified herein) may be used alone or in combination
with one or more of the supplemental agents for such conditions.

| Formulation No. | Ingredients (% w/w or w/v, or as indicated) |
|---|---|
| 881 | Suberin (3%) |
| | Imiquimod (2%, 2.5%, 3.75%, 5%) |
| 882 | Cutin (1%-5.0) |
| | Imiquimod (2%, 2.5%, 3.75%, 5%) |
| 883 | Astaxanthin (1%, 2%, 3%, 4%, 5%, 7.5%, 10%) |
| | Imiquimod (2%, 2.5%, 3.75%, 5%) |
| 884 | Suberin (3%) |
| | Astaxanthin (1%, 2%, 3%, 4%, 5%, 7.5%, 10%) |
| | Imiquimod (2%, 2.5%, 3.75%, 5%) |
| 885 | Cutin (1%-5%) |
| | Astaxanthin (1%, 2%, 3%, 4%, 5%, 7.5%, 10%) |
| | Imiquimod (2%, 2.5%, 3.75%, 5%) |
| 886 | Arvelexin (10 μM, 25 μM, 50 μM, 75 μM, 100 μM, 150 μM, or 200 μM) |
| | Imiquimod (2%, 2.5%, 3.75%, 5%) |
| 887 | Suberin (3%) |
| | Arvelexin (10 μM, 25 μM, 50 μM, 75 μM, 100 μM, 150 μM, or 200 μM) |
| | Imiquimod (2%, 2.5%, 3.75%, 5%) |
| 888 | Cutin (1%-5%) |
| | Arvelexin (10 μM, 25 μM, 50 μM, 75 μM, 100 μM, 150 μM, or 200 μM) |
| | Imiquimod (2%, 2.5%, 3.75%, 5%) |
| 889 | Astaxanthin (1%, 2%, 3%, 4%, 5%, 7.5%, 10%) |
| | Arvelexin (10 μM, 25 μM, 50 μM, 75 μM, 100 μM, 150 μM, or 200 μM) |
| | Imiquimod (2%, 2.5%, 3.75%, 5%) |
| 890 | Lignin (1%-30%) |
| | Imiquimod (2%, 2.5%, 3.75%, 5%) |
| 891 | Lignin (1%-30%) |
| | Astaxanthin (1%, 2%, 3%, 4%, 5%, 7.5%, 10%) |
| | Imiquimod (2%, 2.5%, 3.75%, 5%) |
| 892 | Lignin (1%-30%) |
| | Arvelexin (10 μM, 25 μM, 50 μM, 75 μM, 100 μM, 150 μM, or 200 μM) |
| | Imiquimod (2%, 2.5%, 3.75%, 5%) |

Table 17 below includes exemplary formulations for anti-tumor/anti-neoplastic compositions that include a combination of 5-Fluorouracil and imiquimod for the treatment of Basal cell carcinoma, Squamous cell carcinoma, pre-cancerous actinic keratosis, porokeratosis, and Bowenoid papulosis. The combination is also used for treatment of warts (i.e. verruca vulgaris (common warts), genital warts (condyloma acuminatum), plantar warts, periungual warts, flat warts (verruca plana), and molluscum contagiosum, thus the compositions in Table 17 may also be used to treat such conditions.

TABLE 17
Anti-tumor/anti-neoplastic compositions that include 5-Fluorouracil and imiquimod.
The formulations below may be used in conjunction with or further include
one or more of the additional supplemental agents disclosed above.

| Formulation No. | Ingredients (% w/w or w/v, or as indicated) |
|---|---|
| 893 | Suberin (3%) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 894 | Cutin (1%-5.0) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 895 | Astaxanthin (1%, 2%, 3%, 4%, 5%, 7.5%, 10%) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 896 | Suberin (3%) |
| | Astaxanthin (1%, 2%, 3%, 4%, 5%, 7.5%, 10%) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 897 | Cutin (1%-5%) |
| | Astaxanthin (1%, 2%, 3%, 4%, 5%, 7.5%, 10%) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 898 | Arvelexin (10 μM, 25 μM, 50 μM, 75 μM, 100 μM, 150 μM, or 200 μM) |
| | Imiquimod (2%, 2.5%, 3.75%, 5%) |
| | 5-Fluorouracil (0.01%, 0.05%, 1%, 2%, 2.5%, 5.0%, 7.5%,10%) |
| 899 | Suberin (3%) |
| | Arvelexin (10 μM, 25 μM, 50 μM, 75 μM, 100 μM, 150 μM, or 200 μM) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 900 | Cutin (1%-5%) |
| | Arvelexin (10 μM, 25 μM, 50 μM, 75 μM, 100 μM, 150 μM, or 200 μM) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 901 | Astaxanthin (1%, 2%, 3%, 4%, 5%, 7.5%, 10%) |
| | Arvelexin (10 μM, 25 μM, 50 μM, 75 μM, 100 μM, 150 μM, or 200 μM) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 902 | Lignin (1%-30%) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 903 | Lignin (1%-30%) |
| | Astaxanthin (1%, 2%, 3%, 4%, 5%, 7.5%, 10%) |
| | Imiquimod (2%, 2.5%, 3.75%, 5%) |
| | 5-Fluorouracil (0.01%, 0.05%, 1%, 2%, 2.5%, 5.0%, 7.5%, 10%) |
| 904 | Lignin (1%-30%) |
| | Arvelexin (10 μM, 25 μM, 50 μM, 75 μM, 100 μM, 150 μM, or 200 μM) |
| | Imiquimod (2%, 2.5%, 3.75%, 5%) |
| | 5-Fluorouracil (0.01%, 0.05%, 1%, 2%, 2.5%, 5.0%, 7.5%, 10%) |

Table 18 below includes exemplary formulations for anti-tumor/anti-neoplastic compositions that include Mechorethamine HCl (an alkylating drug also known as VALCHLOR®) for the topical treatment of stage Ia and stage Ib mycosis fungoides-type cutaneous T-cell lymphoma in patients who have received prior skin-directed therapy.

TABLE 18
Anti-tumor/anti-neoplastic compositions that include Mechlorethamine HCl. The
formulations below may be used in conjunction with or further include one or
more of the additional supplemental agents disclosed above. Additionally, Mechlorethamine
(at concentrations specified herein) may be used alone or in combination with
one or more of the supplemental agents for such conditions.

| Formulation No. | Ingredients (% w/w or w/v, or as indicated) |
|---|---|
| 905 | Suberin (3%) |
| | Mechlorethamine HCl (0.01%, 0.02%, 0.03%, 0.04%, 0.05%) |
| 906 | Cutin (1%-5.0) |
| | Mechlorethamine HCl (0.01%, 0.02%, 0.03%, 0.04%, 0.05%) |
| 907 | Astaxanthin (1%, 2%, 3%, 4%, 5%, 7.5%, 10%) |
| | Mechlorethamine HCl (0.01%, 0.02%, 0.03%, 0.04%, 0.05%) |
| 908 | Suberin (3%) |
| | Astaxanthin (1%, 2%, 3%, 4%, 5%, 7.5%, 10%) |
| | Mechlorethamine HCl (0.01%, 0.02%, 0.03%, 0.04%, 0.05%) |
| 909 | Cutin (1%-5%) |
| | Astaxanthin (1%, 2%, 3%, 4%, 5%, 7.5%, 10%) |
| | Mechlorethamine HCl (0.01%, 0.02%, 0.03%, 0.04%, 0.05%) |
| 910 | Arvelexin (10 μM, 25 μM, 50 μM, 75 μM, 100 μM, 150 μM, or 200 μM) |
| | Mechlorethamine HCl (0.01%, 0.02%, 0.03%, 0.04%, 0.05%) |
| 911 | Suberin (3%) |
| | Arvelexin (10 μM, 25 μM, 50 μM, 75 μM, 100 μM, 150 μM, or 200 μM) |
| | Mechlorethamine HCl (0.01%, 0.02%, 0.03%, 0.04%, 0.05%) |
| 912 | Cutin (1%-5%) |
| | Arvelexin (10 μM, 25 μM, 50 μM, 75 μM, 100 μM, 150 μM, or 200 μM) |
| | Mechlorethamine HCl (0.01%, 0.02%, 0.03%, 0.04%, 0.05%) |
| 913 | Astaxanthin (1%, 2%, 3%, 4%, 5%, 7.5%, 10%) |
| | Arvelexin (10 μM, 25 μM, 50 μM, 75 μM, 100 μM, 150 μM, or 200 μM) |
| | Mechlorethamine HCl (0.01%, 0.02%, 0.03%, 0.04%, 0.05%) |
| 914 | Lignin (1%-30%) |
| | Mechlorethamine HCl (0.01%, 0.02%, 0.03%, 0.04%, 0.05%) |
| 915 | Lignin (1%-30%) |
| | Astaxanthin (1%, 2%, 3%, 4%, 5%, 7.5%, 10%) |
| | Mechlorethamine HCl (0.01%, 0.02%, 0.03%, 0.04%, 0.05%) |
| 916 | Lignin (1%-30%) |
| | Arvelexin (10 μM, 25 μM, 50 μM, 75 μM, 100 μM, 150 μM, or 200 μM) |
| | Mechlorethamine HCl (0.01%, 0.02%, 0.03%, 0.04%, 0.05%) |

### Example 8: Formulations for Sunscreen

Tables 19-20 below include exemplary formulations for topical sunscreen compositions that include at least one primary agent (e.g., a plant-derived biopolymer (or extract thereof), arvelexin, astaxanthin). Additional active and/or non-active primary or supplemental agents such as lignin peroxidase, pterostilbene and its derivatives (trans-3,5-dimethoxy-4-hydroxystilbene) (01%-100%, 1 mg-1000 mg, 1.0 μM-500 μM, 1.0 μg/mg-1000 μg/mL), or Equol (4′,7-isoflavandiol) (R-equol, racemic equol or 5S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone) and vitamin D, and in combination with one or more of the anti-inflammatory agents disclosed above, may also be included in or used in conjunction with the formulations below in accordance with the disclosure above. The compositions may be processed and manufactured according to conventional methods known in the art.

The formulations may also include one or more vehicle ingredients to provide a suitable vehicle for topical administration including, but not limited to, those described above. The sunscreen compositions below may be in the form of a spray, lotion, cream, gel, hydrogel, ointment, solution, foam, wipe, or other suitable vehicle.

Table 19 includes exemplary formulations for chemical sunscreen compositions that include one or more of the following chemical sunscreen agents: avobenzone (at a concentration of around 3%), homosalante (at a concentration of around 5%, 10%, 12%, 15%), Octisalante (at a concentration of about 5%), Octocrylene (at a concentration of around 1%, 7%, 2.35%, 6%, 7%), oxybenzone (at a concentration of about 3%, 6%). The formulations below can be in a regular or tinted form

TABLE 19
Chemical sunscreen compositions. The formulations below may
be used in conjunction with or further include one or more
of the additional supplemental agents disclosed above.

| Formulation No. | Ingredients (% w/w or w/v, or as indicated) |
|---|---|
| 917 | Suberin (3%) |
| | Avobenzone (3%) |
| | Homosalante (5%, 10%, 12%, 15%) |
| | Octisalante (5%) |
| | Octocrylene (1.7%, 2.35%, 6%, 7%) |
| 918 | Suberin (3%) |
| | Avobenzone (3%) |
| | Homosalante (5%, 10%, 12%, 15%) |
| | Octisalante (5%) |
| | Oxybenzone (3%, 6%) |
| 919 | Suberin (3%) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |
| 920 | Cutin (1%-5%) |
| | Avobenzone (3%) |
| | Homosalante (5%, 10%, 12%, 15%) |
| | Octisalante (5%), |
| | Octocrylene (1.7%, 2.35%, 6%, 7%) |
| 921 | Cutin (1%-5%) |
| | Avobenzone (3%) |
| | Homosalante (5%, 10%, 12%, 15%) |
| | Octisalante (5%), |
| | Oxybenzone (3%, 6%) |
| 922 | Cutin (1%-5%) |
| | Avobenzone (3%) |
| | Homosalante (5%, 10%, 12%, 15%) |
| | Octisalante (5%), |
| | Octocrylene (1.7%, 2.35%, 6%, 7%) |
| | Oxybenzone (3%, 6%) |
| 923 | Suberin (3%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Octocrylene (1.7%, 2.35%, 6%, 7%) |
| 924 | Suberin (3%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Oxybenzone (3%, 6%) |
| 925 | Suberin (3%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |
| 926 | Cutin (1%-5%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Octocrylene (1.7%, 2.35%, 6%, 7%) |
| 927 | Cutin (1%-5%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Oxybenzone (3%, 6%) |
| 928 | Cutin (1%-5%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |
| 929 | Suberin (3%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Octocrylene (1.7%, 2.35%, 6%, 7%) |
| 930 | Suberin (3%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Oxybenzone (3%, 6%) |
| 931 | Suberin (3%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |
| 932 | Cutin (1%-5%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Octocrylene (1.7%, 2.35%, 6%, 7%) |
| 933 | Cutin (1%-5%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Oxybenzone (3%, 6%) |
| 934 | Cutin (1%-5%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |
| 935 | Lignin (1%-30%) |
| | Avobenzone (3%) |
| | Homosalante (5%, 10%, 12%, 15%) |
| | Octisalante (5%) |
| | Octocrylene (1.7%, 2.35%, 6%, 7%) |
| 936 | Lignin (1%-30%) |
| | Avobenzone (3%) |
| | Homosalante (5%, 10%, 12%, 15%) |
| | Octisalante (5%) |
| | Oxybenzone (3%, 6%) |
| 937 | Lignin (1%-30%) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |
| 938 | Lignin (1%-30%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Octocrylene (1.7%, 2.35%, 6%, 7%) |
| 939 | Lignin (1%-30%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Oxybenzone (3%, 6%) |
| 940 | Lignin (1%-30%) |
| | Astaxanthin (1%, 2%, 3%, 4%, 5%, 7.5%, 10%) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |
| 941 | Lignin (1%-30%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Octocrylene (1.7%, 2.35%, 6%, 7%) |
| 942 | Lignin (1%-30%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Oxybenzone (3%, 6%) |
| 943 | Lignin (1%-30%) |
| | Arvelexin (10 μM, 25 μM, 50 μM, 75 μM, 100 μM, |
| | 150 μM, or 200 μM) |
| | Avobenzone (3%) |
| | Homosalante (5%, 10%, 12%, 15%) |
| | Octisalante (5%) |
| | Octocrylene (1.7%, 2.35%, 6%, 7%) |
| | Oxybenzone (3%, 6%) |

Table 20 includes exemplary formulations for physical sunscreen compositions that include one or more of the following physical sunscreen agents: Titanium Dioxide (at a concentration of around 6%, 9%, 11%), Zinc oxide (at a concentration of around 5%, 7%, 21.6%). The formulations below can be in a regular or tinted form

TABLE 20
Physical sunscreen compositions. The formulations below may
be used in conjunction with or further include one or more
of the additional supplemental agents disclosed above.

| Formulation No. | Ingredients (% w/w or w/v, or as indicated) |
|---|---|
| 944 | Suberin (3%) |
| | Titanium Dioxide (6%, 9%, 11%) |
| | Zinc oxide (5%, 7%, 21.6%) |
| 945 | Cutin (1%-5%) |
| | Titanium Dioxide (6%, 9%, 11%) |
| | Zinc oxide (5%, 7%, 21.6%) |
| 946 | Suberin (3%) |
| | Astaxanthin (1%, 2%, 3%, 4%, 5%, 7.5%, 10%) |
| | Titanium Dioxide (6%, 9%, 11%) |
| | Zinc oxide (5%, 7%, 21.6%) |
| 947 | Cutin (1%-5%) |
| | Astaxanthin (1%, 2%, 3%, 4%, 5%, 7.5%, 10%) |
| | Titanium Dioxide (6%, 9%, 11%) |
| | Zinc oxide (5%, 7%, 21.6%) |
| 948 | Suberin (3%) |
| | Arvelexin (10 μM, 25 μM, 50 μM, 75 μM, 100 μM, |
| | 150 μM, or 200 μM) |
| | Titanium Dioxide (6%, 9%, 11%) |
| | Zinc oxide (5%, 7%, 21.6%) |
| 949 | Cutin (1%-5%) |
| | Arvelexin (10 μM, 25 μM, 50 μM, 75 μM, 100 μM, |
| | 150 μM, or 200 μM) |
| | Titanium Dioxide (6%, 9%, 11%) |
| | Zinc oxide (5%, 7%, 21.6%) |
| 950 | Lignin (1%-30%) |
| | Titanium Dioxide (6%, 9%, 11%) |
| | Zinc oxide (5%, 7%, 21.6%) |
| 951 | Lignin (1%-30%) |
| | Astaxanthin (1%, 2%, 3%, 4%, 5%, 7.5%, 10%) |
| | Titanium Dioxide (6%, 9%, 11%) |
| | Zinc oxide (5%, 7%, 21.6%) |
| 952 | Lignin (1%-30%) |
| | Arvelexin (10 μM, 25 μM, 50 μM, 75 μM, 100 μM, |
| | 150 μM, or 200 μM) |
| | Titanium Dioxide (6%, 9%, 11%) |
| | Zinc oxide (5%, 7%, 21.6%) |

### Example 9: Formulations for Mouthwash/Mouth Rinse

Mouthwash (mouth rinse): used to clean the whole mouth/oral mucosa, prevent cavities, to reduce/control plaque (which may cause gingivitis), restores enamel, bad breath, to refresh breath, tooth decay, and other oral hygiene uses.

There are two kinds of mouthwashes: a) cosmetic mouthwash/mouth rinse, which usually has a flavoring compound (such as peppermint, or spearmint, cinnamon, among other flavors) to temporarily control bad breath/taste but has few or no other benefits such as killing bacteria which are usually associated with bad breath; and b) therapeutic mouthwash/mouth rinse, (available OTC and with prescription)—with and without favoring—which has active ingredients to control/reduce conditions such as tooth decay, plaque, gingivitis, bad breath. The active ingredients may include: Cetylpyridium chloride (used to reduce bad breath), Chlorhexidine (is a prescription germicidal agent in mouthwash/rinse and toothpaste that reduces bacteria in mouth and it is considered to be the most effective antiseptic mouthwash). Other active ingredients include Ovotransferrin to prevent periodontal disease. In addition, essential oils (Such as thymol, menthol, methyl salicylic acid, eucalyptol) are used as natural chemicals which have natural flavor and also have antimicrobial/anti-inflammatory properties used to defend/protect against tooth decay, gingivitis and for general oral infection. This can also be used to clean dentures.

Fluoride (is required by FDA to prevent tooth decay), Peroxide (used as a whitening agent both in mouthwash and toothpaste).

Mouthwashes are often used with various flavors (as discussed above), and in one non-limiting example, the active ingredients of a mouthwash or mouth rinse as discussed herein includes Sodium Fluoride (at a concentration of around 0.02% or 0.01% w/v Fluoride Ion), cetylpyridinium chloride; essential oils; peroxide, Eucalyptol (at a concentration of around 0.092%), Thymol (at a concentration of around 0.064%), Chlorhexidine Gluconate (at a concentration of around 0.12%), Menthol (at a concentration of around 0.042%), Methyl salicylate (at a concentration of around 0.060%).

Additionally, there are a number of inflammatory conditions that affect oral mucosa/gums/tongue (and genital mucosa) such as oral discoid lupus, oral lupus erythematosus, pemphigoid, pemphigoid vulgaris, oral lichen planus, stomatitis, aphthous ulcer, aphthous stomatitis, gingivitis, Bechet's disease as well as infections causing oral mucosa (and genital mucosa) lesions such as oral candidiasis, oral thrush, oral herpes which can be treated by astaxanthin, arvelexin and biopolymers. These products with their multifunctional capabilities (antioxidant, anti-inflammatory, anti-microbial) can be safely used, on a long-term basis, for treatment of these conditions, without having to resort to using long term topical steroids and/or prednisone (i.e. steroid sparing).

Further, most lesions on oral mucosa (also similarly on genital mucosa) tend to be tender to the point that it becomes painful to eat or swallow (which in turn cause weight loss, anorexia, malnutrition which weakens immune system). Therefore, adding topical anesthetic agents (in the form of viscous Lidocaine, Xylocaine, Mepivacaine, Benzocaine, Dibucaine, Proparacaine, Tetracaine, Oxybuprocaine, Pramoxine, Proxymetacaine ranging in concentration from about 0.15%, 0.2%, 0.5%, 1%, 1.5%, 2%, 2.5%, 4.0%, 5.0%), these compositions can be used effectively to reduce the discomfort/tenderness associated with oral (and genital) lesions and allowing patients to eat, swallow, drink, or chew comfortably. Additionally, the antioxidant, anti-inflammatory and antimicrobial effects of astaxanthin, arvelexin and biopolymers as well as some supplemental agents such as pterostilbene and its derivatives (trans-3,5-dimethoxy-4-hydroxystilbene) (01%-100%, 1 mg-1000 mg, 1.0 μM-500 μM, 1.0 μg/mg-1000 μg/mL), or Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone) will accelerate the healing/repair process of the oral (genital) mucosal conditions without having to resort to using topical or systemic steroids as well as Ovotransferrin to prevent periodontal disease,

Tables 21-22 below include exemplary formulations for mouthwash or mouth rinse compositions that include at least one primary agent (e.g., a plant-derived biopolymer (or extract thereof), arvelexin, astaxanthin). Additional active and/or non-active primary or supplemental agents may also be included in or used in conjunction with the formulations below in accordance with the disclosure above. The compositions may be processed and manufactured according to conventional methods known in the art.

The formulations may also include one or more vehicle ingredients to provide a suitable vehicle for topical administration including, but not limited to, those described above. The mouthwash or mouth rinse compositions below may be in the form of a spray, aqueous solution, liquid, lotion, cream, gel, foam, wipe, balm, milk, or other suitable vehicle.

TABLE 21
Mouthwash or mouth rinse compositions. The formulations below
may be used in conjunction with or further include one or
more of the additional supplemental agents disclosed above.

| Formulation No. | Ingredients (% w/w or w/v, or as indicated) |
|---|---|
| 953 | Suberin (3%) |
| | Sodium Fluoride (0.02%) (0.01% w/v Fluoride Ion) |
| 954 | Suberin (3%) |
| | Stannous Fluoride (0.04%-0.05%) |
| 955 | Suberin (3%) |
| | Sodium Fluoride (0.02%, 0,024%, 0.0243%) (0.01% |
| | w/v Fluoride Ion) |
| | Thymol (0.064%) |
| | Menthol (0.042%) |
| | Methyl salicylic acid (0.060%) |
| | Eucalyptol (0.092%) |
| 956 | Suberin (3%) |
| | Stannous Fluoride (0.04%-0.05%) |
| | Thymol (0.064%) |
| | Menthol (0.042%) |
| | Methyl salicylic acid (0.060%) |
| | Eucalyptol (0.092%) |
| 957 | Cutin (1%-5%) |
| | Sodium Fluoride (0.02%) (0.01% w/v Fluoride Ion) |
| 958 | Cutin (1%-5%) |
| | Stannous Fluoride (0.04%-0.05%) |
| 959 | Cutin (1%-5%) |
| | Sodium Fluoride (0.02%) (0.01% w/v Fluoride Ion) |
| | Thymol (0.064%) |
| | Menthol (0.042%) |
| | Methyl salicylic acid (0.060%) |
| 960 | Cutin (1%-5%) |
| | Stannous Fluoride (0.04%-0.05%) |
| | Thymol (0.064%) |
| | Menthol (0.042%) |
| | Methyl salicylic acid (0.060%) |
| 961 | Suberin (3%) |
| | Astaxanthin (1%, 2%, 3%, 4%, 5%, 7.5%, 10%) |
| | Sodium Fluoride (0.02%) (0.01% w/v Fluoride Ion) |
| 962 | Suberin (3%) |
| | Astaxanthin (1%, 2%, 3%, 4%, 5%, 7.5%, 10%) |
| | Stannous Fluoride (0.04%-0.05%) |
| 963 | Suberin (3%) |
| | Astaxanthin (1%, 2%, 3%, 4%, 5%, 7.5%, 10%) |
| | Sodium Fluoride (0.02%) (0.01% w/v Fluoride Ion) |
| | Thymol (0.064%) |
| | Menthol (0.042%) |
| | Methyl salicylic acid (0.060%) |
| 964 | Suberin (3%) |
| | Astaxanthin (1%, 2%, 3%, 4%, 5%, 7.5%, 10%) |
| | Stannous Fluoride (0.04%-0.05%) |
| | Thymol (0.064%) |
| | Menthol (0.042%) |
| | Methyl salicylic acid (0.060%) |
| 965 | Cutin (1%-5%) |
| | Astaxanthin (1%, 2%, 3%, 4%, 5%, 7.5%, 10%) |
| | Sodium Fluoride (0.02%) (0.01% w/v Fluoride Ion) |
| 966 | Cutin (1%-5%) |
| | Astaxanthin (1%, 2%, 3%, 4%, 5%, 7.5%, 10%) |
| | Stannous Fluoride (0.04%-0.05%) |
| 967 | Cutin (1%-5%) |
| | Astaxanthin (1%, 2%, 3%, 4%, 5%, 7.5%, 10%) |
| | Sodium Fluoride (0.02%) (0.01% w/v Fluoride Ion) |
| | Thymol (0.064%) |
| | Menthol (0.042%) |
| | Methyl salicylic acid (0.060%) |
| 968 | Cutin (1%-5%) |
| | Astaxanthin (1%, 2%, 3%, 4%, 5%, 7.5%, 10%) |
| | Stannous Fluoride (0.04%-0.05%) |
| | Thymol (0.064%) |
| | Menthol (0.042%) |
| | Methyl salicylic acid (0.060%) |
| 969 | Suberin (3%) |
| | Arvelexin (10 μM, 25 μM, 50 μM, 75 μM, 100 μM, |
| | 150 μM, or 200 μM) |
| | Sodium Fluoride (0.02%) (0.01% w/v Fluoride Ion) |
| 970 | Suberin (3%) |
| | Arvelexin (10 μM, 25 μM, 50 μM, 75 μM, 100 μM, |
| | 150 μM, or 200 μM) |
| | Stannous Fluoride (0.04%-0.05%) |
| 971 | Cutin (1%-5%) |
| | Arvelexin (10 μM, 25 μM, 50 μM, 75 μM, 100 μM, |
| | 150 μM, or 200 μM) |
| | Sodium Fluoride (0.02%) (0.01% w/v Fluoride Ion) |
| 972 | Cutin (1%-5%) |
| | Arvelexin (10 μM, 25 μM, 50 μM, 75 μM, 100 μM, |
| | 150 μM, or 200 μM) |
| | Stannous Fluoride (0.04%-0.05%) |
| 973 | Cutin (1%-5%) |
| | Arvelexin (10 μM, 25 μM, 50 μM, 75 μM, 100 μM, |
| | 150 μM, or 200 μM) |
| | Thymol (0.064%) |
| | Menthol (0.042%) |
| | Methyl salicylic acid (0.060%) |
| | Sodium Fluoride (0.02%) (0.01% w/v Fluoride Ion) |
| 974 | Cutin (1%-5%) |
| | Arvelexin (10μM, 25μM, 50μM, 75μM, 100μM, |
| | 150μM, or 200μM) |
| | Thymol (0.064%) |
| | Menthol (0.042%) |
| | Methyl salicylic acid (0.060%) |
| | Stannous Fluoride (0.04%-0.05%) |
| 975 | Lignin (1%-30%) |
| | Sodium Fluoride (0.02%) (0.01% w/v Fluoride Ion) |
| 976 | Lignin (1%-30%) |
| | Stannous Fluoride (0.04%-0.05%) |
| 977 | Lignin (1%-30%) |
| | Sodium Fluoride (0.02%, 0,024%, 0.0243%) (0.01% |
| | w/v Fluoride Ion) |
| | Thymol (0.064%) |
| | Menthol (0.042%) |
| | Methyl salicylic acid (0.060%) |
| | Eucalyptol (0.092%) |
| 978 | Lignin (1%-30%) |
| | Stannous Fluoride (0.04%-0.05%) |
| | Thymol (0.064%) |
| | Menthol (0.042%) |
| | Methyl salicylic acid (0.060%) |
| | Eucalyptol (0.092%) |
| 979 | Lignin (1%-30%) |
| | Astaxanthin (1%, 2%, 3%, 4%, 5%, 7.5%, 10%) |
| | Sodium Fluoride (0.02%) (0.01% w/v Fluoride Ion) |
| 980 | Lignin (1%-30%) |
| | Astaxanthin (1%, 2%, 3%, 4%, 5%, 7.5%, 10%) |
| | Stannous Fluoride (0.04%-0.05%) |
| 981 | Lignin (1%-30%) |
| | Astaxanthin (1%, 2%, 3%, 4%, 5%, 7.5%, 10%) |
| | Sodium Fluoride (0.02%) (0.01% w/v Fluoride Ion) |
| | Thymol (0.064%) |
| | Menthol (0.042%) |
| | Methyl salicylic acid (0.060%) |
| 982 | Lignin (1%-30%) |
| | Astaxanthin (1%, 2%, 3%, 4%, 5%, 7.5%, 10%) |
| | Stannous Fluoride (0.04%-0.05%) |
| | Thymol (0.064%) |
| | Menthol (0.042%) |
| | Methyl salicylic acid (0.060%) |
| 983 | Lignin (1%-30%) |
| | Arvelexin (10 μM, 25 μM, 50 μM, 75 μM, 100 μM, |
| | 150 μM, or 200 μM) |
| | Sodium Fluoride (0.02%) (0.01% w/v Fluoride Ion) |
| 984 | Lignin (1%-30%) |
| | Arvelexin (10 μM, 25 μM, 50 μM, 75 μM, 100 μM, |
| | 150 μM, or 200 μM) |
| | Stannous Fluoride (0.04%-0.05%) |
| 985 | Lignin (1%-30%) |
| | Arvelexin (10 μM, 25 μM, 50 μM, 75 μM, 100 μM, |
| | 150 μM, or 200 μM) |
| | Thymol (0.064%) |
| | Menthol (0.042%) |
| | Methyl salicylic acid (0.060%) |
| | Sodium Fluoride (0.02%) (0.01% w/v Fluoride Ion) |
| 986 | Lignin (1%-30%) |
| | Arvelexin (10 μM, 25 μM, 50 μM, 75 μM, 100 μM, |
| | 150 μM, or 200 μM) |
| | Thymol (0.064%) |
| | Menthol (0.042%) |
| | Methyl salicylic acid (0.060%) |
| | Stannous Fluoride (0.04%-0.05%) |

Table 22 includes formulations for mouthwash with local anesthetics to alleviate pain and discomfort associated with conditions that affect oral mucosa, gum, tongue, causing lesions such as: oral discoid lupus, oral lupus erythematosus, pemphigoid, pemphigoid vulgaris, oral lichen planus, stomatitis, gingivitis, aphthous ulcer, aphthous stomatitis, Bechet's disease, as well as infections causing oral mucosa (and genital mucosa) lesions such as oral candidiasis, and oral herpes, oral abrasions, various oral/dental procedures/surgeries, etc.

TABLE 22
Mouthwash or mouth rinse compositions that include a local anesthetic.
The formulations below may be used in conjunction with or further include
one or more of the additional supplemental agents disclosed above.

| Formulation No. | Ingredients (% w/w or w/v, or as indicated) |
|---|---|
| 987 | Suberin (3%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 988 | Suberin (3%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 989 | Cutin (1%-5%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 990 | Cutin (1%-5%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 991 | Cutin (1%-5%) |
| | Astaxanthin (1%, 2%, 3%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) 5 |
| 992 | Cutin (1%-5%) |
| | Astaxanthin (1%, 2%, 3%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 993 | Suberin (3%) |
| | Arvelexin (10 μM, 25 μM, 50 μM, 75 μM, 100 μM, 150 μM, or 200 μM) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 994 | Suberin (3%) |
| | Arvelexin (10 μM, 25 μM, 50 μM, 75 μM, 100 μM, 150 μM, or 200 μM) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 995 | Suberin (3%) |
| | Astaxanthin (1%, 2%, 3%, 4%, 5%, 7.5%, 10%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 996 | Suberin (3%) |
| | Astaxanthin (1%, 2%, 3%, 4%, 5%, 7.5%, 10%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 997 | Cutin (1%-5%) |
| | Arvelexin (10 μM, 25 μM, 50 μM, 75 μM, 100 μM, 150 μM, or 200 μM) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 998 | Cutin (1%-5%) |
| | Arvelexin (10 μM, 25 μM, 50 μM, 75 μM, 100 μM, 150 μM, or 200 μM) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 999 | Lignin (1%-30%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 1000 | Lignin (1%-30%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 1001 | Lignin (1%-30%) |
| | Astaxanthin (1%, 2%, 3%, 4%, 5%, 7.5%, 10%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 1002 | Lignin (1%-30%) |
| | Astaxanthin (1%, 2%, 3%, 4%, 5%, 7.5%, 10%) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |
| 1003 | Lignin (1%-30%) |
| | Arvelexin (10 μM, 25 μM, 50 μM, 75 μM, 100 μM, 150 μM, or 200 μM) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%-5%) |
| 1004 | Lignin (1%-30%) |
| | Arvelexin (10 μM, 25 μM, 50 μM, 75 μM, 100 μM, 150 μM, or 200 μM) |
| | Lidocaine, Xylocaine, Mepivacaine, Benzocaine, Dibucaine, |
| | Proparacaine, Tetracaine, Oxybuprocaine, Pramoxine, or |
| | Proxymetacaine (0.15%, 0.2%, 0.5%, 1%, 1.5%, 2%, 2.5%, |
| | 4.0%, 5.0%) |

### Example 10: Formulations for Toothpaste

Table 23 below include exemplary formulations for toothpaste compositions that include at least one primary agent (e.g., a plant-derived biopolymer (or extract thereof), arvelexin, astaxanthin). Additional active and/or non-active primary or supplemental agents such as pterostilbene and its derivatives (trans-3,5-dimethoxy-4-hydroxystilbene) (01%-100%, 1 mg-1000 mg, 1.0 μM-500 μM, 1.0 μg/mg-1000 μg/mL), or Equol (4′,7-isoflavandiol) (R-equol, racemic equol or S-equol (1%-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone) and other active ingredients. Ovotransferrin to prevent periodontal disease, may also be included in or used in conjunction with the formulations below in accordance with the disclosure above.

Toothpaste used as an anticavity product may include one or more of the following supplemental agents: (i) fluoride as an active ingredient to prevent or treat tooth decay (i.e., “anticaries”); (ii) potassium nitrate and/or Stannous fluoride may be used as desensitizing agents; (iii) pyrophosphates and/or zinc citrate help reduce the build-up of calculus (tartar); (iv) abrasive agents, e.g., modified silica abrasives or enzymes to help clean the teeth and may help whiten teeth by physically removing surface stains (examples of abrasive agents include calcium carbonate, dehydrated silica gels, hydrated aluminum oxides, magnesium carbonate, phosphate salts and silicates); (v) detergents to create foaming action that may help increase the solubility of plaque and accretions during brushing (examples of detergents include sodium lauryl sulfate, sodium N-lauryl sarcosinate); (vi) thickening agents or binders to stabilize the toothpaste formula (examples include mineral colloids, natural gums, seaweed colloids or synthetic cellulose); (vii) flavoring agents and non-caloric sweeteners like saccharin to improve taste (viii) humectants, such as glycerol, propylene glycol, and sorbitol, to help prevent water loss in the toothpaste; (ix) Peroxide, e.g., hydrogen and carbamide peroxides to help reduce intrinsic stains and Ovotransferrin.

A non-limiting example of a toothpaste composition includes sodium fluoride 0.24% (0.04% w/v fluoride ion), triclosan 0.30% (anti-gingivitis), and triclosan (0.03%). Although triclosan in combination with sodium fluoride has FDA approval as a drug which aids in the prevention of caries, plaque, and gingivitis, toothpaste containing triclosan is no longer commercially available as of early in 2019.

The formulations may also include one or more vehicle ingredients to provide a suitable vehicle for topical administration including, but not limited to, those described above. The toothpaste compositions below may be in the form of a paste, spray, aqueous solution, liquid, lotion, cream, ointment, gel, hydrogel, balm, milk, rinse, swash, troches, lozenges, lubricants, foam, wipe, or other suitable vehicle. The compositions may be processed and manufactured according to conventional methods known in the art.

TABLE 23
Toothpaste compositions. The formulations below may be used in conjunction with or
further include one or more of the additional supplemental agents disclosed above.

| Formulation No. | Ingredients (% w/w or w/v, or as indicated) |
|---|---|
| 1005 | Suberin (3%) |
| | Sodium Fluoride (0.02%) (0.01% w/v Fluoride Ion) |
| 1006 | Suberin (3%) |
| | Stannous Fluoride (0.04%-0.05%) |
| 1007 | Cutin (1%-5%) |
| | Sodium Fluoride (0.02%) (0.01% w/v Fluoride Ion) |
| 1008 | Cutin (1%-5%) |
| | Stannous Fluoride (0.04%-0.05%) |
| 1009 | Suberin (3%) |
| | Astaxanthin (1%, 2%, 3%, 4%, 5%, 7.5%, 10%) |
| | Sodium Fluoride (0.02%) (0.01% w/v Fluoride Ion) |
| 1010 | Suberin (3%) |
| | Astaxanthin (1%, 2%, 3%, 4%, 5%, 7.5%, 10%) |
| | Stannous Fluoride (0.04%-0.05%) |
| 1011 | Cutin (1%-5%) |
| | Astaxanthin (1%, 2%, 3%, 4%, 5%, 7.5%, 10%) |
| | Sodium Fluoride (0.02%) (0.01% w/v Fluoride Ion) |
| 1012 | Cutin (1%-5%) |
| | Astaxanthin (1%, 2%, 3%, 4%, 5%, 7.5%, 10%) |
| | Stannous Fluoride (0.04%-0.05%) |
| 1013 | Suberin (3%) |
| | Arvelexin (10 μM, 25 μM, 50 μM, 75 μM, 100 μM, 150 μM, or 200 μM) |
| | Sodium Fluoride (0.02%) (0.01% w/v Fluoride Ion) |
| 1014 | Suberin (3%) |
| | Arvelexin (10 μM, 25 μM, 50 μM, 75 μM, 100 μM, 150 μM, or 200 μM) |
| | Stannous Fluoride (0.04%-0.05%) |
| 1015 | Cutin (1%-5%) |
| | Arvelexin (10 μM, 25 μM, 50 μM, 75 μM, 100 μM, 150 μM, or 200 μM) |
| | Sodium Fluoride (0.02%) (0.01% w/v Fluoride Ion) |
| 1016 | Cutin (1%-5%) |
| | Arvelexin (10 μM, 25 μM, 50 μM, 75 μM, 100 μM, 150 μM, or 200 μM) |
| | Stannous Fluoride (0.04%-0.05%) |
| 1017 | Lignin (1%-30%) |
| | Sodium Fluoride (0.02%) (0.01% w/v Fluoride Ion) |
| 1018 | Lignin (1%-30%) |
| | Stannous Fluoride (0.04%-0.05%) |
| 1019 | Lignin (1%-30%) |
| | Astaxanthin (1%, 2%, 3%, 4%, 5%, 7.5%, 10%) |
| | Sodium Fluoride (0.02%) (0.01% w/v Fluoride Ion) |
| 1020 | Lignin (1%-30%) |
| | Astaxanthin (1%, 2%, 3%, 4%, 5%, 7.5%, 10%) |
| | Stannous Fluoride (0.04%-0.05%) |
| 1021 | Lignin (1%-30%) |
| | Arvelexin (10 μM, 25 μM, 50 μM, 75 μM, 100 μM, 150 μM, or 200 μM) |
| | Sodium Fluoride (0.02%) (0.01% w/v Fluoride Ion) |
| 1022 | Lignin (1%-30%) |
| | Arvelexin (10 μM, 25 μM, 50 μM, 75 μM, 100 μM, 150 μM, or 200 μM) |
| | Stannous Fluoride (0.04%-0.05%) |

### Example 11: Formulations for Shaving Gel or Cream or Foam

Exemplary formulations for topical shaving gel or cream or foam compositions used as a regular shaving gel or cream or foam include plant-derived biopolymer compositions (with or without arvelexin and/or astaxanthin) and with and without supplemental agents such as pterostilbene and its derivatives (trans-3,5-dimethoxy-4-hydroxystilbene) (01%-100%, 1 mg-1000 mg, 1.0 μM-500 μM, 1.0 μg/mg-1000 μg/mL), or Equol (4′,7-isoflavandiol) (R-equol, racemic equol or S-equol (M %-25%, 1 nM-25 nM, 1 mg, 2.5 mg, 5 mg, 7.5 mg, 10 mg, 15 mg, 20 mg, 25 mg, 30 mg, 40 mg, 50 mg-100 mg, or 1 nM-25 nM), or Quercetin (3,3′,4′5,7-Pentahydroxyflavone) including Formulations 1-90 as disclosed and described in the corresponding working examples.

Each of formulations 1-90 may also include one or more anti-microbial agents. In such examples, each of Formulations 1-90 may be combined with each of Formulations 606-715. When used with anti-microbial agents, the composition can be used for as a regular shaving gel/cream/foam, but also to prevent/control and for treatment of folliculitis, hidradenitis suppurativa, MRSA (Methicillin Staphylococcus aureus), impetigo, acne vulgaris, acne rosacea.

### Example 12: Formulations for Shampoos

Exemplary formulations for shampoo compositions include plant-derived biopolymer compositions (with or without arvelexin and/or astaxanthin) including Formulations 1-90 as disclosed and described in the corresponding working examples. Additional active and/or non-active primary or supplemental agents may also be included in or used in conjunction with said formulations in accordance with the disclosure above.

Each of formulations 1-90 may also include one or more anti-microbial agents. In such examples, each of Formulations 1-90 may be combined with each of Formulations 606-715. When used with anti-microbial agents, the composition can be used for as a regular shampoo, but also to prevent, control or treat scalp or beard folliculitis, or acne vulgaris, impetigo, hidradenitis suppurativa, MRSA (Methicillin resistant Staphylococcus aureus), Tinea versicolor, Tinea corporis.

Table 24 below include exemplary formulations for shampoo compositions for treatment of dandruff (seborrheic dermatitis), psoriasis, and treat scalp or beard folliculitis, or acne vulgaris, impetigo, MRSA (Methicillin resistant Staphylococcus aureus), Tinea corporis, Candida intertrigo, Tinea axillaris, Tinea faciei, Tinea versicolor), parasitic infections lice, crab louse or pubic louse. Such compositions may include one or more of the following OTC ingredients: pyrithione zinc 1%, selenium sulfate 1%, ketoconazole 1%, Coal Tar (1%-7%), Salicylic Acid (2%-6%), Oramectin, Eprinomectin, Avermectin, Moxidectin, Abamectin, Ivermectin.

TABLE 24
Active ingredient formulations for over the counter (OTC) topical shampoo
compositions for treatment of dandruff (seborrheic dermatitis), psoriasis,
and tinea versicolor, skin and scalp folliculitis, scalp lice, or body louse.
The formulations below may be used in conjunction with or further include
one or more of the additional supplemental agents disclosed above.

| Formulation No. | Ingredients (% w/w or w/v, or as indicated) |
|---|---|
| 1023 | Suberin (3%) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1024 | Suberin (3%) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1025 | Cutin (1%-5%) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1026 | Suberin (3%), |
| | Astaxanthin (1%, 2%, 3%, 4%, 5%, 7.5%, 10%) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1027 | Cutin (1%-5%) |
| | Astaxanthin (1%, 2%, 3%, 4%, 5%, 7.5%, 10%) and Pyrithione zinc 1%, |
| | or Selenium sulfate 1%, or ketoconazole 1%, or Coal Tar (1%- |
| | 7%), or Salicylic Acid (2%-6%), or miconazole (1.0%, 2.0%, |
| | 3.0%, 4.0%), or clotrimazole (1.0%, 2.0%), or Nystatin (100,000 |
| | units/g; 100000 units/g; 100,000 units; 100,000 units/g with |
| | emollients). |
| 1028 | Suberin (3%) |
| | Arvelexin (10 μM, 25 μM, 50 μM, 75 μM, 100 μM, 150 μM, or 200 μM) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1029 | Cutin (1%-5%) |
| | Arvelexin (10 μM, 25 μM, 50 μM, 75 μM, 100 μM, 150 μM, or 200 μM) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1030 | Lignin (1%-30%) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1031 | Lignin (1%-30%) |
| | Astaxanthin (1%, 2%, 3%, 4%, 5%, 7.5%, 10%) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |
| 1032 | Lignin (1%-30%) |
| | Arvelexin (10 μM, 25 μM, 50 μM, 75 μM, 100 μM, 150 μM, or 200 μM) |
| | Pyrithione zinc 1%, or Selenium sulfate 1%, or ketoconazole 1%, or Coal |
| | Tar (1%-7%), or Salicylic Acid (2%-6%), or miconazole (1.0%, |
| | 2.0%, 3.0%, 4.0%), or clotrimazole (1.0%, 2.0%), or Nystatin |
| | (100,000 units/g; 100000 units/g; 100,000 units; 100,000 units/g |
| | with emollients). |

Table 25 below include exemplary formulations for shampoo compositions for treatment of dandruff (seborrheic dermatitis), psoriasis, and for the treatment of scalp or facial, or skin folliculitis including beard folliculitis, axillary folliculitis, pubic folliculitis, pubic louse, or acne vulgaris, impetigo, MRSA (Methicillin-resistant Staphylococcus aureus), Tinea corporis, Tinea axillaris, Tinea faciei, Candida intertrigo, and Tinea versicolor). Such compositions may include one or more of the following prescribed ingredients: Ketoconazole 2%, ciclopirox 8%, Itraconazole (1%-15%), terbinafine (1%-30%), and Oramectin, Eprinomectin, Avermectin, Moxidectin, Abamectin, Ivermectin.

TABLE 25
Active ingredient formulations for prescription topical shampoo compositions for
treatment of dandruff (seborrheic dermatitis), psoriasis, and for the treatment
of scalp or beard folliculitis, or acne vulgaris, impetigo, MRSA (Methicillin-resistant
Staphylococcus aureus), tinea corporis, tinea axillaris, tinea faciei, and tinea
versicolor. The formulations below may be used in conjunction with or further include
one or more of the additional supplemental agents disclosed above.

| Formulation No. | Ingredients (% w/w or w/v, or as indicated) |
|---|---|
| 1033 | Suberin (3%) |
| | Ketoconazole 2%, or ciclopirox 8%, or Itraconazole (1%-15%), or |
| | Terbinafine (1%-30%) |
| 1034 | Cutin (1%-5%) |
| | Ketoconazole 2%, or ciclopirox 8%, or Itraconazole (1%-15%), or |
| | Terbinafine (1%-30%) |
| 1035 | Suberin (3%) |
| | Astaxanthin (1%, 2%, 3%) 4%, 5%, 7.5%, 10%) |
| | Ketoconazole 2%, or ciclopirox 8%, or Itraconazole (1%-15%), or |
| | Terbinafine1%-30%) |
| 1036 | Cutin (1%-5%) |
| | Astaxanthin (1%, 2%, 3%, 4%, 5%, 7.5%, 10%) |
| | Ketoconazole 2%, or ciclopirox 8%, or Itraconazole (1%-15%), or |
| | Terbinafine (1%-30%) |
| 1037 | Suberin (3%) |
| | Arvelexin (10 μM, 25 μM, 50 μM, 75 μM, 100 μM, 150 μM, or 200 μM) |
| | Ketoconazole 2%, or ciclopirox 8%, or Itraconazole (1%-15%), or |
| | Terbinafine (1%-30%) |
| 1038 | Cutin (1%-5%) |
| | Arvelexin (10 μM, 25 μM, 50 μM, 75 μM, 100 μM, 150 μM, or 200 μM |
| | Ketoconazole 2%, or ciclopirox 8%, or Itraconazole (1%-15%), or |
| | Terbinafine (1%-30%) |
| 1039 | Lignin (1%-30%) |
| | Ketoconazole 2%, or ciclopirox 8%, or Itraconazole (1%-15%), or |
| | terbinafine (1%-30%) |
| 1040 | Lignin (1%-30%) |
| | Astaxanthin (1%, 2%, 3%, 4%, 5%, 7.5%, 10%) |
| | Ketoconazole 2%, or ciclopirox 8%, or Itraconazole (1%-15%), or |
| | Terbinafine (1%-30%) |
| 1041 | Lignin (1%-30%) |
| | Arvelexin (10 μM, 25 μM, 50 μM, 75 μM, 100 μM, 150 μM, or 200 μM) |
| | Ketoconazole 2%, or ciclopirox 8%, or Itraconazole (1%-15%), or |
| | terbinafine (1%-30%) |

### Example 13: Formulations for Deodorants/Antiperspirants

Sweat gland secretion is by itself odorless, and armpit malodor is caused by the microbial biotransformation of the odorless secretion into volatile odorous molecules. Therefore, deodorant products attempt to prevent the growth and activity of the degrading apocrine gland secretion bacteria like Staphylococcus epidermidis and Corynebacterium species. Most currently available deodorant products include antibacterial agents such as quaternary ammonium compounds like triclosan, aluminum salts, and aromatic odor-masking agents. For example, aluminum chlorohydrate (at a concentration of, e.g., 5%, 7%, 10%-20%, 10%-25%) and aluminum zirconium tetrachlorohydrate glycine complex (at a concentration of, e.g., 5%, 7%, 10%, 12%, 15%, 20%), topical glycopyrrolate (0.25%, 0.5%, 0.7%), and Aluminum Chloride (1% 2%, 5%, 10%, 12%, 15%, 17.5%, 20%) are the most frequently used active ingredients in commercial antitranspirants. The FDA has established concentration and dosage formulations of antiperspirant active ingredients for over-the-counter use at 21 CFR § 350.10, including Aluminum chlorohydrate (up to 25%). Aluminum zirconium tetrachlorohydrex gly (up to 20%), and Aluminum zirconium trichlorohydrate (up to 20%).

Although deodorants can be formulated with other, more persistent antimicrobials such as triclosan that slow bacterial growth or with metal chelant compounds such as EDTA, such ingredients are not recommended by the FDA. Triclosan is not considered generally recognized as safe and effective (GRASE) by FDA. Also, the use of aluminum salts in deodorants has been questioned due to possible role in breast cancer and Alzheimer disease. Additionally, in most cases, antiperspirant and deodorants are combined. There are reports of allergies associated with the use of some antiperspirant and deodorants.

Therefore, the combination use of astaxanthin, or arvelexin and biopolymers (suberin, cutin, lignin) provide many advantages over the currently used antiperspirant and deodorant products in many ways. First, they offer natural antibacterial/anti-yeast properties, which will eliminate the need for using triclosan. Also, they prevent and control and treat skin infection of armpits associated with shaving (i.e. folliculitis). Second, they have activity against degrading apocrine gland secretion bacteria like Staphylococcus epidermidis. Therefore, by reducing the population of Staphylococcus epidermidis, they contribute in reducing odor formation. Third, their anti-inflammatory properties, reduces dermatitis (allergic dermatitis) in sensitive individuals which enhances tolerability for using antiperspirant and deodorants. Fourth, because of their extra hydrating capabilities, they tend to keep skin of armpits moist which will be advantageous in reducing friction, especially during exercise, and also act as soothing skin after shaving armpits (after-shave effect) or after laser hair removal procedure. Fifth, these compositions with their antimicrobial/anti-inflammatory/antioxidant properties would enhance wound healing and therefore, will be particularly suitable in patients who have chronic inflammatory skin conditions that affect their armpits such as in patients with history of eczema, atopic dermatitis, psoriasis. Hailey-Hailey disease, pemphigus, candida infection, etc. The anti-perspirant compositions (such as Aluminum Chloride (10%-20%) may also be used for the treatment of axillary (armpit) hyperhidrosis, palmar (hand) and plantar (feet) hyperhidrosis (OTC formulations up to 15%, over 15% by prescription).

Table 26 below includes exemplary formulations for antiperspirant and/or deodorant compositions that include at least one primary agent in addition to a quaternary ammonium compound.

TABLE 26
Active ingredient formulations for antiperspirant and/or deodorant compositions.
The formulations below may be used in conjunction with or further include
one or more of the additional supplemental agents disclosed above.

| Formulation No. | Ingredients (% w/w or w/v, or as indicated) |
|---|---|
| 1042 | Suberin (3%) |
| | Aluminum chlorohydrate (5%-25%), or Aluminum Chloride (10%, |
| | 12%, 15%, 17.5%, 20%), or topical glycopyrrolate (0.25%, |
| | 0.5%, 0.7%) |
| 1043 | Suberin (3%) |
| | Aluminum chlorohydrate (5%, 7%), or Aluminum Chloride (10%, 12%, |
| | 15%, 17.5%, 20%), or topical glycopyrrolate (0.25%, 0.5%, 0.7%) |
| 1044 | Suberin (3%) |
| | Aluminum chlorohydrate (10%-20%), or Aluminum Chloride (10%, |
| | 12%, 15%, 17.5%, 20%), or topical glycopyrrolate (0.25%, |
| | 0.5%, 0.7%) |
| 1045 | Suberin (3%) |
| | Aluminum chlorohydrate (10%-25%), or Aluminum Chloride (10%, |
| | 12%, 15%, 17.5%, 20%), or topical glycopyrrolate (0.25%, |
| | 0.5%, 0.7%) |
| 1046 | Suberin (3%) |
| | Aluminum zirconium tetrachlorohydrate glycine complex (5%-20%), or |
| | Aluminum Chloride (10%, 12%, 15%, 17.5%, 20%), or topical |
| | glycopyrrolate (0.25%, 0.5%, 0.7%) |
| 1047 | Suberin (3%) |
| | Aluminum zirconium tetrachlorohydrate glycine complex (5%, 7%, |
| | 10%, 12%, 15%, 20%), or Aluminum Chloride (10%, 12%, |
| | 15%, 17.5%, 20%), or topical glycopyrrolate (0.25%, 0.5%, 0.7%) |
| 1048 | Cutin (1%-5%) |
| | Aluminum chlorohydrate (5%-25%), or Aluminum Chloride (10%, |
| | 12%, 15%, 17.5%, 20%), or topical glycopyrrolate (0.25%, |
| | 0.5%, 0.7%) |
| 1049 | Cutin (1%-5%) |
| | Aluminum chlorohydrate (5%, 7%, 10%-20%, 10%-25%), or |
| | Aluminum Chloride (10%, 12%, 15%, 17.5%, 20%), or topical |
| | glycopyrrolate (0.25%, 0.5%, 0.7%) |
| 1050 | Cutin (1%, 2%, 3%, 4%, 5%) |
| | Aluminum chlorohydrate (5%, 7%, 10%-20%, 10%-25%), or |
| | Aluminum Chloride (10%, 12%, 15%, 17.5%, 20%), or topical |
| | glycopyrrolate (0.25%, 0.5%, 0.7%) |
| 1051 | Cutin (1%, 2%, 3%, 4%, 5%) |
| | Aluminum chlorohydrate (10%-20%), or Aluminum Chloride (12%, |
| | 15%, 17.5%, 20%), or topical glycopyrrolate (0.25%, 0.5%, 0.7%) |
| 1052 | Cutin (1%, 2%, 3%, 4%, 5%) |
| | Aluminum chlorohydrate (10%-25%), or Aluminum Chloride (10%, |
| | 12%, 15%, 17.5%, 20%), or topical glycopyrrolate (0.25%, |
| | 0.5%, 0.7%) |
| 1053 | Cutin (1%, 2%, 3%, 4%, 5%) |
| | Aluminum chlorohydrate (5%, 7%, 10%-20%, 10%-25%), or |
| | Aluminum Chloride (10%, 12%, 15%, 17.5%, 20%), or topical |
| | glycopyrrolate (0.25%, 0.5%, 0.7%) |
| 1054 | Cutin (1%-5%) |
| | Aluminum chlorohydrate (5%, 7%, 10%-20%, 10%-25%), or |
| | Aluminum Chloride (10%, 12%, 15%, 17.5%, 20%), or topical |
| | glycopyrrolate (0.25%, 0.5%, 0.7%) |
| 1055 | Cutin (1%-5%) |
| | Aluminum zirconium tetrachlorohydrate glycine complex |
| | (5%, 7%. 10%, 12%, 15%, 20%), or Aluminum Chloride (10%, |
| | 12%, 15%, 17.5%, 20%), or topical glycopyrrolate (0.25%, |
| | 0.5%, 0.7%) |
| 1056 | Suberin (3%) |
| | Astaxanthin (1%, 2%, 3%, 4%, 5%, 7.5%, 10%) |
| | Aluminum chlorohydrate (5%, 7%, 10%-20%, 10%-25%) or aluminum |
| | zirconium tetrachlorohydrate glycine complex (5%, 7%. 10%, |
| | 12%, 15%, 20%), or Aluminum Chloride (10%, 12%, 15%, |
| | 17.5%, 20%), or topical glycopyrrolate (0.25%, 0.5%, 0.7%) |
| 1057 | Cutin (1%-5%) |
| | Astaxanthin (1%, 2%, 3%, 4%, 5%, 7.5%, 10%) |
| | Aluminum chlorohydrate (5%, 7%, 10%-20%, 10%-25%) or aluminum |
| | zirconium tetrachlorohydrate glycine complex (5%, 7%. 10%, |
| | 12%, 15%, 20%), or Aluminum Chloride (10%, 12%, 15%, |
| | 17.5%, 20%), or topical glycopyrrolate (0.25%, 0.5%, 0.7%) |
| 1058 | Suberin (3%) |
| | Arvelexin (10 μM, 25 μM, 50 μM, 75 μM, 100 μM, 150 μM, or 200 μM) |
| | Aluminum chlorohydrate (5%, 7%, 10%-20%, 10%-25%) or aluminum |
| | zirconium tetrachlorohydrate glycine complex (5%, 7%. 10%, |
| | 12%, 15%, 20%), or Aluminum Chloride (10%, 12%, 15%, |
| | 17.5%, 20%), or topical glycopyrrolate (0.25%, 0.5%, 0.7%) |
| 1059 | Cutin (1%-5%) and Arvelexin (10 μM, 25 μM, 50 μM, 75 μM, 100 μM, |
| | 150 μM, or 200 μM) |
| | Aluminum chlorohydrate (5%, 7%, 10%-20%, 10%-25%) or aluminum |
| | zirconium tetrachlorohydrate glycine complex |
| | (5%, 7%. 10%, 12%, 15%, 20%), or Aluminum Chloride (10%, |
| | 12%, 15%, 17.5%, 20%), or topical glycopyrrolate (0.25%, |
| | 0.5%, 0.7%) |
| 1060 | Lignin (1%-30%), |
| | Aluminum chlorohydrate (5%, 7%, 10%-20%, 10%-25%) or aluminum |
| | zirconium tetrachlorohydrate glycine complex |
| | (5%, 7%. 10%, 12%, 15%, 20%), or Aluminum Chloride (10%, |
| | 12%, 15%, 17.5%, 20%), or topical glycopyrrolate (0.25%, |
| | 0.5%, 0.7%) |
| 1061 | Lignin (1%-30%) |
| | Astaxanthin (1%, 2%, 3%, 4%, 5%, 7.5%, 10%) |
| | Aluminum chlorohydrate (5%, 7%, 10%-20%, 10%-25%) or aluminum |
| | zirconium tetrachlorohydrate glycine complex (5%, 7%. 10%, |
| | 12%, 15%, 20%), or Aluminum Chloride (10%, 12%, 15%, |
| | 17.5%, 20%), or topical glycopyrrolate (0.25%, 0.5%, 0.7%) |
| 1062 | Lignin (1%-30%) |
| | Arvelexin (10 μM, 25 μM, 50 μM, 75 μM, 100 μM, 150 μM, or 200 μM) |
| | Aluminum chlorohydrate (5%, 7%, 10%-20%, 10%-25%) or aluminum |
| | zirconium tetrachlorohydrate glycine complex (5%, 7%. 10%, |
| | 12%, 15%, 20%), or Aluminum Chloride (10%, 12%, 15%, |
| | 17.5%, 20%), or topical glycopyrrolate (0.25%, 0.5%, 0.7%) |

The formulations in Table 26 (all of which include antiperspirants) may also include one or more deodorants (i.e. Deodorant/antiperspirant) or without deodorants (Antiperspirant only). They may also optionally contain lysozyme (and/or nisin) which may function both as antimicrobial agents as well as preservatives) as well as one or more supplemental agents.

In addition to the ingredients listed in the formulations above, deodorants may contain perfumes, fragrances, or natural essential oils. Essential oils (Such as thymol, menthol, methyl salicylic acid, eucalyptol) intended to mask the odor of perspiration. Deodorant may contain Various natural/synthetic fragrances, Tea tree oil, Geranium, Cypress, peppermint, Rosemary, Clary Sage, Lemon, Bergamot, Eucalyptus, cornstarch/Corn flour, Cyclodextrin, Glucose, Dextrose, Arrowroot powder, Coconut oil, Sandalwood Oil, Shea butter, Beeswax, Grapefruit seed extract, Tapioca Starch, cassava plant extract, Chlorophyll, Green tea extract, Witch-hazel, Sesame seed oil, ethyl cellulose, Lavender, Caprylic/Capric Triglyceride, and Aloe Vera, Ozokerite, Earth wax, Probiotics (i.e. Lactobacillus Acidophilus), baking soda (bicarbonate), Magnesium Hydroxide, alcohol. EDTA (ethylenediaminetetraacetic acid), both for men and women.

### Example 14: Formulations for Treating Hair Loss

Use of minoxidil (Rogaine) has side effects that include inflammatory reaction causing scalp pruritis, scalp sensitivity, dry scalp, flaky scalp irritation or burning sensation at the site of application which at times can be so severe, leading to discontinuation of the use of minoxidil (Rogaine).

Astaxanthin is known to have an inhibitory effect on 5α-reductase, 5α-reductase enzyme converts testosterone to dihydrotestosterone which is associated with androgenic alopecia (scalp hair loss/baldness). The addition of astaxanthin to minoxidil (Rogaine) will proved the following advantages. First, through its anti-inflammatory properties, astaxanthin and/or arvelexin will minimize the side effects of Minoxidil (Rogaine) which will therefore enhance its compliance. In addition, through its inhibitory effects on 5α-reductase enzyme (and hence inhibiting production of dihydrotestosterone), astaxanthin will provide an unexpected and an additive response to the use of Minoxidil (Rogaine) when used alone. The combination of astaxanthin and Minoxidil (Rogaine) will therefore, provide superior result as compared to when Minoxidil is used by itself.

Pityriasis (Malassezia spp) fungus/yeast commonly inhibit scalp, causing inflammation which has been implicated to cause hair loss. Ketoconazole shampoo has anti-fungal properties which inhibits growth of Pityriasis (Malassezia spp), therefore significantly reducing the inflammation of the scalp. Additionally, Ketoconazole shampoo inhibits the 5-alpha reductase enzyme, which converts testosterone to dihydrotestosterone. Therefore, the antifungal properties and its ability to inhibit 5-alpha reductase enzyme make ketoconazole an ideal compound in the treatments of scalp hair loss.

Finasteride inhibits 5-alpha reductase enzyme (type I,) blocking the conversion of testosterone to dihydrotestosterone. Dutasteride (Avodart) is known to block 5-alpha reductase (both type I and Type II). The 5-alpha reductase enzyme inhibitors are known to enhance hair growth. The combination of Finasteride, Dutasteride, Minoxidil, in topical formulations will circumvent the systemic side effects even in the case of androgenic alopecia in females of childbearing age group.

In some embodiments, the dermatological composition used in treatment of various forms of hair loss, alopecia (alopecia arcata, androgenic alopecia, male pattern baldness, alopecia totalis, alopecia universalis) agents described herein include, but are not limited to, astaxanthin. R-equol, racemic equol or S-equol (1%-25%, 1 nM-25 nM, 1 mg-100 mg, 1 mg, 2.5 mg, 5 mg, 7.5 mg, 10 mg, 15 mg, 20 mg, 25 mg, 30 mg, 40 mg, 50 mg-100 mg), ketoconazole 1% shampoo, ketoconazole 2% shampoo, Minoxidil (1%-20%, 1.0%, 2.5%, 5%, 6.0%, 6.5%, 7%, 7.5%, 8.0%, 8.5%, 9.0%, 9.5%, 10%, 15%, 17.5%, 20%). Finasteride (0.001%-10%, 0.001%, 0.005%, 0.1%, 0.25%, 0.5%, 0.75%, 0.8%, 0.9%, 1.0%, 1.5%, 2.0%, 2.5%, 3.0%, 5.0%, 7.5%, 10.%), Dutasteride (0.1%-10%, 0.1%, 0.25%, 0.5%, 0.75%, 0.8%, 0.85%, 0.9%, 0.95%, 1.0%, 1.25%, 1.5%, 1.75%, 2.0%, 2.5%, 5.0%, 7.5%, 10%). Each of the above formulations may be used alone or in combination and with additional one or more of the supplemental agents listed above. In addition, the biopolymers (suberin, cutin, lignin) with their unique physicochemical properties play a superior role in drug delivery across the skin making it ideal for dermal and transdermal drug delivery by increasing the drug permeability. This unique drug delivery capability of biopolymers (suberin, cutin, lignin) together with their anti-inflammatory, antioxidant properties provides an opportunity for a greater efficacy of active drug, quicker onset of action and would allow the use of smaller concentration(s) of the active drug(s) (i.e., minoxidil, finasteride, dutasteride, R-equol, racemic equol or S-equol) and hence reduced side effects.

Table 27 below includes exemplary formulations for topical compositions for treating hair loss both for women and for men. Additional active and/or non-active primary or supplemental agents may also be included in or used in conjunction with the formulations below in accordance with the disclosure above. The compositions may be processed and manufactured according to conventional methods known in the art.

The formulations may also include one or more vehicle ingredients to provide a suitable vehicle for topical administration including, but not limited to, those described above. The compositions below may be in the form of a solution, spray, lotion, cream, gel, hydrogel, foam, wipe, emollient, or other suitable vehicle.

TABLE 27
Compositions for treating hair loss. The formulations below
may be used in conjunction with or further include one or
more of the additional supplemental agents disclosed above.

| Formulation No. | Ingredients (% w/w or w/v, or as indicated) |
|---|---|
| 1063 | Suberin (3%) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, |
| | 7.5%, 10%) |
| 1064 | Cutin (1%-5%) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |
| 1065 | Suberin (3%), |
| | Astaxanthin (1%, 2%, 3%, 4%, 5%, 7.5%, 10%) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |
| 1066 | Cutin (1%-5%) |
| | Astaxanthin (1%, 2%, 3%, 4%, 5%, 7.5%, 10%) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10.%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |
| 1067 | Suberin 3% |
| | Arvelexin (10 μM, 25 μM, 50 μM, 75 μM, 100 μM, 150 μM, or 200 μM) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |
| 1068 | Cutin (1%-5%) |
| | Arvelexin (10 μM, 25 μM, 50 μM, 75 μM, 100 μM, 150 μM, or 200 μM) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |
| 1069 | Lignin (1%-30%), |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |
| 1070 | Lignin (1%-30%), |
| | Astaxanthin (1%, 2%, 3%, 4%, 5%, 7.5%, 10%) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |
| 1071 | Lignin (1%-30%), |
| | Arvelexin (10 μM, 25 μM, 50 μM, 75 μM, 100 μM, 150 μM, or 200 μM) |
| | minoxidil (1.0%, 2.5%, 5%, 6.0%, 6.5%, 7%, 7.5%, 8.0%, 8.5%, 9.0%, |
| | 9.5%, 10%, 15%, 17.5%, 20%) |
| | with or without R-equol, racemic equol or S-equol (1%-25%, 1 nM- |
| | 25 nM, 1 mg, 2.5 mg, 5 mg, 7.5 mg, 10 mg, 15 mg, 20 mg, 25 mg, |
| | 30 mg, 40 mg, 50 mg-100 mg) |
| | with or without ketoconazole shampoo (1%, 2%) |
| | with or without Finasteride (0.001%, 0.005%, 0.1%, 0.25%, 0.5%, |
| | 0.75%, 0.8%, 0.9%, 1.0%, 1.5%, 2.0%, 2.5%, 3.0%, 5.0%, 7.5%, 10%) |
| | with or without Dutasteride (0.1%, 0.25%, 0.5%, 0.75%, 0.8%, 0.85%, |
| | 0.9%, 0.95%, 1.0%, 1.25%, 1.5%, 1.75%, 2.0%, 2.5%, 5.0%, 7.5%, 10%) |

### Example 15: Formulations for Fungal Infections

The unique structure of plant-based biopolymers with their “superhydrophilic” and “superhydrophobic” properties (Koch et al. 2009), will provide several advantages over the current antimicrobial products. The superhydrophobic property of plant-based biopolymers provides protection of nail plate against invading pathogens such as fungi, yeasts, and bacteria, because germination of many micro-organisms such as fungi and reproduction of bacteria are limited by water access (Stosch et al. 2007). Furthermore, the hydrophobic portion of biopolymers, will bind to lipid containing products and biofilims which tend to pile up on the surface of infected nail plate. The hydrophilic properties of biopolymers will provide a cleansing and washing off the microorganisms and their debris. By removing extra-debris and biofilms that tend to build on the surface of the infected nail plate and by allowing extra-cleansing capabilities, the antifungal medications when combined with biopolymers will now have a better ability to penetrate the nail plate and will be able to access and target the invading fungal/yeast organisms easier and provide an added therapeutic efficacy. The antimicrobial, anti-inflammatory and antioxidant properties of biopolymers (and astaxanthin, arvelexin) and additional supplements agents, will therefore provide an unexpected, synergistic response in the treatment of fungal nail infection.

Similarly, in addition to treatment of fungal nail infection, these combination compositions can also be effectively used for treatment of various skin or cutaneous fungal/yeast infections (e.g., Tinea pedis, Tinea cruris, Tinea corporis, Tinea faciei, Tinea axillaris, Tinea capitis, Candida intertrigo, Candida balanitis, Candida vaginitis, and Tinea versicolor).

Table 28 below includes exemplary formulations for topical compositions for treating fungal infection of the nails (onychomycosis) and the skin (Tinea pedis, Tinea cruris, Tinea corporis and Tinea versicolor, Tinea faciei, Tinea axillaris, Tinea capitis, Candida intertrigo, Candida balanitis, Candida vaginitis,) that include at least one primary agent (e.g., a plant-derived biopolymer (or extract thereof), arvelexin, astaxanthin). Additional active and/or non-active primary or supplemental agents may also be included in or used in conjunction with the formulations below in accordance with the disclosure above. The compositions may be processed and manufactured according to conventional methods known in the art.

The formulations may also include one or more vehicle ingredients to provide a suitable vehicle for topical administration including, but not limited to, those described above. The compositions below may be in the form of a spray, lotion, solution, emulsion, cream, gel, hydrogel, ointment, foam, wipe, rinse, shampoo, lubricant, shaving gel, or other suitable vehicle.

TABLE 28
Compositions for treating fungal or yeast infection of the nails or skin.
The formulations below may be used in conjunction with or further include
one or more of the additional supplemental agents disclosed above.

| Formulation No. | Ingredients (% w/w or w/v, or as indicated) |
|---|---|
| 1072 | Suberin (3%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%), or Terbinafine (1%, |
| | 5%, 7.5%, 10%, 12.5%, 15%, 30%) |
| 1073 | Suberin (3%) |
| | Ciclopirox 8%, or Itraconazole (1%, 5%, 7.5%, 10%, 15%), or |
| | Terbinafine (1%, 5%, 7.5%, 10%, 12.5%, 15%, 30%) |
| 1074 | Cutin (1%-5%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%), or Terbinafine (1%, |
| | 5%, 7.5%, 10%, 12.5%, 15%, 30%) |
| 1075 | Cutin (1%-5%) |
| | Ciclopirox 8%, or Itraconazole (1%, 5%, 7.5%, 10%, 15%) or |
| | Terbinafine (1%, 5%, 7.5%, 10%, 12.5%, 15%, 30%) |
| 1076 | Suberin (3%) |
| | Astaxanthin (1%, 2%, 3%, 4%, 5%, 7.5%, 10%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1077 | Suberin (3%) |
| | Astaxanthin (1%, 2%, 3%, 4%, 5%, 7.5%, 10%) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1078 | Cutin (1%-5%) |
| | Astaxanthin (1%, 2%, 3%, 4%, 5%, 7.5%, 10%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1079 | Cutin (1%-5%) |
| | Astaxanthin (1%, 2%, 3%, 4%, 5%, 7.5%, 10%) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1080 | Suberin (3%) |
| | Arvelexin (10 μM, 25 μM, 50 μM, 75 μM, 100 μM, 150 μM, or 200 μM) |
| | Ketoconazole (1.0%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1081 | Suberin (3%) |
| | Arvelexin (10 μM, 25 μM, 50 μM, 75 μM, 100 μM, 150 μM, or 200 μM) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1082 | Cutin (1%-5%) |
| | Arvelexin (10 μM, 25 μM, 50 μM, 75 μM, 100 μM, 150 μM, or 200 μM |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1083 | Cutin (1%-5%) |
| | Arvelexin (10 μM, 25 μM, 50 μM, 75 μM, 100 μM, 150 μM, or 200 μM |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1084 | Lignin (1%-30%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1085 | Lignin (1%-30%) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1086 | Lignin (1%-30%) |
| | Astaxanthin (1%, 2%, 3%, 4%, 5%, 7.5%, 10%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1087 | Lignin (1%-30%) |
| | Astaxanthin (1%, 2%, 3%, 4%, 5%, 7.5%, 10%) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1088 | Lignin (1%-30%) |
| | Arvelexin (10 μM, 25 μM, 50 μM, 75 μM, 100 μM, 150 μM, or 200 μM) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1089 | Lignin (1%-30%) |
| | Arvelexin (10 μM, 25 μM, 50 μM, 75 μM, 100 μM, 150 μM, or 200 μM) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1090 | Lignin (1%-30%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1091 | Lignin (1%-30%) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1092 | Lignin (1%-30%) |
| | Astaxanthin (1%, 2%, 3%, 4%, 7.5%, 10%) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1093 | Lignin (1%-30%) |
| | Astaxanthin (1%, 2%, 3%, 4%, 7.5%, 10%) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |
| 1094 | Lignin (1%-30%) |
| | Arvelexin (10 μM, 25 μM, 50 μM, 75 μM, 100 μM, 150 μM, or 200 μM) |
| | Ketoconazole (1%, 2%), or miconazole (1.0%, 2.0%, 3.0%, 4.0%), or |
| | clotrimazole (1.0%, 2.0%), or Nystatin (100,000 units/g; 100000 |
| | units/g; 100,000 units; 100,000 units/g with emollients), or |
| | Itraconazole (1%, 5%, 7.5%, 10%, 15%) or |
| | Terbinafine (1%, 5%, 7.5%, 10%, 12.5%, 15%, 30%) |
| 1095 | Lignin (1%-30%) |
| | Arvelexin (10 μM, 25 μM, 50 μM, 75 μM, 100 μM, 150 μM, or 200 μM) |
| | Ciclopirox 8% or |
| | Itraconazole (1%, 5%, 7.5%, 10%, 15%) or |
| | Terbinafine (1%, 5%, 7.5%, 10%, 12.5%, 15%, 30%) |

### Example 16: Topical Compositions for Treating Atopic Dermatitis Using Calcineurin Inhibitors

As discussed herein, calcineurin inhibitors such as tacrolimus (0.1% and 0.3%) and pimecrolimus 1% are used to treat atopic dermatitis and are also used by dermatologists for treatment of various inflammatory skin disorders such as psoriasis vulgaris, various forms of dermatitis/eczema, auto-immune skin diseases, and vitiligo both in pediatric and adult populations. However, both tacrolimus and pimecrolimus have side effects such as burning, stinging, itching, peeling and dryness at the skin application site which at times may limit their use. Thus, Table 29 includes compositions that combine tacrolimus and pimecrolimus with plant-derived biopolymers (cutin, suberin, lignin) and/or astaxanthin (or arvelexin) to harness additional anti-inflammatory and antioxidant and UV protection capabilities, as well as potent moisturizing and wound healing properties. Such products are in topical formulations according to the embodiments described herein including, but not limited to, emulsions, lotion, solution, cream, ointment, gel, hydrogel, shampoo, foam, wipe, spray

TABLE 29
Compositions for treating atopic dermatitis, various forms of eczema, dermatitis, psoriasis
vulgaris, and vitiligo using topical “Calcineurin inhibitors: Tacrolimus 0.03%, 0.1%,
or Pimecrolimus 1.0%.” The formulations below may be used in conjunction with or
further include one or more of the additional supplemental agents disclosed above.

| Formulation No. | Ingredients (% w/w or w/v, or as indicated) |
|---|---|
| 1096 | Suberin (3%) |
| | Tacrolimus (0.03%, or 0.1%) or Pimecrolimus 1% |
| 1097 | Cutin (1%-5%) |
| | Tacrolimus (0.03%, or 0.1%) or Pimecrolimus 1.0% |
| 1098 | Suberin (3%) |
| | Astaxanthin (1%, 2%, 3%, 4%, 5%, 7.5%, 10%) |
| | Tacrolimus (0.03%, or 0.1%) or Pimecrolimus 1.0% |
| 1099 | Cutin (1%-5%) |
| | Astaxanthin (1%, 2%, 3%, 4%, 5%, 7.5%, 10%) |
| | Tacrolimus (0.03%, or 0.1%) or Pimecrolimus 1.0% |
| 1100 | Suberin (3%) |
| | Arvelexin (10 μM, 25 μM, 50 μM, 75 μM, 100 μM, 150 μM, or 200 μM) |
| | Tacrolimus (0.03%, or 0.1%) or Pimecrolimus 1.0% |
| 1101 | Cutin (1%-5%) |
| | Arvelexin (10 μM, 25 μM, 50 μM, 75 μM, 100 μM, 150 μM, or 200 μM) |
| | Tacrolimus (0.03%, or 0.1%) or Pimecrolimus 1.0% |
| 1102 | Lignin (1%-30%), |
| | Tacrolimus (0.03%, or 0.1%) or Pimecrolimus 1.0% |
| 1103 | Lignin (1%-30%), |
| | Astaxanthin (1%, 2%, 3%, 4%, 5%, 7.5%, 10%) |
| | Tacrolimus (0.03%, or 0.1%) or Pimecrolimus 1.0% |
| 1104 | Lignin (1%-30%), |
| | Arvelexin (10 μM, 25 μM, 50 μM, 75 μM, 100 μM, 150 μM, or 200 μM) |
| | Tacrolimus (0.03%, or 0.1%) or Pimecrolimus 1.0% |

### Example 17: Topical Compositions for Treating Psoriasis Vulgaris Using Calcipotriene

As discussed herein, topical calcipotriene 0,005% (Dovonex)® is an approved treatment for psoriasis vulgaris, but has side effects such as skin irritation, redness, burning, dryness or peeling of skin, and even worsening of psoriasis, which at times, leads to discontinuation of Calcipotriene. Thus, Table 30 includes compositions that combine calcipotriene with plant-derived biopolymers (cutin, suberin, lignin) and/or astaxanthin (or arvelexin) to harness additional anti-inflammatory and antioxidant and UV protection capabilities, as well as potent moisturizing and wound healing properties. Such products are in topical formulations according to the embodiments described herein including, but not limited to, emulsions, lotion, solution, cream, ointment, gel, hydrogel, shampoo, foam, wipe, spray.

TABLE 30
Compositions for treating psoriasis vulgais using topical calcipotriene 0.005%.
The formulations below may be used in conjunction with or further include
one or more of the additional supplemental agents disclosed above.

| Formulation No. | Ingredients (% w/w or w/v, or as indicated) |
|---|---|
| 1105 | Suberin (3%) |
| | Calcipotriene 0.005% |
| 1106 | Cutin (1%-5%) |
| | Calcipotriene 0.005% |
| 1107 | Suberin (3%) |
| | Astaxanthin (1%, 2%, 3%, 4%, 5%, 7.5%, 10%) |
| | Calcipotriene 0.005% |
| 1108 | Cutin (1%-5%) |
| | Astaxanthin (1%, 2%, 3%, 4%, 5%, 7.5%, 10%) |
| | Calcipotriene 0.005% |
| 1109 | Suberin (3%) |
| | Arvelexin (10 μM, 25 μM, 50 μM, 75 μM, 100 μM, 150 μM, or 200 μM) |
| | Calcipotriene 0.005% |
| 1110 | Cutin (1%-5%) |
| | Arvelexin (10 μM, 25 μM, 50 μM, 75 μM, 100 μM, 150 μM, or 200 μM) |
| | Calcipotriene 0.005% |
| 1111 | Lignin (1%-30%), |
| | Calcipotriene 0.005% |
| 1112 | Lignin (1%-30%), |
| | Astaxanthin (1%, 2%, 3%, 4%, 5%, 7.5%, 10%) |
| | Calcipotriene 0.005% |
| 1113 | Lignin (1%-30%), |
| | Arvelexin (10 μM, 25 μM, 50 μM, 75pM, 100 μM, 150 μM, or 200 μM) |
| | Calcipotriene 0.005% |

### Example 16: Compositions for Dermal Sears and Fibrosis

Cutaneous scars and dermal fibrosis are seen in various conditions including systemic causes such as morphea and scleroderma, lichen sclerosus et atrophicus, linear sclerosis/morphea, and other dermatologic diseases that cause scars (i.e. acne and other inflammatory skin conditions). Scars also form in the process of wound healing secondary to surgery, trauma, accident, burn, ranging in size from small scar to large disfiguring forms as in the case of hypertrophic scars, and keloids. Currently, no effective, tolerable, and safe anti-fibrotic therapy exists. There are multiple pathways involved in the pathogenesis of dermal fibrosis (scarring) including focal adhesion kinase (FAK), Mitogen-activated protein kinase (MEK), and pathways involving tyrosine kinases and multiple other pathways. Nintedanib is an orally available multi-targeted tyrosine kinase inhibitor that inhibits pathways involved in the pathogenesis of dermal fibrosis causing a reduction in scar formation. Defactinib is an orally administered Focal Adhesion Kinase (FAK) inhibitor which is known to attenuate dermal fibrosis and scar formation. Other agents demonstrating antifibrotic, anti-scar properties include Dasatinib, Nilotinib, Verteporfin, Pirfenidone, Silicone, and 5-fluorouracil. The biopolymers (suberin, cutin, lignin) are known to have significant beneficial effects on wound healing and have antioxidant, anticancer, anti-inflammatory, and antifibrotic activities. Furthermore, because of their ability to play a significant role in enhancing dermal/transdermal drug delivery, and increasing skin permeability, the combination of biopolymers (suberin, cutin, lignin) with various topical forms of Nintedanib, Defactinib, Dasatinib or Nilotinib, Verteporfin, Pirfenidone, Silicone, and 5-fluorouracil (either alone or in combination with one or more of the supplemental agents listed above) will provide novel effective and tolerable compositions used in the treatment of dermal fibrotic conditions with additive and synergistic response. This composition will be in the form of lotion, cream, ointment, foam, film, balm, under occlusion, patch, injection, intralesional, pledget, solution (solution used during surgical operation, intraoperative use), irrigation, post-operative use, during cosmetic procedures, and used in conjunction with or in addition to various dressing for wound healing. The concentration of the topical forms of Nintedanib, Defactinib, Dasatinib, Nilotinib, Verteporfin, and Pirfenidone, Silicone, and 5-fluorouracil ranges from 0.00% to 25.00% and the concentration of the biopolymers may be included at any strength indicated above.

### REFERENCES

Anderson M L. A preliminary investigation of the enzymatic inhibition of 5alpha-reduction and growth of prostatic carcinoma cell line LNCap-FGC by natural astaxanthin and Saw Palmetto lipid extract in vitro. J Herb Pharmacother. 2005; 5(1):17-26. PMID: 16093232.
Ascenso A, Ribeiro H, Marques H C, Oliveira H, Santos C, Simões S. Is tretinoin still a key agent for photoaging management? Mini Rev Med Chem. 2014; 14(8):629-41. doi: 10.2174/1389557514666140820102735. PMID: 25141855.
Balci Yuce H, Lektemur Alpan A, Gevrek F, Toker H. Investigation of the effect of astaxanthin on alveolar bone loss in experimental periodontitis. J Periodontal Res. 2018 February; 53(1):131-138. doi: 10.1111/jre.12497. Epub 2017 Oct. 17. PMID: 29044575.
Benohanian A. Antiperspirants and Deodorants. Clin Dermatol. 2001; 19:398-405
Carriço C, Pinto P, Graga A, et al. Design and Characterization of a New Quercus suber-Based Pickering Emulsion for Topical Application. Pharmaceutics. 2019 Mar. 11(3). DOI: 10.3390/pharmaceuticsl 1030131.
Carriço C, Ribeiro H. M., Marto J, Converting cork by-products to ecofriendly cork bioactive ingredients: Novel pharmaceutical and cosmetics applications, Industrial Crops and Products, Volume 125, 2018, Pages 72-84, ISSN 0926-6690, https://doi.org/10.1016/j.indcrop.2018.08.092.
Davinelli et al., Nutrients 2018 10, 522; doi:10.3390-nu10040522
Fernandes Ana, Fernandes Iva, Cruz Luis, Mateus Nuno, Cabral Miguel, and de Freitas Victor, Antioxidant and Biological Properties of Bioactive Phenolic Compounds from Quercus suber L., Journal of Agricultural and Food Chemistry 2009 57 (23), 11154-11160, DOI: 10.1021/jf902093m
Furukawa, F., Kanehara, S., Harano, F. et al. Effects of adenosine 5′-monophosphate on epidermal turnover. Arch Dermatol Res 300, 485-493 (2008). https://doi.org/10.1007/s00403-008-0882-x
Gonçalves Filipa, Correia Patrícia, Silva Susana P., Almeida-Aguiar Cristina, Evaluation of antimicrobial properties of cork, FEMS Microbiology Letters, Volume 363, Issue 3, February 2016, fnv231, https://doi.org/10.1093/femsle/fnv231.
Hwang et al., Int. J. Mol. Sci. 2018, 19(3), 912, doi: 10.3390/ijms19030912.
James A G, Hyliands D, Mycock G. Fatty acid metabolism by cutaneous bacteria and its role in axiliary malodor. World J Microbiol Biotechnol. 2004; 20:787-93
Kishimoto et al., Mar Drugs. 2016 Feb. 5; 14(2):35, doi: 10.3390/md14020035
Koch K, Barthlott W. Superhydrophobic and superhydrophilic plant surfaces: an inspiration for biomimetic materials. Philosophical transactions. Series A, Mathematical, Physical, and Engineering Sciences. 2009 April; 367(1893):1487-1509. DOI: 10.1098/rsta.2009.0022.
Krizková L, Lopes M H, Polónyi J, et al. Antimutagenicity of a suberin extract from Quercus suber cork. Mutation Research. 1999 December; 446(2):225-230. DOI: 10.1016/s1383-5718(99)00190-4.
Leite, Mariana & Lima, Amanda & Kang, Simone & Santos, Maria & Otton, Rosemari. (2014). Effect of astaxanthin and fish oil on enzymatic antioxidant system and ?-amylase activity of salivary glands from rats. Brazilian Journal of Oral Sciences. 13. 58-63. 10.1590/1677-3225v13n1a12.
Meephansan et al., Clin Cosmet Investig Dermatol. 2017 Jul. 13; 10:259-265, doi: 10.2147/CCID.S142795
Miyachi M, Matsuno T, Asano K, Mataga I. Anti-inflammatory effects of astaxanthin in the human gingival keratinocyte line NDUSD-1. J Clin Biochem Nutr. 2015; 56(3):171-178. doi:10.3164/jcbn.14-109
Noferi, M., Masson, E., Merlin, A., Pizzi, A. and Deglise, X. (1997), Antioxidant characteristics of hydrolysable and polyflavonoid tannins: An ESR kinetics study. J. Appl. Polym. Sci., 63: 475-482. doi:10.1002/(SICI)1097-4628(19970124)63:4<475::AID-APP9>3.0.CO;2-O
Paul et al., J. Food Sci. 84(1):19 (2018), doi: 10.1111/1750-3841.14417
Qian, Y., Qiu, X., & Zhu, S. (2015). Lignin: a nature-inspired sun blocker for broad-spectrum sunscreens. Green Chemistry, 17, 320-324.
Santos Sónia A O, Pinto Paula C R O, Silvestre Armando J D, Neto CarlosPascoal. Chemical composition and antioxidant activity of phenolic extracts of cork from Quercus suber L Industrial Crops and products. 2010 May; 31(3):521-526. DOI: 10.1016/j.indcrop.2010.02.001.
Stosch et al. 2007. Efficiency of self-cleaning properties in wheat (Triticum aestivum L.). Appl. Bot. Food Qual. 81, 49-55.
Yoshihisa Y, Rehman M U, Shimizu T. Astaxanthin, a xanthophyll carotenoid, inhibits ultraviolet-induced apoptosis in keratinocytes. Exp Dermatol. 2014 March; 23(3):178-83. doi: 10.1111/exd.12347. PMID: 24521161.

1-28. (canceled)

29. A composition comprising:
a first primary agent comprising a plant-derived biopolymer or extract thereof; and
one or more supplemental agents.

30. The composition of claim 29, wherein the plant-derived biopolymer first primary agent is selected from cutin, suberin, or lignin.

31. The composition of claim 29, further comprising a second primary agent comprising a retinoid.

32. The composition of claim 31, wherein the retinoid is retinol or tretinoin.

33. The composition of claim 31, further comprising a third primary agent comprising astaxanthin or arvelexin.

34. The composition of claim 29, wherein the composition comprises a formulation selected from any one of Formulation Nos. 1 to 1113.

35. The composition of claim 29, further comprising one or more additional agents selected from one or more supplemental agents or one or more additional primary agents.

36. A composition comprising:
a first primary agent comprising a plant-derived biopolymer or extract thereof; and
a second primary agent comprising a retinoid.

37. The composition of claim 36, wherein the plant-derived biopolymer first primary agent is selected from cutin, suberin, or lignin.

38. The composition of claim 36, wherein the retinoid is retinol or tretinoin.

39. The composition of claim 36, further comprising a third primary agent comprising astaxanthin or arvelexin.

40. The composition of claim 36, wherein the composition comprises a formulation selected from any one of Formulation Nos. 1 to 1113.

41. The composition of claim 36, further comprising one or more additional agents selected from one or more supplemental agents or one or more additional primary agents.

42. A dermatological composition for use in treating or preventing a disease or condition comprising:
a first primary agent comprising a plant-derived biopolymer or extract thereof; and
one or more supplemental agents;
wherein dermatological composition is a lotion, a solution, a cream, an ointment, a gel, a hydrogel, a shampoo, a foam, a wipe, a spray, a soap, a mouth wash or mouth rinse, a toothpaste, a deodorant, an antiperspirant, a shaving gel, a shaving cream, a shaving foam, a sunscreen, an anti-microbial, or an exfoliant; and
wherein the disease or condition is selected from an anti-aging condition, acne, acne rosacea, rosacea, retinoid dermatitis, a hyperkeratosis condition, melasma, atopic dermatitis, psoriasis vulgaris, a fungal infection, hair loss, a tumor, or a growth.

43. The composition of claim 42, wherein the plant-derived biopolymer first primary agent is selected from cutin, suberin, or lignin.

44. The composition of claim 42, further comprising a second primary agent comprising a retinoid.

45. The composition of claim 44, wherein the retinoid is retinol or tretinoin.

46. The composition of claim 44, further comprising a third primary agent comprising astaxanthin or arvelexin.

47. The composition of claim 42, wherein the composition comprises a formulation selected from any one of Formulation Nos. 1 to 1113

48. The composition of claim 42, further comprising one or more additional agents selected from one or more supplemental agents or one or more additional primary agents.
